(19) United States
(12) Patent Application Publication
(10) Pub. No.: US 2006/0199252 A1
(43) Pub. Date: Sep. 7, 2006

(54) Genes encoding the synthetic pathway for the production of disorazole

(76) Inventors: Herbert Irschik, Wolfenbuttel (DE); Maren Kopp, Saarbrucken (DE); Rolf Muller, Blieskastel (DE)

Correspondence Address: WILLIAM COLLARD;COLLARD & ROE, P.C., 1077 NORTHERN BOULEVARD, ROSLYN, NY, 11576, US

(21) Appl. No.: 11/330,623

(22) Filed: Jan. 12, 2006

Related U.S. Application Data
(60) Provisional application No. 60/643,899, filed on Jan. 14, 2005.

(30) Foreign Application Priority Data
Jan. 13, 2005 (EP) 05 100 190.7

(51) Int. Cl. C12P 19/62; C12P 7/64; C07H 21/04; C12N 9/10; C12N 1/21

(52) U.S. Cl. 435/76; 435/134; 435/193; 435/252.3; 536/23.2

(57) ABSTRACT

The present invention relates to nucleic acid sequences and proteins derivable therefrom that have been identified in Sorangium cellulosum, which proteins are catalytically active or participate in the biosynthetic pathway of disorazoles. The invention provides novel sequences which are necessary components of the disorazole biosynthetic pathway in addition to genes dszA-D.

The present invention relates to nucleic acid sequences and proteins derivable therefrom which are catalytically active or participate in the biosynthetic pathway of disorazoles. The catalytically active proteins, i.e. enzymes, are also known as polyketide synthases and nonribosomal peptide synthetases.

It is known that myxobacteria produce a large variety of biologically active compounds, also known as secondary metabolites. Among these secondary metabolites, the group of disorazoles has attracted attention as inhibitors for the polymerisation of tubulin, for the induction of apoptosis and for the arrest of the cell cycle or inhibition of cell proliferation at concentrations as low as e.g. 3 pM. The present invention provides nucleic acid sequences and proteins which can be translated from the nucleic acid sequences into catalytically active proteins or proteins participating in the biosynthesis of disorazoles. In cooperation, these translated proteins in vivo and/or in vitro catalyze the formation of disorazoles. Accordingly, the present invention also provides a production process using the nucleic acid sequences and/or proteins derivable therefrom for the production of disorazoles, for example using homologous or heterologous expression of proteins derivable from these nucleic acid sequences in microorganisms for fermentation or the peptides in an immobilized state to produce disorazoles from precursor compounds.

### State of the Art

WO 2004/053065 A2 describes nucleic acid sequences encoding disorazole polyketide synthases DszA, DszB, DszC and DszD obtained from Sorangium cellulosum So ce 12 using transposon generated cosmids. In very general terms, synthetic synthases are described which can be obtained by rearrangement of domains that can be identified in the wildtype disorazole synthase enzymes, namely a ketoreductase domain, a dehydratase domain, an enoylreductase domain, a ketosynthase domain, a nonribosomal protein synthetase domain, a methyltransferase domain, an acyl carrier protein domain, a serine cyclization domain, a serine condensation domain, an adenylation domain, a peptidyl carrier protein domain, a thiolation domain, an oxidase domain, a thioesterase domain, and an acyl transferase domain from a total number of 8 domains in the disorazole synthetase. These domains are predicted from the DNA sequence obtained. However, specific synthetic rearrangements of these domains are not identified. The nucleotide sequence disclosed for the disorazole polyketide synthase and/or nonribosomal peptide synthetase comprises 77294 bp and allegedly includes the coding sequences for DszA, DszB, DszC, DszD and several other open reading frames which are located adjacent one another.

The present invention relates to the group of disorazoles, namely disorazole A1 and derivatives thereof, for example dizorazoles according to the following formulae 1-8 and specific embodiments of these as detailed below: 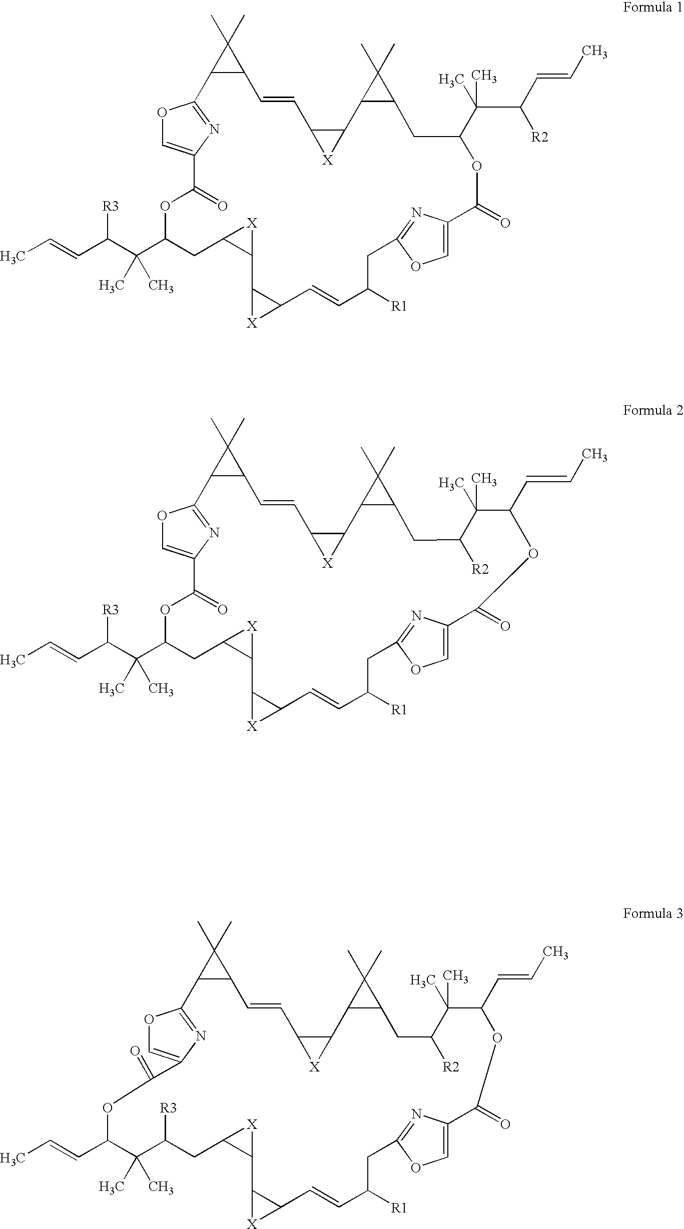 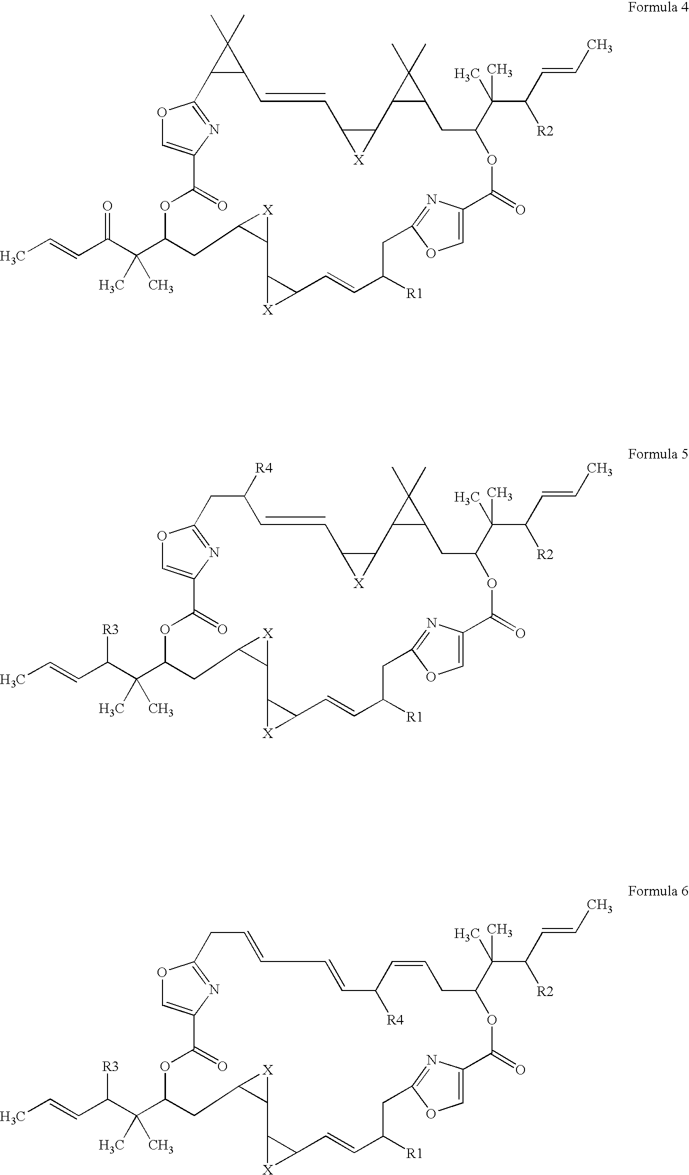 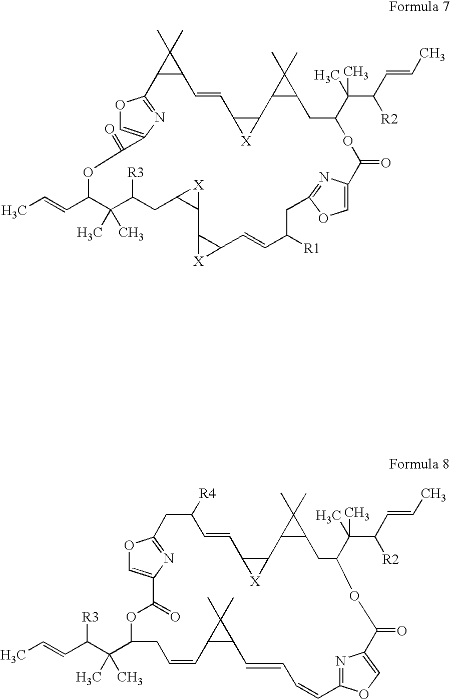
wherein

X represents an O, two vicinal OH, or a single bond and

R1, R2, R3, R4 each represent independently H, OH, OCH₃.

Specific embodiments of general formulae 1-8 are:

Disorazole A1-A7
Disorazole B1-B4
Disorazole C1-C2
Disorazole D1-D5
Disorazole E1-E3
Disorazole F1-F3
Disorazole G1-G3
Disorazole H
Disorazole I

(R. Janssen et al., Liebigs Ann. Chem. 1994, 759-773).

### GENERAL DESCRIPTION OF THE INVENTION

The present invention provides the complete nucleic acid sequences encoding not only a gene cluster but further additional genetic elements which are necessary for correct biosynthesis of disorazoles. The entire biosynthetic gene cluster is disclosed, having high homology to the DszA-D disclosed in WO 2004/053065 A2 including its functional analysis.

The core biosynthetic gene cluster for the biosynthetic pathway for disorazoles comprises genes disA through disD. The gene disA is preceded by a putative ribosomal binding site located 11 base pairs upstream from the designated start codon (GTG). DisB presumably starts with an ATG and a putative ribosomal binding site could be localized 7 base pairs upstream from the start codon. Arranged with disA and disB, which are polyketide synthases, in one transcriptional unit is disC, the latter encoding a mixed polyketide synthase/nonribosomal peptide synthetase. DisC most likely starts with an ATG, preceded by a putative ribosomal binding site located 8 base pairs upstream. An alternative start codon of disC could be found 36 base pairs downstream of the putative start codon. Downstream this transcriptional unit of disA, disB and disC, a probable transcription terminator is located.

Following orf 9, located downstream of the transcriptional unit disA through disC, disD was identified having its putative ribosomal binding site 7 base pairs upstream its start codon. The gene disD shows significant similarities to the bifunctional proteins LnmG from the leinamycin biosynthetic gene cluster and to MmpIII from the mupirocin biosynthetic gene cluster. The C-terminus of DisD has close sequence similarity to the oxidoreductase superfamily. From a total of four transposon mutants, listed in Table 3 below, plasmids were recovered, harbouring the hygromycin resistance gene and the λpir dependent origin of replication (ori) R6K together with parts of chromosomal DNA of Sorangium cellulosum So ce 12 which originally flanked the transposition site. A computer assisted analysis of the chromosomal DNA portions using BLAST searches identified two of the proteins predicted from the recovered DNA portions as putative fragments of a polyketide synthase and a nonribosomal peptide synthetase. Using these two chromosomal DNA portions as probes for hybridization with a BAC library, previously established for Sorangium cellulosum So ce 12, sequencing of hybridizing BAC clones yielded orfs encoding proteins participating in the biosynthesis of disorazoles, which are summarised in Table 1 below.

### DETAILED DESCRIPTION OF THE INVENTION

When analysing the biosynthetic pathway for the production of disorazoles, the genomic DNA of Sorangium cellulosum So ce 12 has been analyzed to identify the genes whose translation products are necessary components of the synthetic pathway, finally producing disorazoles including known variants or derivatives of disorazole A, e. g. according to formulae 1-8 above. The gene cluster encoding the enzymes catalyzing the biosynthesis of disorazoles comprises the translation products of disA, disB, disC, disD. It is possible that translation products from open reading frame (orf) orf 9, arranged between disC and disD, may participate in or be beneficial to the biosynthesis of disorazoles.

In the following, reference is made to the figures, wherein

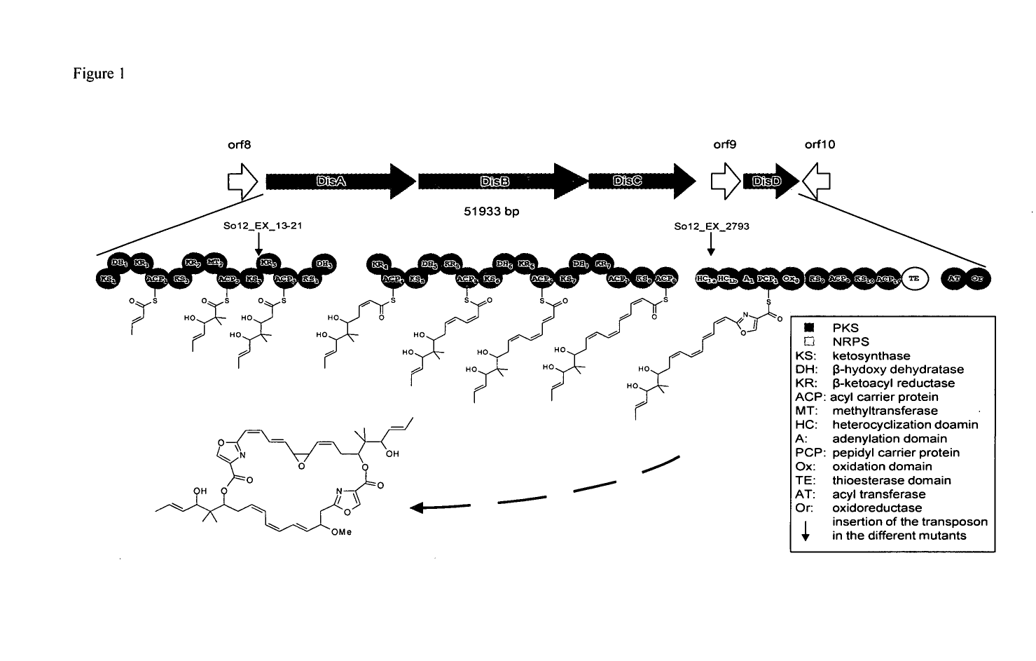

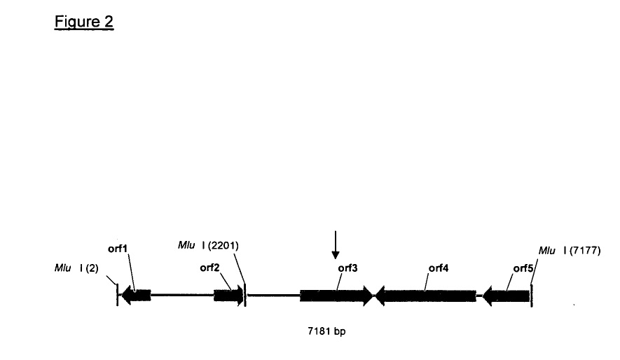

FIG. 1 is a schematic representation of the synthetic pathway for disorazoles,

FIG. 2 schematically shows the arrangement of genes adjacent to the insertion site of the transposon in the transposon mutant So ce 12_EXI_IE-2 and sequenced from its plasmid pTn-Rec_IE-2, and

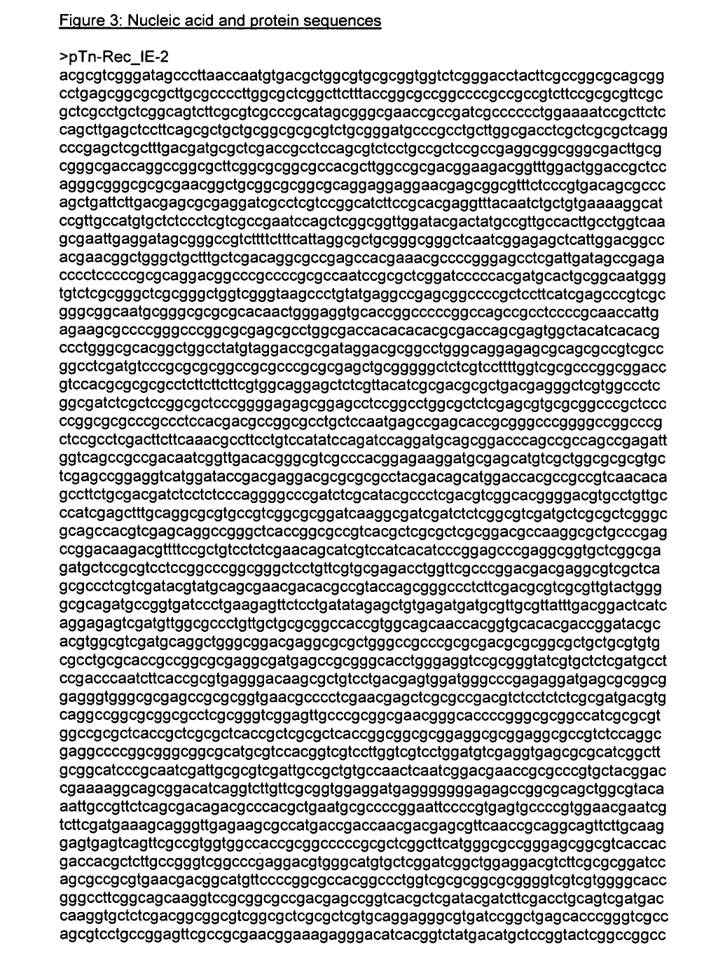

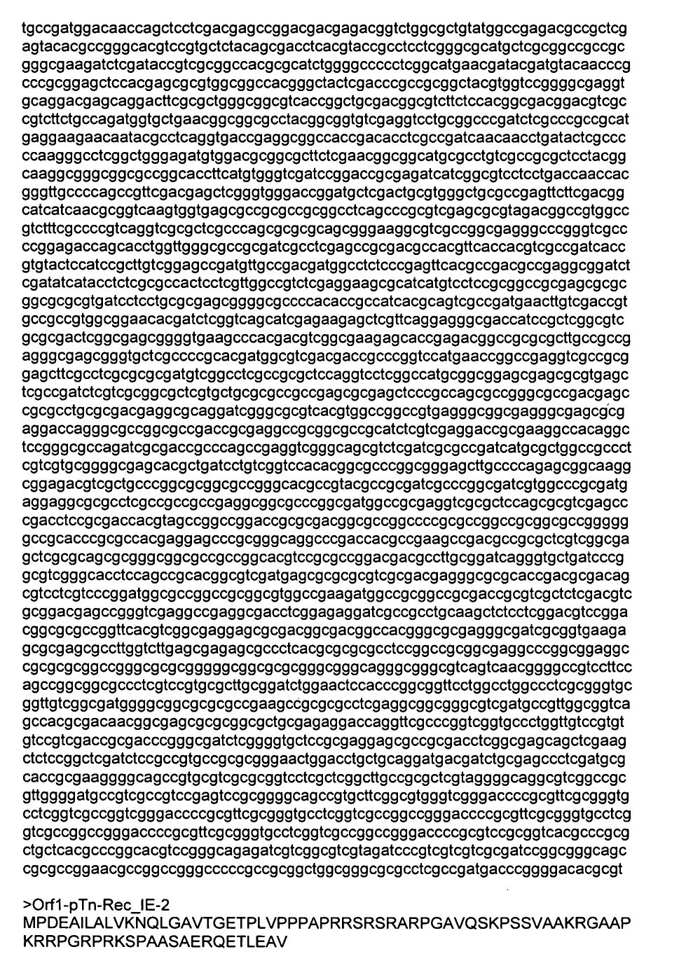

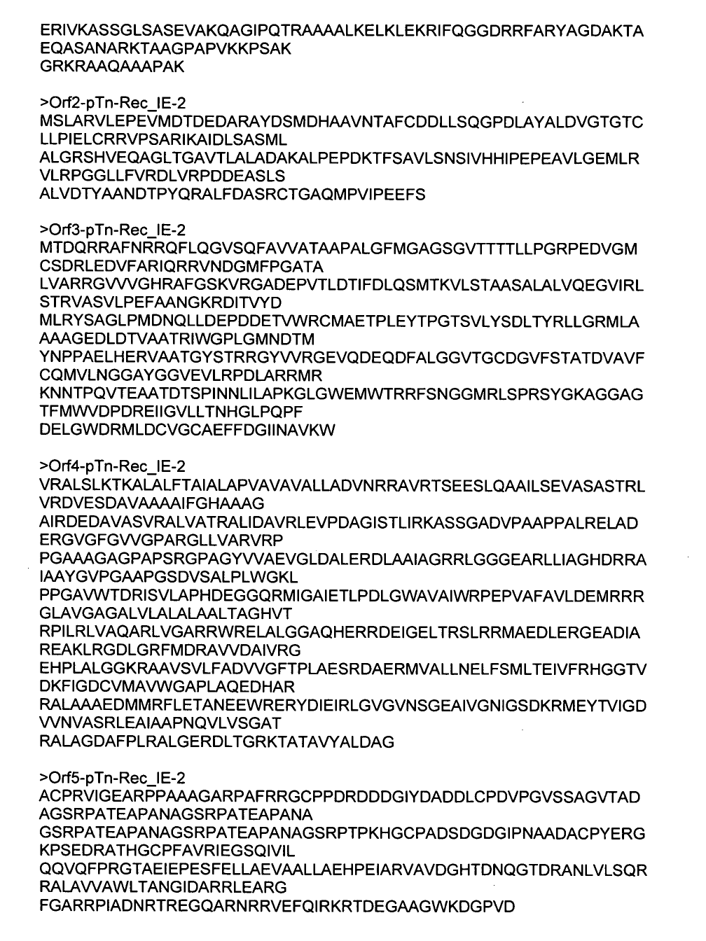

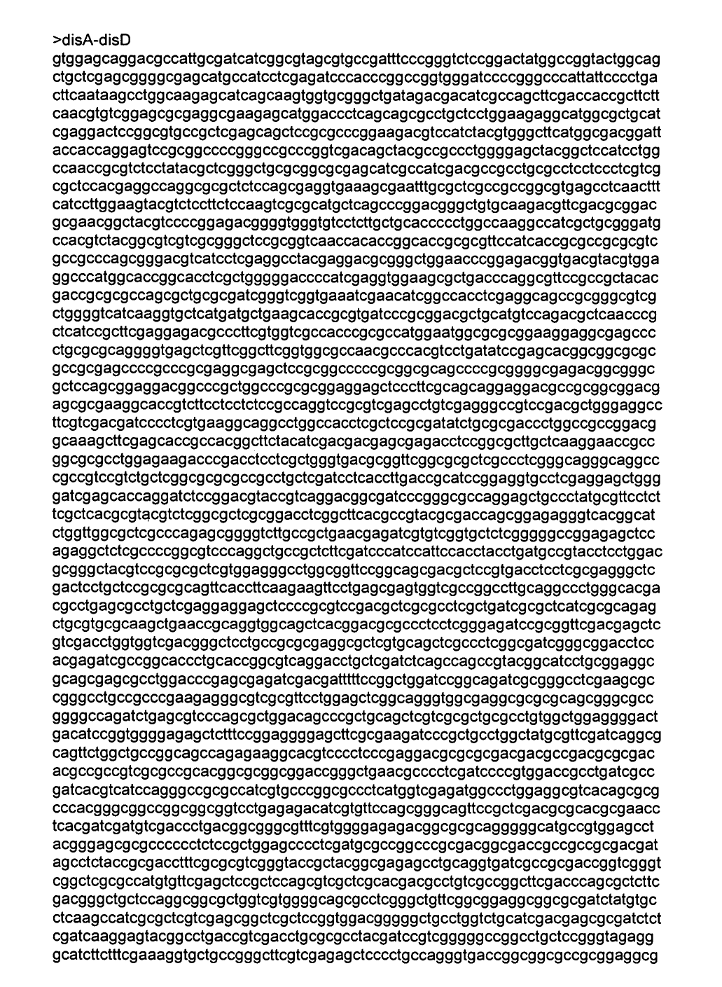

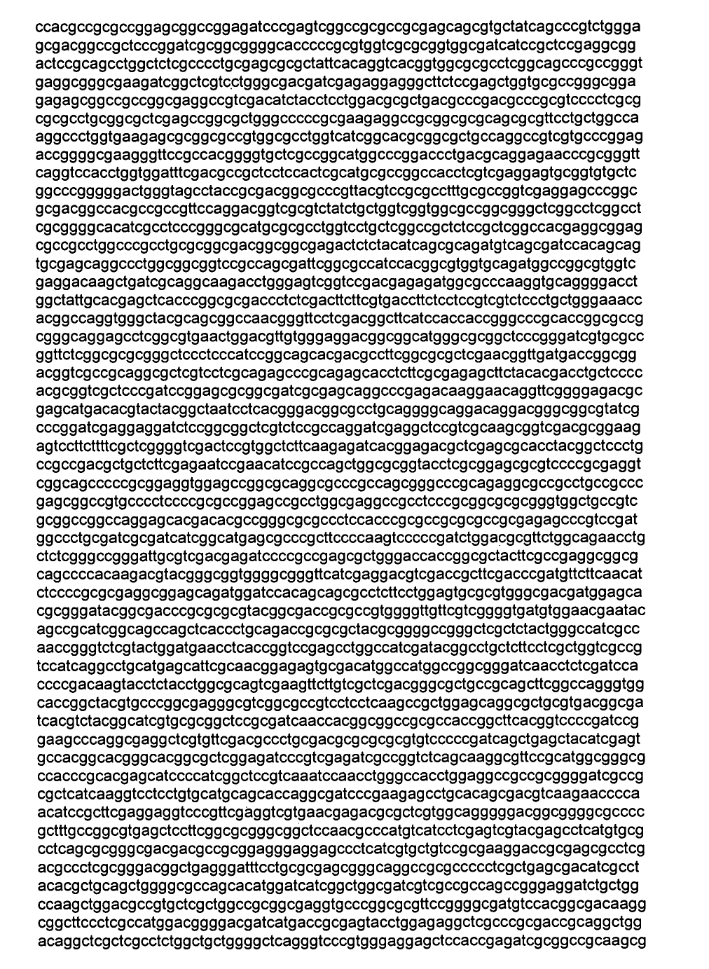

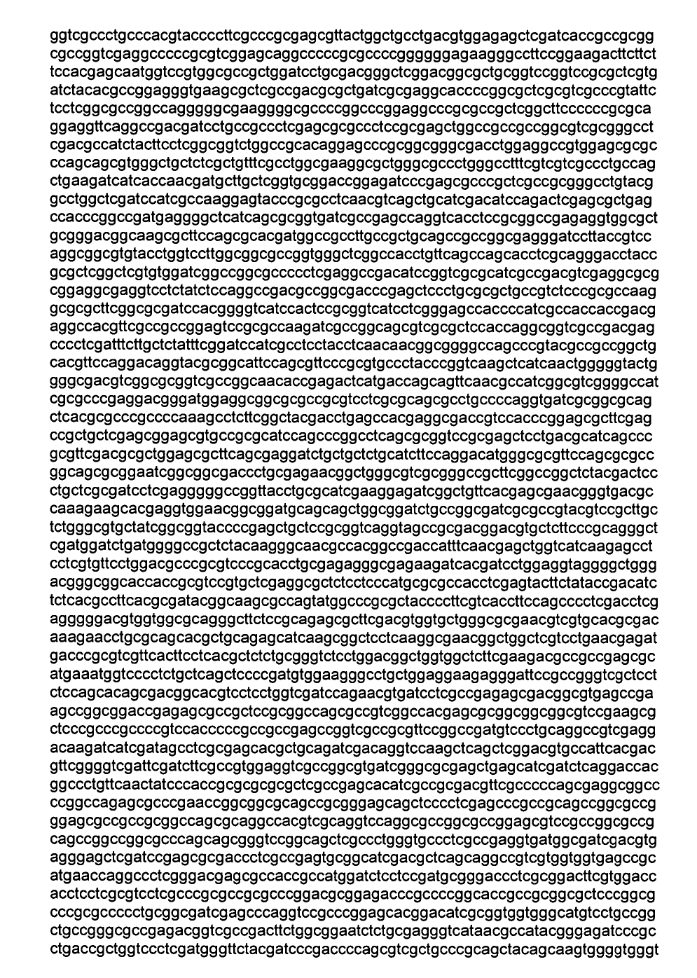

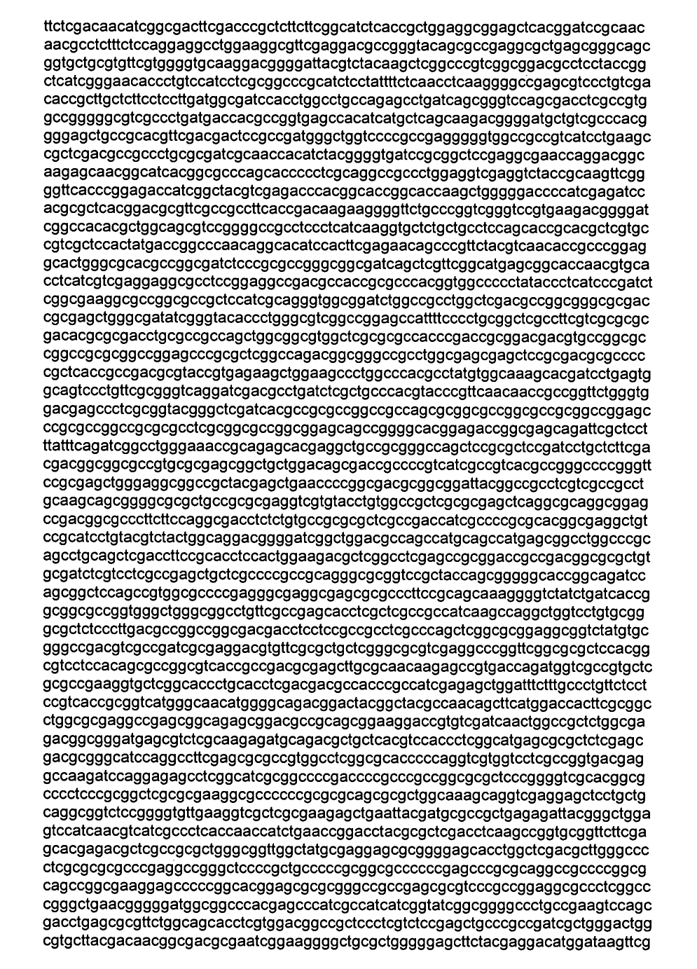

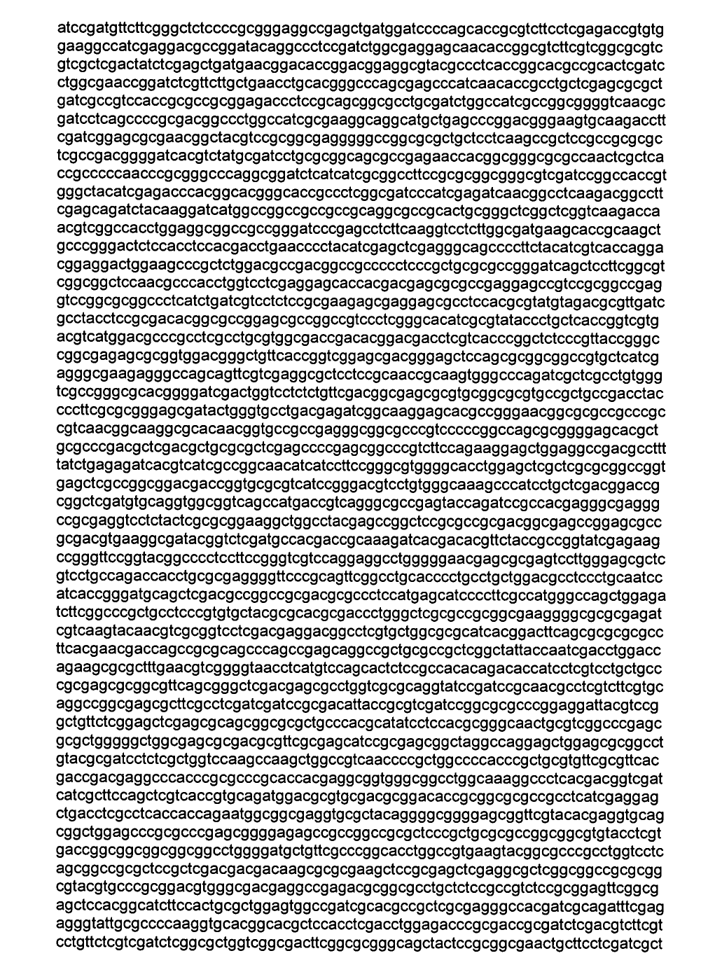

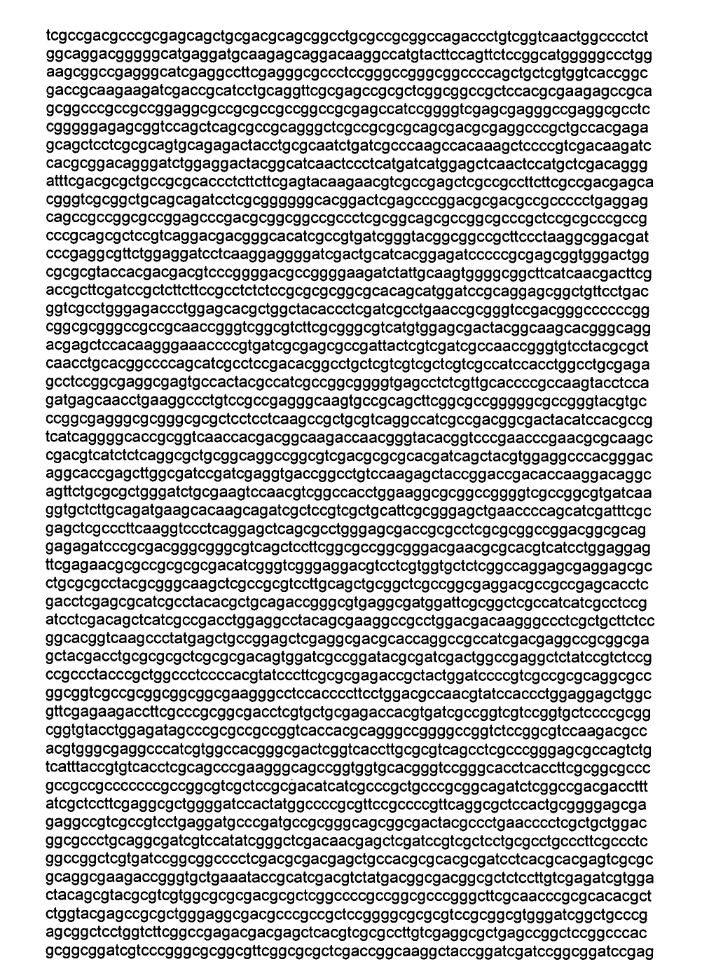

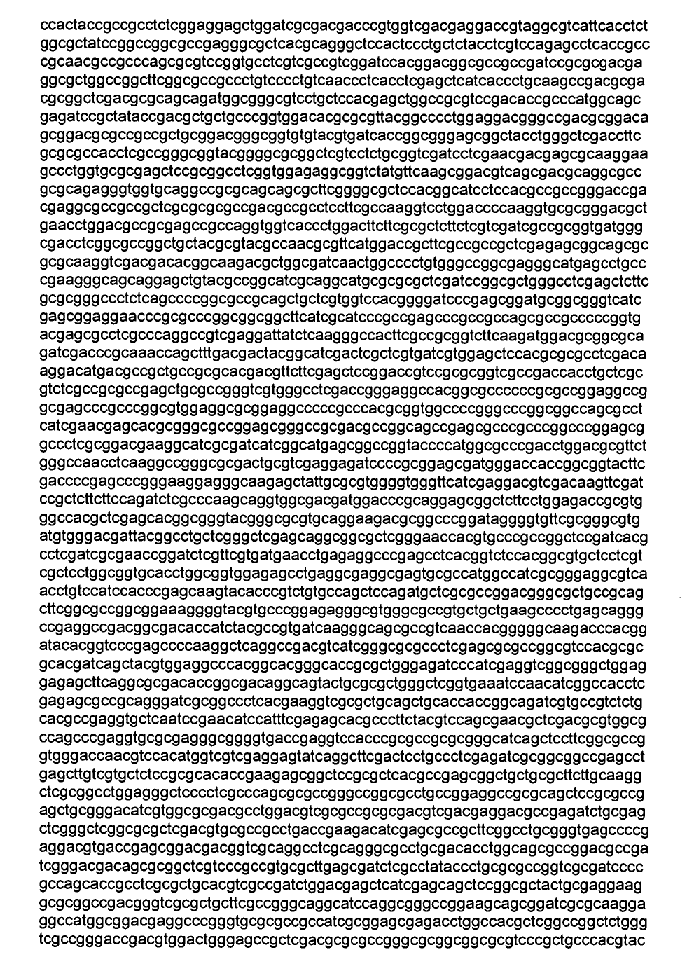

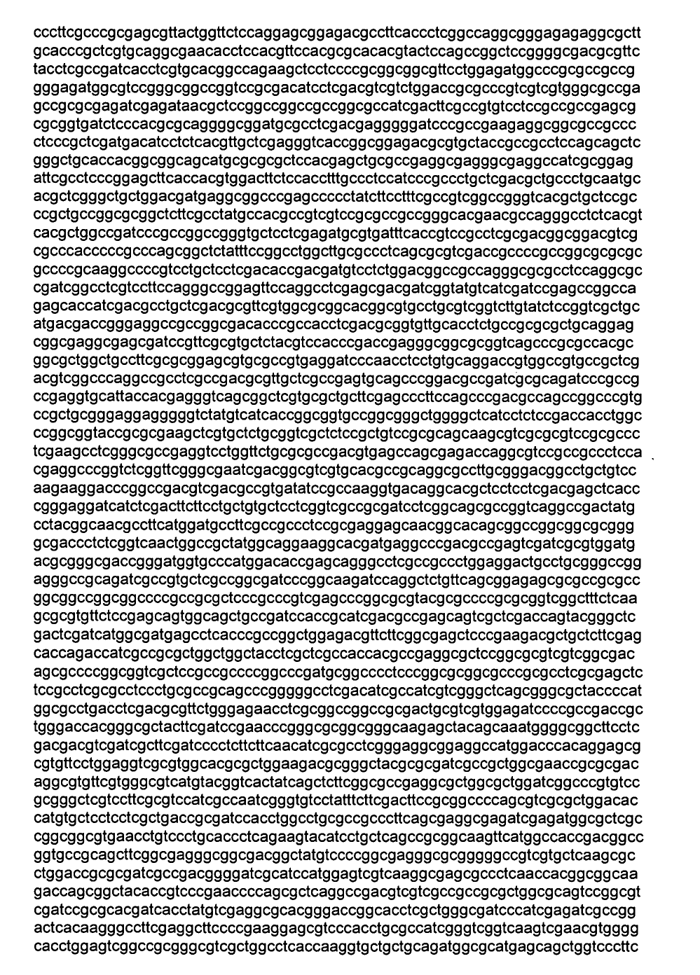

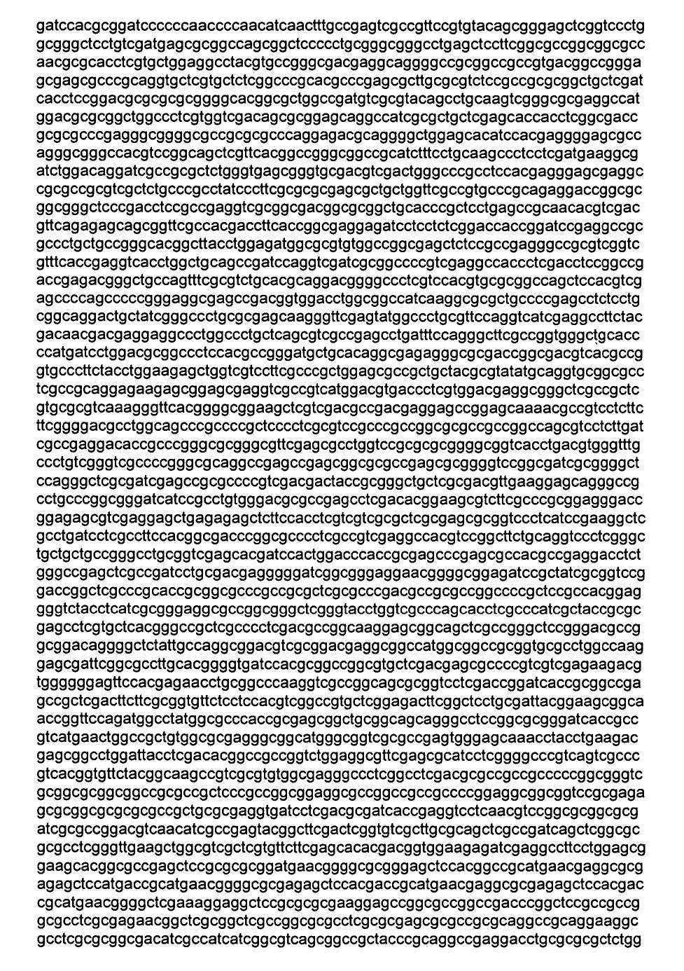

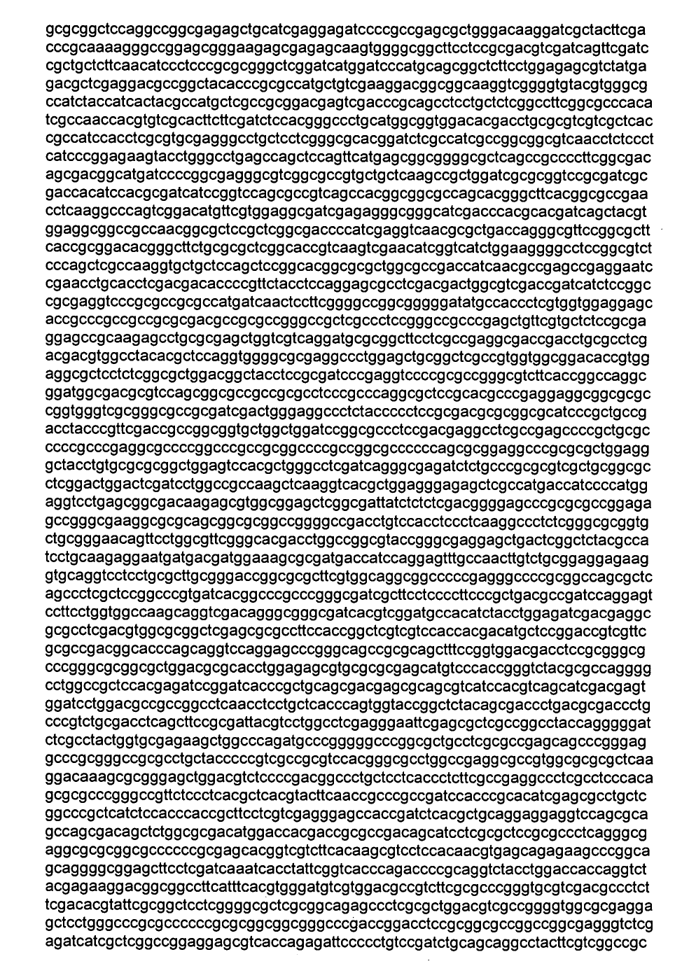

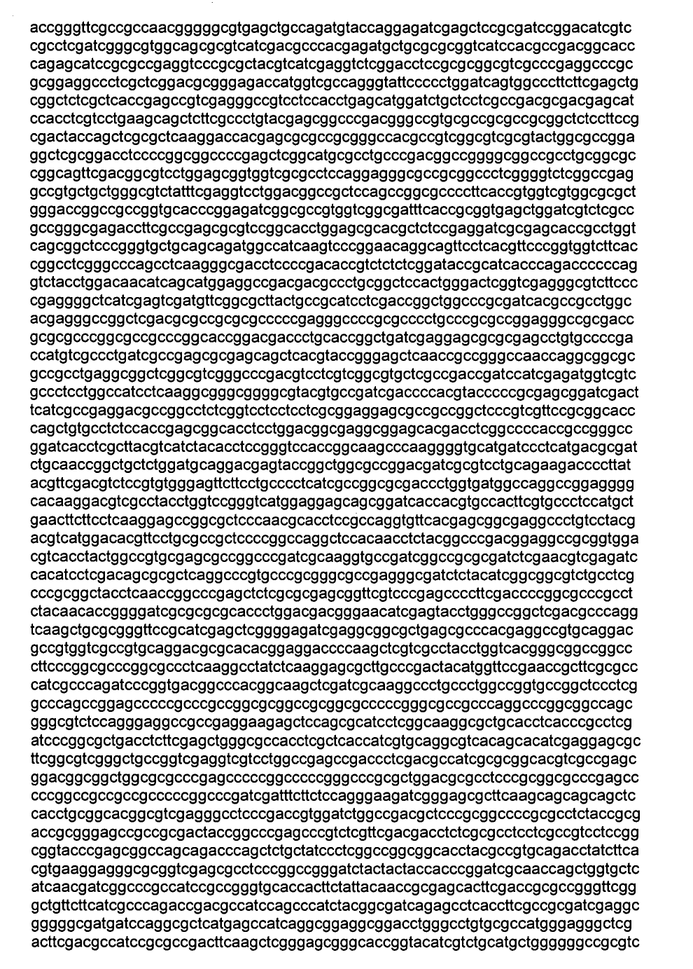

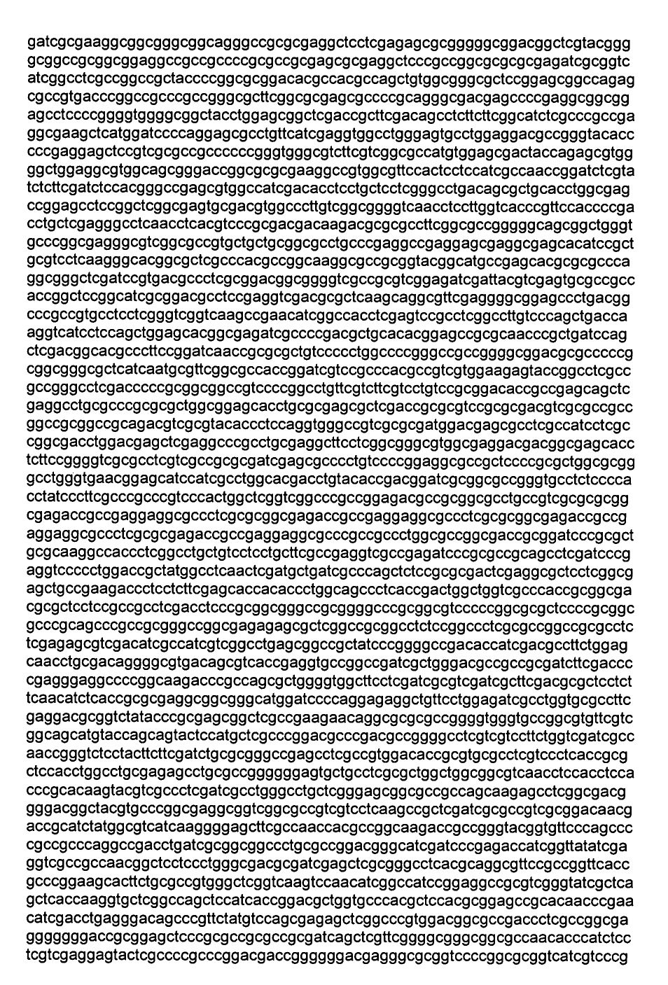

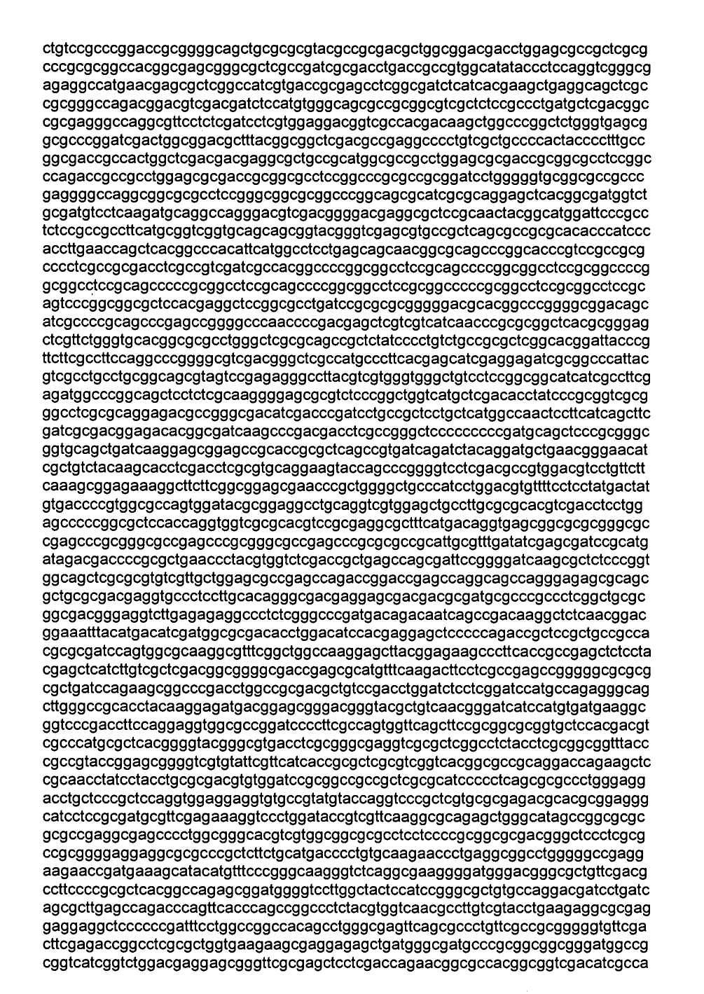

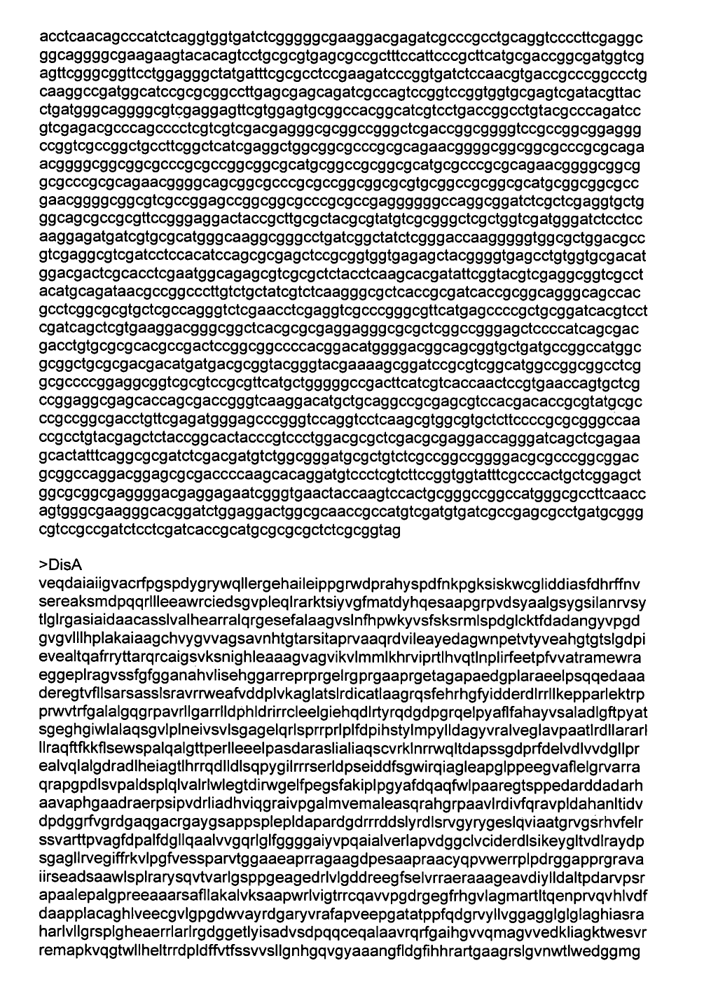

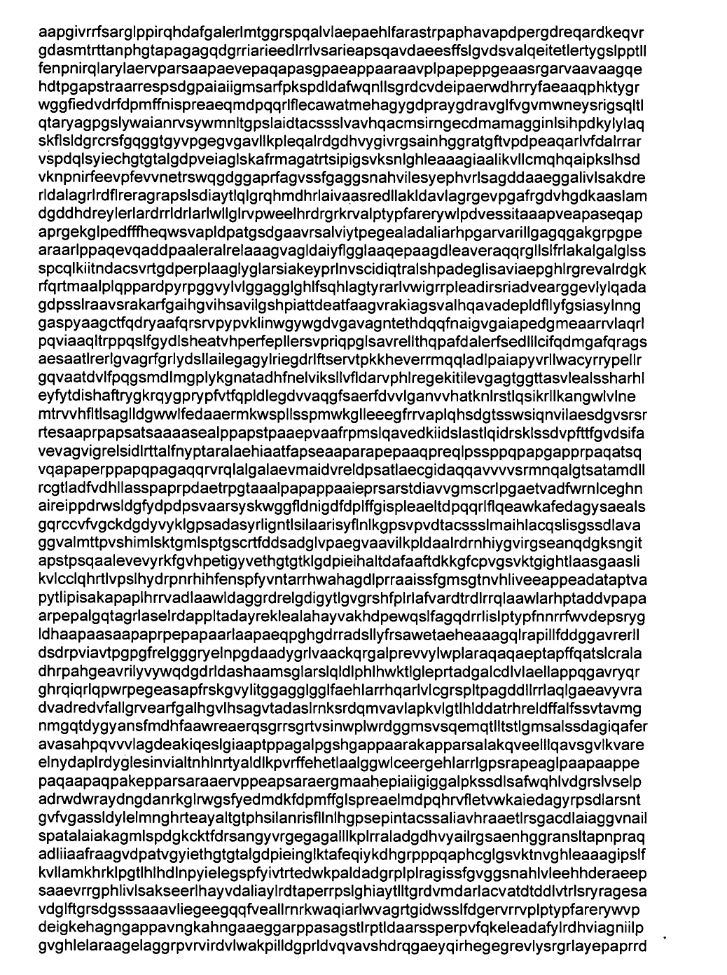

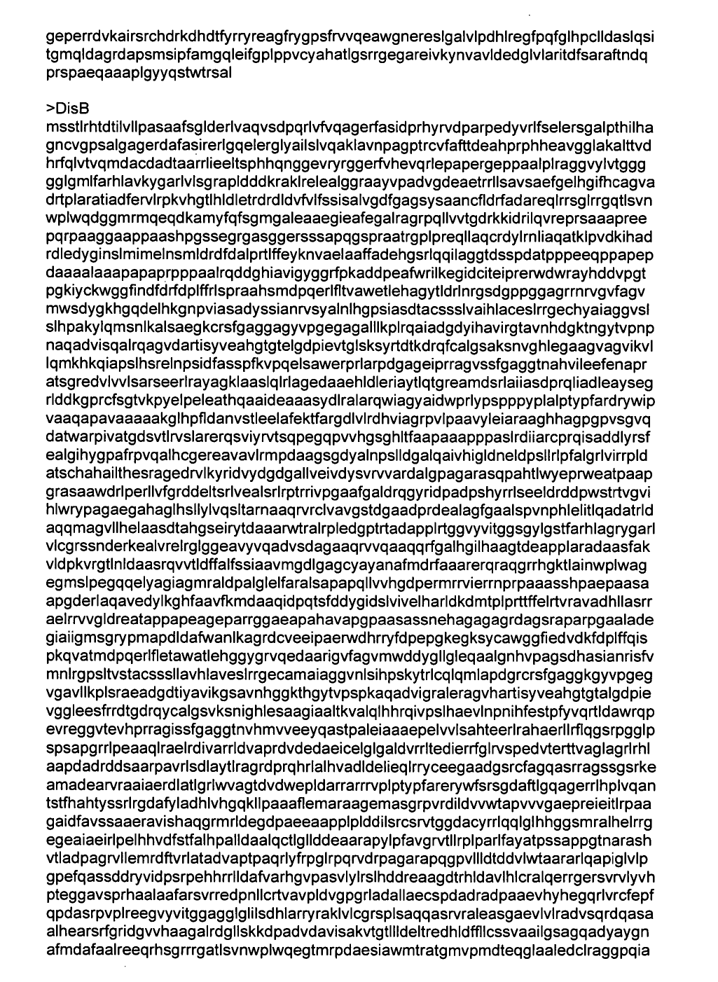

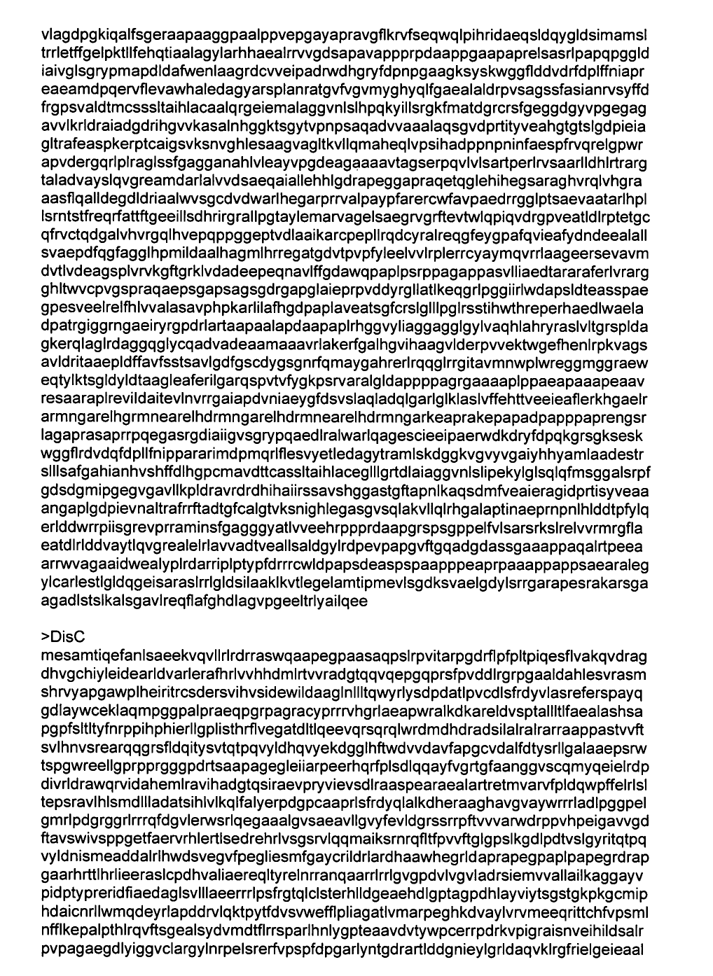

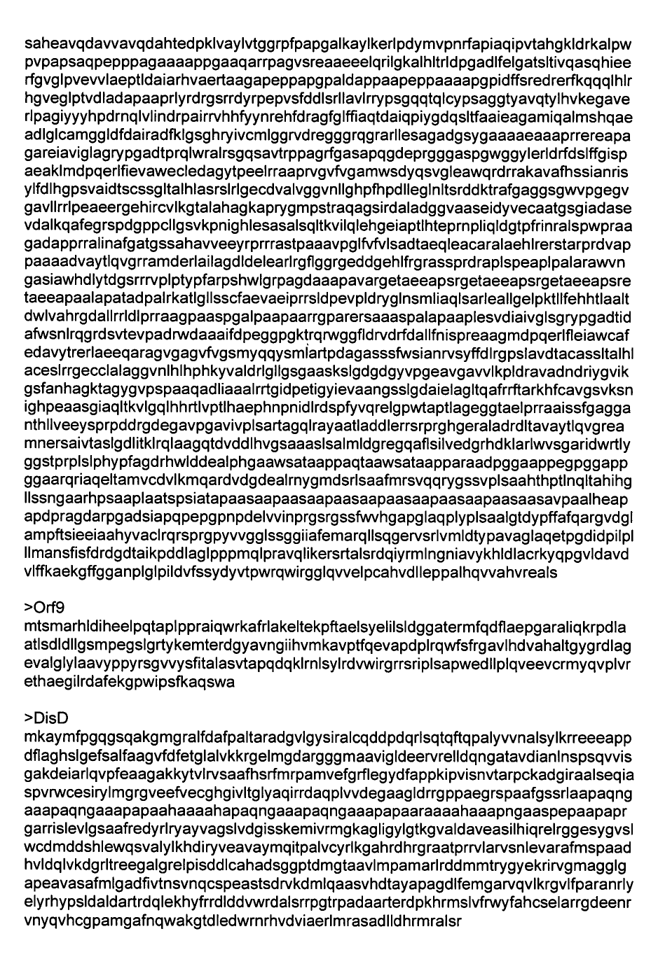

FIG. 3 lists nucleic acid and amino acid sequences relevant to the invention, namely the nucleic acid sequence of pTn-Rec_IE-2 (Seq.-ID No.1), the amino acid sequences of orf 1-pTn-Rec_IE-2 (Seq.-ID No.2), orf 2-pTn-Rec_IE-2 (Seq.-ID No.3), orf 3-pTn-Rec_IE-2 (Seq.-ID No.4), orf 4-pTn-Rec_IE-2 (Seq.-ID No.5), orf 5-pTn-Rec_IE-2 (Seq.-ID No.6), the nucleic acid sequence disA-disD (Seq.-ID No.7) comprising genes disA, disB, disC, orf 9 and disD, and amino acid sequences of DisA (Seq.-ID No.8), DisB (Seq.-ID No.9), DisC (Seq.-ID No.10), orf 9 (Seq.-ID No.11) and DisD (Seq.-ID No.12).

The functions proposed in Table 1 above have been identified by a similarity search on known sequences, however, the gene products from the orfs of Table 1 can differ according to their function in the biosynthetic gene cluster for disorazoles.

An analysis of the genomic DNA region encoding disA through disD has revealed several orfs in the vicinity of disA through disD, summarised in Table 1.

TABLE 1
Orfs identified in the biosynthetic gene cluster for disorazoles

| | | Orientation | Proposed Function of | | Similarity/ | Acc. No. of |
|---|---|---|---|---|---|---|
| Gene | Size (Da/bp) | (strand) | the Similar Protein | Similarity to Source | Identity | similar protein |
| orf1 | 49316/1374 | − | blr4832 Sugar (and | Bradyrhizobium | 49%/67%, | NC_004463.1 |
| | | | other) transporter | japonicum USDA 110 | | |
| orf2 | 51696/1449 | − | probable two-component | Pseudomonas | 49%/66% | NC_002516.1 |
| | | | response regulator, | aeruginosa PAO1 | | |
| | | | signal receiver domain | | | |
| orf3 | 45545/1293 | + | hypothetical protein | Leptospira | 27%/40% | NC_004342.1 |
| | | | | interrogans | | |
| | | | | serovar Lai | | |
| | | | | str. 56601 | | |
| orf4 | 56119/1641 | − | no prediction | | | |
| orf5 | 48994/1371 | − | probable two-component | Pseudomonas | 51%/69% | NC_002516.1 |
| | | | response regulator, | aeruginosa PAO1 | | |
| | | | signal receiver domain | | | |
| orf6 | 105961/3021 | − | sensory box histidine | Pseudomonas | 39%/55% | NC_002947.3 |
| | | | kinase | putida KT2440 | | |
| orf7 | 34954/975 | − | phosphotransferase | Escherichia coli | 29%/40% | Q47395 |
| orf8 | 37435/1053 | + | putative serine/threonine | Streptomyces | 33%/48% | NC_003155.2 |
| | | | protein kinase | avermitilis MA-4680 | | |
| disA-C | | + | | | | |
| orf9 | 30717/822 | + | no functional prediction | | | |
| disD | | + | | | | |
| orf10 | 23476/642 | − | phosphotransferase | Bacillus subtilis | 38%/56% | NC_000964.2 |
| | | | | subsp. subtilis | | |
| | | | | str. 168 | | |
| orf11 | 46773/1287 | + | putative sugar transporter | Streptomyces | 27%/41% | NC_003155.2 |
| | | | | avermitilis MA-4680 | | |
| orf12 | 32992/912 | + | ABC membrane transporter | Brevibacterium | 36%/53% | Q93RD7 |
| | | | homologue | fuscum var. | | |
| | | | | dextranlyticum | | |
| orf13 | 31993/882 | + | ABC membrane transporter | Brevibacterium | 51%/72% | Q93RD6 |
| | | | homologue | fuscum var. | | |
| | | | | dextranlyticum | | |
| orf14 | 86590/2355 | + | putative sugar hydrolase | Streptomyces | 61%/72% | NP_733521 |
| | | | | coelicolor A3(2) | | |
| orf15 | 105005/2892 | + | putative sugar hydrolase | Streptomyces | 46%/59% | NP_629813 |
| | | | | coelicolor A3(2) | | |
| orf16 | 121293/3273 | + | serine-threonine protein | Mycobacterium | 36%/53% | NP_301681 |
| | | | kinase | leprae TN | | |
| orf17 | 23384/642 | − | no prediction | | | |
| orf18 | 35402/999 | − | no prediction | | | |
| orf19 | 25075/657 | − | no prediction | | | |

FIG. 1 schematically depicts the arrangement of genes disA, disB, disC, orf9, and disD, wherein the abbreviations refer to catalytic centers and domains as follows:
Dark shade ( [symbol] ): polyketide synthase (PKS), Light shade ( [symbol] ): nonribosomal protein synthetase (NRPS), KS: ketosynthase, DH: β-hydroxydehydratase, KR: β-ketoacyl reductase, ACP: acyl carrier protein, MT: methyltransferase, HC: heterocyclization domain, A: adenylation domain, PCP: peptidyl carrier protein, Ox: oxidation domain, TE: thioesterase domain,
AT: acyl transferase, Or: oxidoreductase, and ↓: site of insertion of transposon in different mutants.

The sites indicated by the arrows (↓) are designated as Sol2_EX_{—}13-21 and So12_EX_{—}2793, which are So ce 12 mutants from which the plasmids pTn-Rec13-21 and pTn-Rec2793, respectively, were recovered.

The arrangement of genes adjacent to the insertion site of the transposon mutant So ce 12_EXI_IE-2 is schematically depicted in FIG. 2.

For the gene products of disA through disD, functions can be proposed for individual protein domains by homology search. These proposed functions, including their relative positions in the individual nucleic acid sequences are listed in Table 2 below.

TABLE 2
Disorazole biosynthetic genes disA, disB, disC and disD

| | | Proposed Function |
|---|---|---|
| Protein | Size | (Protein domains with their positions |
| (Gene) | (Da/bp) | in the amino acid sequences of FIG. 3) |
| DisA | 647772/ | PKS Domains: KS1 (3-428), DH1 (953-1144), |
| (disA) | 18036 | KR1(1528-1779), ACP1 (1821-1889), KS2 |
| | | (1971-2395); KR2 (2856-3105), MT2 |
| | | (3225-3463), ACP2 (3537-3606), ACP2b |
| | | (3672-3741), KS3 (3779-4201), KR3 |
| | | (4642-4898), ACP3 (4918-4987), KS4 |
| | | (5059-5490), DH4 (5649-5878) |
| DisB | 672408/ | PKS Domains: KR4 (238-492), ACP4 |
| (disB) | 18771 | (547-615), KS5 (676-1114), DH5 |
| | | (1274-1476), KR5 (1836-2093), |
| | | ACP5 (2108-2176), KS6 (2255-2686), |
| | | DH6 (2944-3149), KR6 (3490-3738), |
| | | ACP6 (3776-3824), KS7 (3876-4304), |
| | | DH7 (4472-4679), KR7 (5049-5302), |
| | | ACP7 (5316-5398), KS8 (5500-5926), |
| | | ACP8 (6123-6192) |
| DisC | 409960/ | NRPS Domains: HC1a (58-506), HC1b |
| (disC) | 11379 | (532-955), A1 (1035-1551), PCP1 |
| | | (1580-1647), OX (1649-1836), |
| | | PKS Domains: KS9 (1882-2309), ACP9 |
| | | (2542-2609), KS10 (2668-3098), |
| | | ACP10 (3399-3468), TE (3521-3701) |
| DisD | 90953/ | PKS-Domains: AT (1-280), OX (393-839) |
| (disD) | 2526 | |

Abbreviations are according to FIG. 1.

However, when analysing the synthesis of disorazoles in microorganisms expressing the biosynthetic gene cluster consisting of the sequences encoding DisA, DisB, DisC and DisD only, homologous sequences of which have been described in WO 2004/053065 A2, it is considered impossible that the full range of derivative disorazoles could be produced with the translation products DisA, DisB, DisC and DisD only. The reason is that comparative analysis showed that DisA, DisB, DisC and DisD lack at least some functions, e.g. necessary for hydroxylation, epoxidation and methoxylation, that are assumed necessary for synthesis of at least some known derivatives of disorazole.

Further analysis of the genomic region adjacent the genes disA through disD, for example the gene products of those orfs listed in Table 2 above, did not identify coding sequences for accessory functions to complement the biosynthetic pathway of DisA through DisD to allow production of disorazole or the range of known disorazole derivatives.

Analysis of the two additional disorazole negative mutants revealed further sequences obtainable from Sorangium cellulosum So ce 12, at least one of which encodes a translation product that is necessary for synthesis of disorazoles in combination with the translation products of disA, disB, disC and disD, preferably in combination with the translation product of orf 9. These additional nucleic acid sequences have been identified on recovered plasmids of disorazole negative So ce 12 mutants and are summarised in Table 3 below.

TABLE 3
Recovered plasmids and proposed
function of the encoded proteins

| | | | Identity/ |
|---|---|---|---|
| | | | Similarity |
| | Proposed function | Source of the | (DNA/ |
| Plasmid | of the similar protein | similar protein | protein) |
| pTn-Rec_2793 | BarG (PKS) barbamide | Lyngbya | 39%/57% |
| | biosynthetic gene | majuscula | |
| | cluster | | |
| pTn-Rec_13-3 | 5′ to transposition | Rhodopirellula | 28%/45% |
| | site: no prediction | baltica SH 1 | |
| | 3′ to transposition | | |
| | site: carbamoyltrans- | | |
| | ferase BlmD | | |
| pTn-Rec_13-21 | LnmJ (PKS) leinamycin | Streptomyces | 29%/40% |
| | biosynthetic gene | atroolivaceus | |
| | cluster | | |
| pTn-Rec_IE-2 | beta-lactamase | Oceanobacillus | 38%/53% |
| | putative esterase | iheyensis | 30%/48% |
| | | Rhodopirellula | |
| | | baltica SH 1 | |

The proposed functions have been identified by similarity searches with known proteins but may be different from the proposed functions indicated here according to their functions within the biosynthetic gene pathway.

Sequencing of pTn-Rec_IE-2 identified a total of 5 orfs and their putative functions, which are summarized in Table 4 below:

TABLE 4
Proteins encoded on the plasmid pTn-RecIE-2 and their putative function

| | Position on DNA | | | | Similarity/ |
|---|---|---|---|---|---|
| | sequence of | | Proposed Function of | | Identity |
| orf | pTn-RecIE-2 | Size (Da/bp) | the Similar Protein | Source | (DNA/protein) |
| orf 1- | 58-579 | 18008/522 | arylesterase- | Caulobacter | 29%/43% |
| pTn-RecIE-2 | | | related protein | crescentus | |
| orf 2- | 1665-2255 | 20979/591 | SAM-dependent | Gloeobacter | 48%/58% |
| pTn-RecIE-2 | | | methyl-transferase | violaceus | |
| orf 3- | 3159-4442 | 46369/1284 | putative esterase | Rhodopirellula | 35%/51% |
| pTn-RecIE-2 | | | beta-lactamase | baltica SH 1 | |
| | | | | Oceanobacillus | |
| | | | | iheyensis | |
| orf 4- | 4459-6240 | 62063/1782 | adenylate cyclase 2 | Stigmatella | 31%/51% |
| pTn-RecIE-2 | | | | aurantiaca | |
| orf 5- | 6328-7181 | 29564/854 | outer membrane | Myxococcus | 36%/46 |
| pTn-RecIE-2 | | | protein (incomplete) | xanthus | |

In a first embodiment of the present invention, at least one of the translation products of Table 4 is used in combination with the translation products of disA through disD to provide the biosynthetic pathway for disorazoles, in a preferred embodiment, at least 2, more preferred three or four translation products of the sequences identified in Table 4 participate in the biosynthetic pathway for disorazoles in combination with disA through disD, preferably including the translation product of orf 9.

The DNA sequences of disA, disB, disC, disD and orf 1-pTn-Rec_IE-2, orf 2-pTn-Rec_IE-2, orf 3-pTn-Rec_IE-2, orf 4-pTn-Rec_IE-2, and orf 5-pTn-Rec_IE-2 as well as their translation products obtained from Sorangium cellulosum So ce 12 are listed in FIG. 3. These specific sequences are preferred for performing the present invention, but other coding sequences and peptides derivable therefrom providing the respective activity necessary in the disorazole synthetic pathway are also applicable in the present invention and can replace the sequences of FIG. 3.

The present invention will now be described in greater detail by way of examples, which are not intended to limit the scope of the invention.

### EXAMPLE 1

### Cloning and Sequencing of Nucleic Acid Sequences Complementing the Biosynthetic Pathway Enzymes for Disorazoles

Nucleic acid sequences, the translation products of which participate in the biosynthetic pathway for disorazoles have been identified using a transposon recovery procedure from disorazole negative transposon mutants of Sorangium cellulosum strain So ce 12. Strain So ce 12 is available at NCIMB Aberdeen, UK, under accession No. NCIB 12134.

For transposon mutagenesis, transposon termed pMiniHimarHyg which is applicable to myxobacteria was used, comprising the hygromycin resistance, but lacking the genes for conjugational DNA transfer. The transformation of Sorangium cellulosum was obtained by electroporation as described in European patent application EP 04 103 546.0, filed on 23 Jul. 2004 with the European patent office.

Disorazole negative mutants were detected in a bioassay using an overlay with the disorazole sensitive yeast R. glutinis. In this bioassay, transposon mutants were plated on PM 12 agar plates without hygromycin at 32° C. until colonies became visible, then overlayed with R. glutinis, incubated overnight at 30° C. and growth inhibition zones were compared to a wild type Sorangium cellulosum So ce 12.

Transposon recovery from disorazole negative transposon mutant colonies was essentially carried out as described in Kopp et al. (J. Biotech 107, 29 (2004))

### EXAMPLE 2

### Heterologous Expression of Biosynthetic Pathway Enzymes for the Production of Disorazole

The core biosynthetic gene cluster and their respective translation products sufficient for the biosynthesis of disorazoles was determined by heterologous gene expression experiments. As expected, the core enzymes comprising disA, disB, disC as well as disD are regarded as necessary components for the biosynthetic pathway. An optional and preferably included component is orf 9.

The core cluster comprising disA, disB, disC as well as disD needs complementation with at least an expression cassette encoding orf 3-pTn-Rec_IE-2, optionally in combination with orf 1-pTn-Rec_IE-2, optionally in combination with orf 2-pTn-Rec_IE-2, optionally in combination with orf 4-pTn-Rec_IE-2, and optionally in combination with orf 5-pTn-Rec_IE-2.

When expressing sequences encoding at least one, preferably two, more preferably three or four and most preferably all of the group comprising orf 1-pTn-Rec_IE-2, orf 2-pTn-Rec_IE-2, orf 4-pTn-Rec_IE-2, and orf 5-pTn-Rec_IE-2, in combination with orf 3 -pTn-Rec_IE-2 to supplement the expression cassettes encoding disA-disD, optionally orf 9, respectively, production of disorazoles was found.

The number of derivative disorazoles varied according to the sequences selected among orf 1-pTn-Rec_IE-2, orf 2-pTn-Rec_IE-2, orf 4-pTn-Rec_IE-2, and orf 5-pTn-Rec_IE-2 for expression in combination with orf 3-pTn-Rec_IE-2 and disA-disD, optionally orf 9. It is preferred that the coding sequences are contained intra-chromosomally in their natural arrangement.

For production of disorazoles, the identification of the set of genes or gene cluster according to the invention allows to modify producer strains, for example by specifically targeted modification of regulatory elements, e.g. the introduction of stronger promoters for disA, disB, disC, orf 9, and/or disD, and/or for the complementing genes orf 1-pTn-Rec_IE-2, orf 2-pTn-Rec_IE-2, orf 3-pTn-Rec_IE-2, orf 4-pTn-Rec_IE-2, and/or orf 5-pTn-Rec_IE-2.

Alternatively, heterologous expression can be employed using microorganisms which are no natural producers of disorazole. For heterologous expression, Myxococcales, preferably Myxococcus xanthus, or Polyangium, also termed Sorangium, e. g. Sorangium cellulosum accessible as ATCC 25531, ATCC 29479 (DSMZ 2044), Stigmatella aurantiaca, Angiococcus disciformis and strains of the genus Pseudomonas, e.g. Pseudomonas putida, Pseudomonas stutzeri, and Pseudomonas syringae can be used.

Alternatively, the expression products, i. e. proteins derivable from the aforementioned sets of genes for the synthetic pathway, can be used in an extracellular synthesis system, e. g. as catalysts like an immobilized enzyme system for synthesis of disorazoles.

1. Proteins for the synthesis of a polyketide, said proteins having the activity of translation products encoded by the genes disA, disB, disC and disD obtainable from Sorangium cellulosum in combination with a translation product encoded by orf 3-pTnRec_IE-2, obtainable from Sorangium cellulosum.

2. Proteins according to claim 1, comprising the activity of at least one translation product encoded by one of orf 1-pTnRec_IE-2, orf 2-pTnRec_IE-2, orf 4-pTnRec_IE-2, and orf 5-pTnRec_IE-2, obtainable from Sorangium cellulosum.

3. Proteins according to claim 1, wherein the polyketide is disorazole A1 or a derivative thereof.

4. Nucleic acid sequence, encoding a protein according to claim 1.

5. Genetically manipulated microorganism, comprising nucleic acid sequences encoding proteins according to claim 1.

6. Genetically manipulated microorganism according to claim 5, selected from Myxococcales, Sorangium or Pseudomonas.

7. Process for producing polyketides, comprising using proteins according to claim 1.

8. Process for producing polyketides, comprising using a nucleic acid sequence according to claim 4.

9. Process for producing polyketides, comprising using a microorganism according to claim 5.


SEQUENCE LISTING (12 sequences)

SEQ ID NO: 1 - DNA, 7181 bp, Sorangium cellulosum
acgcgtcgggatagcccttaaccaatgtgacgctggcgtgcgcggtggtctcgggacctacttcgccggcgcagcggcctgagcggcgcgcttgcgccccttggcgctcggcttctttaccggcgccggccccgccgccgtcttccgcgcgttcgcgctcgcctgctcggcagtcttcgcgtcgcccgcatagcgggcgaaccgccgatcgcccccctggaaaatccgcttctccagcttgagctccttcagcgctgctgcggcgcgcgtctgcgggatgcccgcctgcttggcgacctcgctcgcgctcaggcccgagctcgctttgacgatgcgctcgaccgcctccagcgtctcctgccgctccgccgaggcggcgggcgacttgcgcgggcgaccaggccggcgcttcggcgcggcgccacgcttggccgcgacggaagacggtttggactggaccgctccagggcgggcgcgcgaacggctgcggcgcggcgcaggaggaggaacgagcggcgtttctcccgtgacagcgcccagctgattcttgacgagcgcgaggatcgcctcgtccggcatcttccgcacgaggtttacaatctgctgtgaaaaggcatccgttgccatgtgctctccctcgtcgccgaatccagctcggcggttggatacgactatgccgttgccacttgcctggtcaagcgaattgaggatagcgggccgtcttttctttcattaggcgctgcgggcgggctcaatcggagagctcattggacggccacgaacggctgggctgctttgctcgacaggcgccgagccacgaaacgccccgggagcctcgattgatagccgagacccctcccccgcgcaggacggcccgccccgcgccaatccgcgctcggatcccccacgatgcactgcggcaatgggtgtctcgcgggctcgcgggctggtcgggtaagccctgtatgaggccgagcggccccgctccttcatcgagcccgtcgcgggcggcaatgcgggcgcgcgcacaactgggaggtgcaccggcccccggccagccgcctccccgcaaccattgagaagcgccccgggcccggcgcgagcgcctggcgaccacacacacgcgaccagcgagtggctacatcacacgccctgggcgcacggctggcctatgtaggaccgcgataggacgcggcctgggcaggagagcgcagcgccgtcgccggcctcgatgtcccgcgcgcggccgcgcccgcgcgagctgcgggggctctcgtccttttggtcgcgcccggcggaccgtccacgcgcgcgcctcttcttcttcgtggcaggagctctcgttacatcgcgacgcgctgacgagggctcgtggccctcggcgatctcgctccggcgctcccggggagagcggagcctccggcctggcgctctcgagcgtgcgcggcccgctcccccggcgcgcccgccctccacgacgccggcgcctgctccaatgagccgagcaccgcgggcccggggccggcccgctccgcctcgacttcttcaaacgccttcctgtccatatccagatccaggatgcagcggacccagccgccagccgagattggtcagccgccgacaatcggttgacacgggcgtcgcccacggagaaggatgcgagcatgtcgctggcgcgcgtgctcgagccggaggtcatggataccgacgaggacgcgcgcgcctacgacagcatggaccacgccgccgtcaacacagccttctgcgacgatctcctctcccaggggcccgatctcgcatacgccctcgacgtcggcacggggacgtgcctgttgcccatcgagctttgcaggcgcgtgccgtcggcgcggatcaaggcgatcgatctctcggcgtcgatgctcgcgctcgggcgcagccacgtcgagcaggccgggctcaccggcgccgtcacgctcgcgctcgcggacgccaaggcgctgcccgagccggacaagacgttttccgctgtcctctcgaacagcatcgtccatcacatcccggagcccgaggcggtgctcggcgagatgctccgcgtcctccggcccggcgggctcctgttcgtgcgagacctggttcgcccggacgacgaggcgtcgctcagcgccctcgtcgatacgtatgcagcgaacgacacgccgtaccagcgggccctcttcgacgcgtcgcgttgtactggggcgcagatgccggtgatccctgaagagttctcctgatatagagctgtgagatgatgcgttgcgttatttgacggactcatcaggagagtcgatgttggcgccctgttgctgcgcggccaccgtggcagcaaccacggtgcacacgaccggatacgcacgtggcgtcgatgcaggctgggcggacgaggcgcgctgggccgcccgcgcgacgcgcggcgctgctgcgtgtgcgcctgcgcaccgccggcgcgaggcgatgagccgcgggcacctgggaggtccgcgggtatcgtgctctcgatgcctccgacccaatcttcaccgcgtgagggacaagcgctgtcctgacgagtggatgggcccgagaggatgagcgcggcggagggtgggcgcgagccgcgcggtgaacgcccctcgaacgagctcgcgccgacgtctcctctctcgcgatgacgtgcaggccggcgcggcgcctcgcgggtcggagttgcccgcggcgaacgggcaccccgggcgcggccatcgcgcgtggccgcgctcaccgctcgcgctcaccgctcgcgctcaccggcggcgcggaggcgcggaggcgccgtctccaggcgaggccccggcgggcggcgcatgcgtccacggtcgtccttggtcgtcctggatgtcgaggtgagcgcgcatcggcttgcggcatcccgcaatcgattgcgcgtcgattgccgctgtgccaactcaatcggacgaaccgcgcccgtgctacggaccgaaaaggcagcggacatcaggtcttgttcgcggtggaggatgagggggggagagccggcgcagctggcgtacaaattgccgttctcagcgacagacgcccacgctgaatgcgccccggaattccccgtgagtgccccgtggaacgaatcgtcttcgatgaaagcagggttgagaagcgccatgaccgaccaacgacgagcgttcaaccgcaggcagttcttgcaaggagtgagtcagttcgccgtggtggccaccgcggcccccgcgctcggcttcatgggcgccgggagcggcgtcaccacgaccacgctcttgccgggtcggcccgaggacgtgggcatgtgctcggatcggctggaggacgtcttcgcgcggatccagcgccgcgtgaacgacggcatgttccccggcgccacggccctggtcgcgcggcgcggggtcgtcgtggggcaccgggccttcggcagcaaggtccgcggcgccgacgagccggtcacgctcgatacgatcttcgacctgcagtcgatgaccaaggtgctctcgacggcggcgtcggcgctcgcgctcgtgcaggagggcgtgatccggctgagcacccgggtcgccagcgtcctgccggagttcgccgcgaacggaaagagggacatcacggtctatgacatgctccggtactcggccggcctgccgatggacaaccagctcctcgacgagccggacgacgagacggtctggcgctgtatggccgagacgccgctcgagtacacgccgggcacgtccgtgctctacagcgacctcacgtaccgcctcctcgggcgcatgctcgcggccgccgcgggcgaagatctcgataccgtcgcggccacgcgcatctggggccccctcggcatgaacgatacgatgtacaacccgcccgcggagctccacgagcgcgtggcggccacgggctactcgacccgccgcggctacgtggtccggggcgaggtgcaggacgagcaggacttcgcgctgggcggcgtcaccggctgcgacggcgtcttctccacggcgacggacgtcgccgtcttctgccagatggtgctgaacggcggcgcctacggcggtgtcgaggtcctgcggcccgatctcgcccgccgcatgaggaagaacaatacgcctcaggtgaccgaggcggccaccgacacctcgccgatcaacaacctgatactcgcccccaagggcctcggctgggagatgtggacgcggcgcttctcgaacggcggcatgcgcctgtcgccgcgctcctacggcaaggcgggcggcgccggcaccttcatgtgggtcgatccggaccgcgagatcatcggcgtcctcctgaccaaccacgggttgccccagccgttcgacgagctcgggtgggaccggatgctcgactgcgtgggctgcgccgagttcttcgacggcatcatcaacgcggtcaagtggtgagcgccgcgccgcggcctcagcccgcgtcgagcgcgtagacggccgtggccgtctttcgccccgtcaggtcgcgctcgcccagcgcgcgcagcgggaaggcgtcgccggcgagggcccgggtcgccccggagaccagcacctggttgggcgccgcgatcgcctcgagccgcgacgccacgttcaccacgtcgccgatcaccgtgtactccatccgcttgtcggagccgatgttgccgacgatggcctctcccgagttcacgccgacgccgaggcggatctcgatatcatacctctcgcgccactcctcgttggccgtctcgaggaagcgcatcatgtcctccgcggccgcgagcgcgcggcgcgcgtgatcctcctgcgcgagcggggcgccccacaccgccatcacgcagtcgccgatgaacttgtcgaccgtgccgccgtggcggaacacgatctcggtcagcatcgagaagagctcgttcaggagggcgaccatccgctcggcgtcgcgcgactcggcgagcggggtgaagcccacgacgtcggcgaagagcaccgagacggccgcgcgcttgccgccgagggcgagcgggtgctcgccccgcacgatggcgtcgacgaccgcccggtccatgaaccggccgaggtcgccgcggagcttcgcctcgcgcgcgatgtcggcctcgccgcgctccaggtcctcggccatgcggcggagcgagcgcgtgagctcgccgatctcgtcgcggcgctcgtgctgcgcgccgccgagcgcgagctcccgccagcgccgggcgccgacgagccgcgcctgcgcgacgaggcgcaggatcgggcgcgtcacgtggccggccgtgagggcggcgagggcgagcgcgaggaccagggcgccggcgccgaccgcgaggccgcggcgccgcatctcgtcgaggaccgcgaaggccacaggctccgggcgccagatcgcgaccgcccagccgaggtcgggcagcgtctcgatcgcgccgatcatgcgctggccgccctcgtcgtgcggggcgagcacgctgatcctgtcggtccacacggcgcccggcgggagcttgccccagagcggcaaggcggagacgtcgctgcccggcgcggcgccgggcacgccgtacgccgcgatcgcccggcgatcgtggcccgcgatgaggaggcgcgcctcgccgccgccgaggcggcgcccggcgatggccgcgaggtcgcgctccagcgcgtcgagcccgacctccgcgaccacgtagccggccggaccgcgcgacggcgccggccccgcgccggccgcggcgccggggggccgcacccgcgccacgaggagcccgcgggcaggcccgaccacgccgaagccgacgccgcgctcgtcggcgagctcgcgcagcgcgggcggcgccgccggcacgtccgcgccggacgacgccttgcggatcagggtgctgatcccggcgtcgggcacctccagccgcacggcgtcgatgagcgcgcgcgtcgcgacgagggcgcgcaccgacgcgacagcgtcctcgtcccggatggcgccggccgcggcgtggccgaagatggccgcggccgcgaccgcgtcgctctcgacgtcgcggacgagccgggtcgaggccgaggcgacctcggagaggatcgccgcctgcaagctctcctcggacgtccggacggcgcgccggttcacgtcggcgaggagcgcgacggcgacggccacgggcgcgagggcgatcgcggtgaagagcgcgagcgccttggtcttgagcgagagcgccctcacgcgcgcgcctccggccgcggcgaggcccggcggaggccgcgcgcggccgggcgcgcgggggcggcgcgcgggcgggcagggcgggcgtcagtcaacggggccgtccttccagccggcggcgccctcgtccgtgcgcttgcggatctggaactccacccggcggttcctggcctggccctcgcgggtgcggttgtcggcgatggggcggcgcgcgccgaagccgcgcgcctcgaggcggcgggcgtcgatgccgttggcggtcagccacgcgacaacggcgagcgcgcggcgctgcgagaggaccaggttcgcccggtcggtgccctggttgtccgtgtgtccgtcgaccgcgacccgggcgatctcggggtgctccgcgaggagcgccgcgacctcggcgagcagctcgaagctctccggctcgatctccgccgtgccgcgcgggaactggacctgctgcaggatgacgatctgcgagccctcgatgcgcaccgcgaaggggcagccgtgcgtcgcgcggtcctcgctcggcttgccgcgctcgtaggggcaggcgtcggccgcgttggggatgccgtcgccgtccgagtccgcggggcagccgtgcttcggcgtgggtcgggaccccgcgttcgcgggtgcctcggtcgccggtcgggaccccgcgttcgcgggtgcctcggtcgccggccgggaccccgcgttcgcgggtgcctcggtcgccggccgggaccccgcgttcgcgggtgcctcggtcgccggccgggaccccgcgtccgcggtcacgcccgcgctgctcacgcccggcacgtccgggcagagatcgtcggcgtcgtagatcccgtcgtcgtcgcgatccggcgggcagccgcgccggaacgccggccgggcccccgccgcggctggcgggcgcgcctcgccgatgacccggggacacgcgt

SEQ ID NO: 2 - protein, 173 aa, Sorangium cellulosum
MPDEAILALVKNQLGAVTGETPLVPPPAPRRSRSRARPGAVQSKPSSVAAKRGAAPKRRPGRPRKSPAASAERQETLEAVERIVKASSGLSASEVAKQAGIPQTRAAAALKELKLEKRIFQGGDRRFARYAGDAKTAEQASANARKTAAGPAPVKKPSAKGRKRAAQAAAPAK

SEQ ID NO: 3 - protein, 196 aa, Sorangium cellulosum
MSLARVLEPEVMDTDEDARAYDSMDHAAVNTAFCDDLLSQGPDLAYALDVGTGTCLLPIELCRRVPSARIKAIDLSASMLALGRSHVEQAGLTGAVTLALADAKALPEPDKTFSAVLSNSIVHHIPEPEAVLGEMLRVLRPGGLLFVRDLVRPDDEASLSALVDTYAANDTPYQRALFDASRCTGAQMPVIPEEFS

SEQ ID NO: 4 - protein, 427 aa, Sorangium cellulosum
MTDQRRAFNRRQFLQGVSQFAVVATAAPALGFMGAGSGVTTTTLLPGRPEDVGMCSDRLEDVFARIQRRVNDGMFPGATALVARRGVVVGHRAFGSKVRGADEPVTLDTIFDLQSMTKVLSTAASALALVQEGVIRLSTRVASVLPEFAANGKRDITVYDMLRYSAGLPMDNQLLDEPDDETVWRCMAETPLEYTPGTSVLYSDLTYRLLGRMLAAAAGEDLDTVAATRIWGPLGMNDTMYNPPAELHERVAATGYSTRRGYVVRGEVQDEQDFALGGVTGCDGVFSTATDVAVFCQMVLNGGAYGGVEVLRPDLARRMRKNNTPQVTEAATDTSPINNLILAPKGLGWEMWTRRFSNGGMRLSPRSYGKAGGAGTFMWVDPDREIIGVLLTNHGLPQPFDELGWDRMLDCVGCAEFFDGIINAVKW

SEQ ID NO: 5 - protein, 593 aa, Sorangium cellulosum
VRALSLKTKALALFTAIALAPVAVAVALLADVNRRAVRTSEESLQAAILSEVASASTRLVRDVESDAVAAAAIFGHAAAGAIRDEDAVASVRALVATRALIDAVRLEVPDAGISTLIRKASSGADVPAAPPALRELADERGVGFGVVGPARGLLVARVRPPGAAAGAGPAPSRGPAGYVVAEVGLDALERDLAAIAGRRLGGGEARLLIAGHDRRAIAAYGVPGAAPGSDVSALPLWGKLPPGAVWTDRISVLAPHDEGGQRMIGAIETLPDLGWAVAIWRPEPVAFAVLDEMRRRGLAVGAGALVLALALAALTAGHVTRPILRLVAQARLVGARRWRELALGGAQHERRDEIGELTRSLRRMAEDLERGEADIAREAKLRGDLGRFMDRAVVDAIVRGEHPLALGGKRAAVSVLFADVVGFTPLAESRDAERMVALLNELFSMLTEIVFRHGGTVDKFIGDCVMAVWGAPLAQEDHARRALAAAEDMMRFLETANEEWRERYDIEIRLGVGVNSGEAIVGNIGSDKRMEYTVIGDVVNVASRLEAIAAPNQVLVSGATRALAGDAFPLRALGERDLTGRKTATAVYALDAG

SEQ ID NO: 6 - protein, 283 aa, Sorangium cellulosum
ACPRVIGEARPPAAAGARPAFRRGCPPDRDDDGIYDADDLCPDVPGVSSAGVTADAGSRPATEAPANAGSRPATEAPANAGSRPATEAPANAGSRPATEAPANAGSRPTPKHGCPADSDGDGIPNAADACPYERGKPSEDRATHGCPFAVRIEGSQIVILQQVQFPRGTAEIEPESFELLAEVAALLAEHPEIARVAVDGHTDNQGTDRANLVLSQRRALAVVAWLTANGIDARRLEARGFGARRPIADNRTREGQARNRRVEFQIRKRTDEGAAGWKDGPVD

SEQ ID NO: 7 - DNA, 52125 bp, Sorangium cellulosum
gtggagcaggacgccattgcgatcatcggcgtagcgtgccgatttcccgggtctccggactatggccggtactggcagctgctcgagcggggcgagcatgccatcctcgagatcccacccggccggtgggatccccgggcccattattcccctgacttcaataagcctggcaagagcatcagcaagtggtgcgggctgatagacgacatcgccagcttcgaccaccgcttcttcaacgtgtcggagcgcgaggcgaagagcatggaccctcagcagcgcctgctcctggaagaggcatggcgctgcatcgaggactccggcgtgccgctcgagcagctccgcgcccggaagacgtccatctacgtgggcttcatggcgacggattaccaccaggagtccgcggccccgggccgcccggtcgacagctacgccgccctggggagctacggctccatcctggccaaccgcgtctcctatacgctcgggctgcgcggcgcgagcatcgccatcgacgccgcctgcgcctcctccctcgtcgcgctccacgaggccaggcgcgctctccagcgaggtgaaagcgaatttgcgctcgccgccggcgtgagcctcaactttcatccttggaagtacgtctccttctccaagtcgcgcatgctcagcccggacgggctgtgcaagacgttcgacgcggacgcgaacggctacgtccccggagacggggtgggtgtcctcttgctgcaccccctggccaaggccatcgctgcgggatgccacgtctacggcgtcgtcgcgggctccgcggtcaaccacaccggcaccgcgcgttccatcaccgcgccgcgcgtcgccgcccagcgggacgtcatcctcgaggcctacgaggacgcgggctggaacccggagacggtgacgtacgtggaggcccatggcaccggcacctcgctgggggaccccatcgaggtggaagcgctgacccaggcgttccgccgctacacgaccgcgcgccagcgctgcgcgatcgggtcggtgaaatcgaacatcggccacctcgaggcagccgcgggcgtcgctggggtcatcaaggtgctcatgatgctgaagcaccgcgtgatcccgcggacgctgcatgtccagacgctcaacccgctcatccgcttcgaggagacgcccttcgtggtcgccacccgcgccatggaatggcgcgcggaaggaggcgagcccctgcgcgcaggggtgagctcgttcggcttcggtggcgccaacgcccacgtcctgatatccgagcacggcggcgcgcgccgcgagccccgcccgcgaggcgagctccgcggcccccgcggcgcagccccgcggggcgagacggcgggcgctccagcggaggacggcccgctggcccgcgcggaggagctcccttcgcagcaggaggacgccgcggcggacgagcgcgaaggcaccgtcttcctcctctccgccaggtccgcgtcgagcctgtcgagggccgtccgacgctgggaggccttcgtcgacgatcccctcgtgaaggcaggcctggccacctcgctccgcgatatctgcgcgaccctggccgccggacggcaaagcttcgagcaccgccacggcttctacatcgacgacgagcgagacctccggcgcttgctcaaggaaccgccggcgcgcctggagaagacccgacctcctcgctgggtgacgcggttcggcgcgctcgccctcgggcagggcaggcccgccgtccgtctgctcggcgcgcgccgcctgctcgatcctcaccttgaccgcatccggaggtgcctcgaggagctggggatcgagcaccaggatctccggacgtaccgtcaggacggcgatcccgggcgccaggagctgccctatgcgttcctcttcgctcacgcgtacgtctcggcgctcgcggacctcggcttcacgccgtacgcgaccagcggagagggtcacggcatctggttggcgctcgcccagagcggggtcttgccgctgaacgagatcgtgtcggtgctctcgggggccggagagctccagaggctctcgccccggcgtcccaggctgccgctcttcgatcccatccattccacctacctgatgccgtacctcctggacgcgggctacgtccgcgcgctcgtggagggcctggcggttccggcagcgacgctccgtgacctcctcgcgagggctcgactcctgctccgcgcgcagttcaccttcaagaagttcctgagcgagtggtcgccggccttgcaggccctgggcacgacgcctgagcgcctgctcgaggaggagctccccgcgtccgacgctcgcgcctcgctgatcgcgctcatcgcgcagagctgcgtgcgcaagctgaaccgcaggtggcagctcacggacgcgccctcctcgggagatccgcggttcgacgagctcgtcgacctggtggtcgacgggctcctgccgcgcgaggcgctcgtgcagctcgccctcggcgatcgggcggacctccacgagatcgccggcaccctgcaccggcgtcaggacctgctcgatctcagccagccgtacggcatcctgcggaggcgcagcgagcgcctggacccgagcgagatcgacgatttttccggctggatccggcagatcgcgggcctcgaagcgccgggcctgccgcccgaagagggcgtcgcgttcctggagctcggcagggtggcgaggcgcgcgcagcgggcgccggggccagatctgagcgtcccagcgctggacagcccgctgcagctcgtcgcgctgcgcctgtggctggaggggactgacatccggtggggagagctctttccggaggggagcttcgcgaagatcccgctgcctggctatgcgttcgatcaggcgcagttctggctgccggcagccagagaaggcacgtcccctcccgaggacgcgcgcgacgacgccgacgcgcgacacgccgccgtcgcgccgcacggcgcggcggaccgggctgaacgcccctcgatccccgtggaccgcctgatcgccgatcacgtcatccagggccgcgccatcgtgcccggcgccctcatggtcgagatggccctggaggcgtcacagcgcgcccacgggcggccggcggcggtcctgagagacatcgtgttccagcgggcagttccgctcgacgcgcacgcgaacctcacgatcgatgtcgaccctgacggcgggcgtttcgtggggagagacggcgcgcagggggcatgccgtggagcctacgggagcgcgcccccctctccgctggagcccctcgatgcgccggcccgcgacggcgaccgccgccgcgacgatagcctctaccgcgacctttcgcgcgtcgggtaccgctacggcgagagcctgcaggtgatcgccgcgaccggtcgggtcggctcgcgccatgtgttcgagctccgctccagcgtcgctcgcacgacgcctgtcgccggcttcgacccagcgctcttcgacgggctgctccaggcggcgctggtcgtggggcagcgcctcgggctgttcggcggaggcggcgcgatctatgtgcctcaagccatcgcgctcgtcgagcggctcgctccggtggacgggggctgcctggtctgcatcgacgagcgcgatctctcgatcaaggagtacggcctgaccgtcgacctgcgcgcctacgatccgtcgggggccggcctgctccgggtagagggcatcttctttcgaaaggtgctgccgggcttcgtcgagagctcccctgccagggtgaccggcggcgccgcggaggcgccacgccgcgccggagcggccggagatcccgagtcggccgcgccgcgagcagcgtgctatcagcccgtctgggagcgacggccgctcccggatcgcggcggggcacccccgcgtggtcgcgcggtggcgatcatccgctccgaggcggactccgcagcctggctctcgcccctgcgagcgcgctattcacaggtcacggtggcgcgcctcggcagcccgccgggtgaggcgggcgaagatcggctcgtcctgggcgacgatcgagaggagggcttctccgagctggtgcgccgggcggagagagcggccgccggcgaggccgtcgacatctacctcctggacgcgctgacgcccgacgcccgcgtcccctcgcgcgcgcctgcggcgctcgagccggcgctgggcccccgcgaagaggccgcggcgcgcagcgcgttcctgctggccaaggccctggtgaagagcgcggcgccgtggcgcctggtcatcggcacgcggcgctgccaggccgtcgtgcccggagaccggggcgaagggttccgccacggggtgctcgccggcatggcccggaccctgacgcaggagaacccgcgggttcaggtccacctggtggatttcgacgccgctcctccactcgcatgcgccggccacctcgtcgaggagtgcggtgtgctcggcccgggggactgggtagcctaccgcgacggcgcccgttacgtccgcgcctttgcgccggtcgaggagcccggcgcgacggccacgccgccgttccaggacggtcgcgtctatctgctggtcggtggcgccggcgggctcggcctcggcctcgcggggcacatcgcctcccgggcgcatgcgcgcctggtcctgctcggccgctctccgctcggccacgaggcggagcgccgcctggcccgcctgcgcggcgacggcggcgagactctctacatcagcgcagatgtcagcgatccacagcagtgcgagcaggccctggcggcggtccgccagcgattcggcgccatccacggcgtggtgcagatggccggcgtggtcgaggacaagctgatcgcaggcaagacctgggagtcggtccgacgagagatggcgcccaaggtgcaggggacctggctattgcacgagctcacccggcgcgaccctctcgacttcttcgtgaccttctcctccgtcgtctccctgctgggaaaccacggccaggtgggctacgcagcggccaacgggttcctcgacggcttcatccaccaccgggcccgcaccggcgccgcgggcaggagcctcggcgtgaactggacgttgtgggaggacggcggcatgggcgcggctcccgggatcgtgcgccggttctcggcgcgcgggctccctcccatccggcagcacgacgccttcggcgcgctcgaacggttgatgaccggcggacggtcgccgcaggcgctcgtcctcgcagagcccgcagagcacctcttcgcgagagcttctacacgacctgctccccacgcggtcgctcccgatccggagcgcggcgatcgcgagcaggcccgagacaaggaacaggttcggggagacgcgagcatgacacgtactacggctaatcctcacgggacggcgcctgcaggggcaggacaggacgggcggcgtatcgcccggatcgaggaggatctccggcggctcgtctccgccaggatcgaggctccgtcgcaagcggtcgacgcggaagagtccttcttttcgctcggggtcgactccgtggctcttcaagagatcacggagacgctcgagcgcacctacggctccctgccgccgacgctgctcttcgagaatccgaacatccgccagctggcgcggtacctcgcggagcgcgtccccgcgaggtcggcagcccccgcggaggtggagccggcgcaggcgcccgccagcgggcccgcagaggcgccgcctgccgcccgagcggccgtgcccctccccgcgccggagccgcctggcgaggccgcctcccgcggcgcgcgggtggctgccgtcgcggccggccaggagcacgacacgccgggcgcgccctccacccgcgccgcgcgccgcgagagcccgtccgatggccctgcgatcgcgatcatcggcatgagcgcccgcttccccaagtcccccgatctggacgcgttctggcagaacctgctctcgggccgggattgcgtcgacgagatccccgccgagcgctgggaccaccggcgctacttcgccgaggcggcgcagccccacaagacgtacgggcggtggggcgggttcatcgaggacgtcgaccgcttcgacccgatgttcttcaacatctccccgcgcgaggcggagcagatggatccacagcagcgcctcttcctggagtgcgcgtgggcgacgatggagcacgcgggatacggcgacccgcgcgcgtacggcgaccgcgccgtggggttgttcgtcggggtgatgtggaacgaatacagccgcatcggcagccagctcaccctgcagaccgcgcgctacgcggggccgggctcgctctactgggccatcgccaaccgggtctcgtactggatgaacctcaccggtccgagcctggccatcgatacggcctgctcttcctcgctggtcgccgtccatcaggcctgcatgagcattcgcaacggagagtgcgacatggccatggccggcgggatcaacctctcgatccaccccgacaagtacctctacctggcgcagtcgaagttcttgtcgctcgacgggcgctgccgcagcttcggccagggtggcaccggctacgtgcccggcgagggcgtcggcgccgtcctcctcaagccgctggagcaggcgctgcgtgacggcgatcacgtctacggcatcgtgcgcggctccgcgatcaaccacggcggccgcgccaccggcttcacggtccccgatccggaagcccaggcgaggctcgtgttcgacgccctgcgacgcgcgcgcgtgtcccccgatcagctgagctacatcgagtgccacggcacgggcacggcgctcggagatcccgtcgagatcgccggtctcagcaaggcgttccgcatggcgggcgccacccgcacgagcatccccatcggctccgtcaaatccaacctgggccacctggaggccgccgcggggatcgccgcgctcatcaaggtcctcctgtgcatgcagcaccaggcgatcccgaagagcctgcacagcgacgtcaagaaccccaacatccgcttcgaggaggtcccgttcgaggtcgtgaacgagacgcgctcgtggcagggggacggcggggcgccccgctttgccggcgtgagctccttcggcgcgggcggctccaacgcccatgtcatcctcgagtcgtacgagcctcatgtgcgcctcagcgcgggcgacgacgccgcggagggaggagccctcatcgtgctgtccgcgaaggaccgcgagcgcctcgacgccctcgcgggacggctgagggatttcctgcgcgagcgggcaggccgcgccccctcgctgagcgacatcgcctacacgctgcagctggggcgccagcacatggatcatcggctggcgatcgtcgccgccagccgggaggatctgctggccaagctggacgccgtgctcgctggccgcggcgaggtgcccggcgcgttccggggcgatgtccacggcgacaaggcggcttccctcgccatggacggggacgatcatgaccgcgagtacctggagaggctcgcccgcgaccgcaggctggacaggctcgctcgcctctggctgctggggctcagggtcccgtgggaggagctccaccgagatcgcggccgcaagcgggtcgccctgcccacgtaccccttcgcccgcgagcgttactggctgcctgacgtggagagctcgatcaccgccgcggcgccggtcgaggcccccgcgtcggagcaggcccccgcgccccggggggagaagggccttccggaagacttcttcttccacgagcaatggtccgtggcgccgctggatcctgcgacgggctcggacggcgctgcggtccggtccgcgctcgtgatctacacgccggagggtgaagcgctcgccgacgcgctgatcgcgaggcaccccggcgctcgcgtcgcccgtattctcctcggcgccggccagggggcgaaggggcgccccggcccggaggcccgcgccgctcggcttccccccgcgcaggaggttcaggccgacgatcctgccgccctcgagcgcgccctccgcgagctggccgccgccggcgtcgcgggcctcgacgccatctacttcctcggcggtctggccgcacaggagcccgcggcgggcgacctggaggccgtggagcgcgcccagcagcgtgggctgctctcgctgtttcgcctggcgaaggcgctgggcgccctgggcctttcgtcgtcgccctgccagctgaagatcatcaccaacgatgcttgctcggtgcggaccggagatcccgagcgcccgctcgccgcgggcctgtacggcctggctcgatccatcgccaaggagtacccgcgcctcaacgtcagctgcatcgacatccagactcgagcgctgagccacccggccgatgaggggctcatcagcgcggtgatcgccgagccaggtcacctccgcggccgagaggtggcgctgcgggacggcaagcgcttccagcgcacgatggccgccttgccgctgcagccgccggcgagggatccttaccgtccaggcggcgtgtacctggtccttggcggcgccggtgggctcggccacctgttcagccagcacctcgcagggacctaccgcgctcggctcgtgtggatcggccggcgccccctcgaggccgacatccggtcgcgcatcgccgacgtcgaggcgcgcggaggcgaggtcctctatctccaggccgacgccggcgacccgagctccctgcgcgctgccgtctcccgcgccaaggcgcgcttcggcgcgatccacggggtcatccactccgcggtcatcctcgggagccaccccatcgccaccaccgacgaggccacgttcgccgccggagtccgcgccaagatcgccggcagcgtcgcgctccaccaggcggtcgccgacgagcccctcgatttcttgctctatttcggatccatcgcctcctacctcaacaacggcggggccagcccgtacgccgccggctgcacgttccaggacaggtacgcggcattccagcgttcccgcgtgccctacccggtcaagctcatcaactgggggtactggggcgacgtcggcgcggtcgccggcaacaccgagactcatgaccagcagttcaacgccatcggcgtcggggccatcgcgcccgaggacgggatggaggcggcgcgccgcgtcctcgcgcagcgcctgccccaggtgatcgcggcgcagctcacgcgcccgccccaaagcctcttcggctacgacctgagccacgaggcgaccgtccacccggagcgcttcgagccgctgctcgagcggagcgtgccgcgcatccagcccggcctcagcgcggtccgcgagctcctgacgcatcagcccgcgttcgacgcgctggagcgcttcagcgaggatctgctgctctgcatcttccaggacatgggcgcgttccagcgcgccggcagcgcggaatcggcggcgaccctgcgagaacggctgggcgtcgcgggccgcttcggccggctctacgactccctgctcgcgatcctcgagggggccggttacctgcgcatcgaaggagatcggctgttcacgagcgaacgggtgacgccaaagaagcacgaggtggaacggcggatgcagcagctggcggatctgccggcgatcgcgccgtacgtccgcttgctctgggcgtgctatcggcggtaccccgagctgctccgcggtcaggtagccgcgacggacgtgctcttcccgcagggctcgatggatctgatggggccgctctacaagggcaacgccacggccgaccatttcaacgagctggtcatcaagagcctcctcgtgttcctggacgcccgcgtcccgcacctgcgagagggcgagaagatcacgatcctggaggtaggggctgggacgggcggcaccaccgcgtccgtgctcgaggcgctctcctcccatgcgcgccacctcgagtacttctataccgacatctctcacgccttcacgcgatacggcaagcgccagtatggcccgcgctaccccttcgtcaccttccagcccctcgacctcgagggggacgtggtggcgcagggcttctccgcagagcgcttcgacgtggtgctgggcgcgaacgtcgtgcacgcgacaaagaacctgcgcagcacgctgcagagcatcaagcggctcctcaaggcgaacggctggctcgtcctgaacgagatgacccgcgtcgttcacttcctcacgctctctgcgggtctcctggacggctggtggctcttcgaagacgccgccgagcgcatgaaatggtcccctctgctcagctccccgatgtggaagggcctgctggaggaagagggattccgccgggtcgctcctctccagcacagcgacggcacgtcctcctggtcgatccagaacgtgatcctcgccgagagcgacggcgtgagccgaagccggcggaccgagagcgccgctccgcggccagcgccgtcggccacgagcgcggcggcggcgtccgaagcgctcccgcccgccccgtccacccccgccgccgagccggtcgccgcgttccggccgatgtccctgcaggccgtcgaggacaagatcatcgatagcctcgcgagcacgctgcagatcgacaggtccaagctcagctcggacgtgccattcacgacgttcggggtcgattcgatcttcgccgtggaggtcgccggcgtgatcgggcgcgagctgagcatcgatctcaggaccacggccctgttcaactatcccaccgcgcgcgcgctcgccgagcacatcgccgcgacgttcgcccccagcgaggcggccccggccagagcgcccgaaccggcggcgcagccgcgggagcagctcccctcgagcccgccgcagccggcgccgggagcgccgccgcggccagcgcaggccacgtcgcaggtccaggcgccggcgccggagcgtccgccggcgccgcagccggccggcgcccagcagcgggtccggcagctcgccctgggtgccctcgccgaggtgatggcgatcgacgtgagggagctcgatccgagcgcgaccctcgccgagtgcggcatcgacgctcagcaggccgtcgtggtggtgagccgcatgaaccaggccctcgggacgagcgccaccgccatggatctcctccgatgcgggaccctcgcggacttcgtggaccacctcctcgcgtcctcgcccgcgccgcgcccggacgcggagacccgccccggcaccgccgcggcgctcccggcgcccgcgccccctgcggcgatcgagcccaggtccgcccggagcacggacatcgcggtggtgggcatgtcctgccggctgccgggcgccgagacggtcgccgacttctggcggaatctctgcgagggtcataacgccatacgggagatcccgcctgaccgctggtccctcgatgggttctacgatcccgaccccagcgtcgctgcccgcagctacagcaagtggggtgggtttctcgacaacatcggcgacttcgacccgctcttcttcggcatctcaccgctggaggcggagctcacggatccgcaacaacgcctctttctccaggaggcctggaaggcgttcgaggacgccgggtacagcgccgaggcgctgagcgggcagcggtgctgcgtgttcgtggggtgcaaggacggggattacgtctacaagctcggcccgtcggcggacgcctcctaccggctcatcgggaacaccctgtccatcctcgcggcccgcatctcctattttctcaacctcaaggggccgagcgtccctgtcgacaccgcttgctcttcctccttgatggcgatccacctggcctgccagagcctgatcagcgggtccagcgacctcgccgtggccgggggcgtcgccctgatgaccacgccggtgagccacatcatgctcagcaagacggggatgctgtcgcccacggggagctgccgcacgttcgacgactccgccgatgggctggtccccgccgagggggtggccgccgtcatcctgaagccgctcgacgccgccctgcgcgatcgcaaccacatctacggggtgatccgcggctccgaggcgaaccaggacggcaagagcaacggcatcacggcgcccagcaccccctcgcaggccgccctggaggtcgaggtctaccgcaagttcggggttcacccggagaccatcggctacgtcgagacccacggcaccggcaccaagctgggggaccccatcgagatccacgcgctcacggacgcgttcgccgccttcaccgacaagaaggggttctgcccggtcgggtccgtgaagacggggatcggccacacgctggcagcgtccggggccgcctccctcatcaaggtgctctgctgcctccagcaccgcacgctcgtgccgtcgctccactatgaccggcccaacaggcacatccacttcgagaacagcccgttctacgtcaacaccgcccggaggcactgggcgcacgccggcgatctcccgcgccgggcggcgatcagctcgttcggcatgagcggcaccaacgtgcacctcatcgtcgaggaggcgcctccggaggccgacgccaccgcgcccacggtggccccctataccctcatcccgatctcggcgaaggcgccggcgccgctccatcgcagggtggcggatctggccgcctggctcgacgccggcgggcgcgaccgcgagctgggcgatatcgggtacaccctgggcgtcggccggagccattttcccctgcggctcgccttcgtcgcgcgcgacacgcgcgacctgcgccgccagctggcggcgtggctcgcgcgccacccgaccgcggacgacgtgccggcgccggccgcgcggccggagcccgcgctcggccagacggcgggccgcctggcgagcgagctccgcgacgcgcccccgctcaccgccgacgcgtaccgtgagaagctggaagccctggcccacgcctatgtggcaaagcacgatcctgagtggcagtccctgttcgcgggtcaggatcgacgcctgatctcgctgcccacgtacccgttcaacaaccgccggttctgggtggacgagccctcgcggtacgggctcgatcacgccgcgccggccgccagcgcggcgccggcgccgcggccggagcccgcgccggccgcgcgcctcgcggcgccggcggagcagccggggcacggagaccggcgagcagattcgctcctttatttcagatcggcctgggaaaccgcagagcacgaggctgccgcgggccagctccgcgctccgatcctgctcttcgacgacggcggcgccgtgcgcgagcggctgctggacagcgaccgccccgtcatcgccgtcacgccgggccccgggttccgcgagctgggaggcggccgctacgagctgaaccccggcgacgcggcggattacggccgcctcgtcgccgcctgcaagcagcggggcgcgctgccgcgcgaggtcgtgtacctgtggccgctcgcgcgagctcaggcgcaggcggagccgacggcgcccttcttccaggcgacctctctgtgccgcgcgctcgccgaccatcgccccgcgcacggcgaggctgtccgcatcctgtacgtctactggcaggacggggatcggctggacgccagccatgcagccatgagcggcctggcccgcagcctgcagctcgaccttccgcacctccactggaagacgctcggcctcgagccgcggaccgccgacggcgcgctgtgcgatctcgtcctcgccgagctgctcgccccgccgcagggcgcggtccgctaccagcgggggcaccggcagatccagcggctccagccgtggcgccccgagggcgaggcgagcgcgcccttccgcagcaaaggggtctatctgatcaccggcggcgccggtgggctgggcggcctgttcgccgagcacctcgctcgccgccatcaagccaggctggtcctgtgcgggcgctctcccttgacgccggccggcgacgacctcctccgccgcctcgcccagctcggcgcggaggcggtctatgtgcgggccgacgtcgccgatcgcgaggacgtgttcgcgctgctcgggcgcgtcgaggcccggttcggcgcgctccacggcgtcctccacagcgccggcgtcaccgccgacgcgagcttgcgcaacaagagccgtgaccagatggtcgccgtgctcgcgccgaaggtgctcggcaccctgcacctcgacgacgccacccgccatcgagagctggatttctttgccctgttctcctccgtcaccgcggtcatgggcaacatggggcagacggactacggctacgccaacagcttcatggaccacttcgcggcctggcgcgaggccgagcggcagagcggacgccgcagcggaaggaccgtgtcgatcaactggccgctctggcgagacggcgggatgagcgtctcgcaagagatgcagacgctgctcacgtccaccctcggcatgagcgcgctctcgagcgacgcgggcatccaggccttcgagcgcgccgtggcctcggcgcacccccaggtcgtggtcctcgccggtgacgaggccaagatccaggagagcctcggcatcgcggccccgaccccgcccgccggcgcgctcccggggtcgcacggcgcccctcccgcggctcgcgcgaaggcgccccccgcgcgcagcgcgctggcaaagcaggtcgaggagctcctgctgcaggcggtctccggggtgttgaaggtcgctcgcgaagagctgaattacgatgcgccgctgagagattacgggctggagtccatcaacgtcatcgccctcaccaaccatctgaaccggacctacgcgctcgacctcaagccggtgcggttcttcgagcacgagacgctcgccgcgctgggcggttggctatgcgaggagcgcggggagcacctggctcgacgcttgggcccctcgcgcgcgcccgaggccgggctccccgctgcccccgcggcgccccccgagcccgcgcaggccgccccggcgcagccggcgaaggagcccccggcacggagcgcgcgggccgccgagcgcgtcccgccggaggcgccctcggcccgggctgaacgggggatggcggcccacgagcccatcgccatcatcggtatcggcggggccctgccgaagtccagcgacctgagcgcgttctggcagcacctcgtggacggccgctccctcgtctccgagctgcccgccgatcgctgggactggcgtgcttacgacaacggcgacgcgaatcggaaggggctgcgctgggggagcttctacgaggacatggataagttcgatccgatgttcttcgggctctccccgcgggaggccgagctgatggatccccagcaccgcgtcttcctcgagaccgtgtggaaggccatcgaggacgccggatacaggccctccgatctggcgaggagcaacaccggcgtcttcgtcggcgcgtcgtcgctcgactatctcgagctgatgaacggacaccggacggaggcgtacgccctcaccggcacgccgcactcgatcctggcgaaccggatctcgttcttgctgaacctgcacgggcccagcgagcccatcaacaccgcctgctcgagcgcgctgatcgccgtccaccgcgccgcggagaccctccgcagcggcgcctgcgatctggccatcgccggcggggtcaacgcgatcctcagccccgcgacggccctggccatcgcgaaggcaggcatgctgagcccggacgggaagtgcaagaccttcgatcggagcgcgaacggctacgtccgcggcgagggggccggcgcgctgctcctcaagccgctccgccgcgcgctcgccgacggggatcacgtctatgcgatcctgcgcggcagcgccgagaaccacggcgggcgcgccaactcgctcaccgcccccaacccgcgggcccaggcggatctcatcatcgcggccttccgcgcggcgggcgtcgatccggccaccgtgggctacatcgagacccacggcacgggcaccgccctcggcgatcccatcgagatcaacggcctcaagacggccttcgagcagatctacaaggatcatggccggccgccgccgcaggcgccgcactgcgggctcggctcggtcaagaccaacgtcggccacctggaggcggccgccgggatcccgagcctcttcaaggtcctcttggcgatgaagcaccgcaagctgcccgggactctccacctccacgacctgaacccctacatcgagctcgagggcagccccttctacatcgtcaccaggacggaggactggaagcccgctctggacgccgacggccgccccctcccgctgcgcgccgggatcagctccttcggcgtcggcggctccaacgcccacctggtcctcgaggagcaccacgacgagcgcgccgaggagccgtccgcggccgaggtccggcgcggccctcatctgatcgtcctctccgcgaagagcgaggagcgcctccacgcgtatgtagacgcgttgatcgcctacctccgcgacacggcgccggagcgccggccgtccctcgggcacatcgcgtataccctgctcaccggtcgtgacgtcatggacgcccgcctcgcctgcgtggcgaccgacacggacgacctcgtcacccggctctcccgttaccgggccggcgagagcgcggtggacgggctgttcaccggtcggagcgacgggagctccagcgcggcggccgtgctcatcgagggcgaagagggccagcagttcgtcgaggcgctcctccgcaaccgcaagtgggcccagatcgctcgcctgtgggtcgccgggcgcacggggatcgactggtcctctctgttcgacggcgagcgcgtgcggcgcgtgccgctgccgacctaccccttcgcgcgggagcgatactgggtgcctgacgagatcggcaaggagcacgccgggaacggcgcgccgcccgccgtcaacggcaaggcgcacaacggtgccgccgagggcggcgcccgtcccccggccagcgcggggagcacgctgcgcccgacgctcgacgctgcgcgctcgagccccgagcggcccgtcttccagaaggagctggaggccgacgccttttatctgagagatcacgtcatcgccggcaacatcatccttccgggcgtggggcacctggagctcgctcgcgcggccggtgagctcgccggcggacgaccggtgcgcgtcatccgggacgtcctgtgggcaaagcccatcctgctcgacggaccgcggctcgatgtgcaggtggcggtcagccatgaccgtcagggcgccgagtaccagatccgccacgagggcgagggccgcgaggtcctctactcgcgcggaaggctggcctacgagccggctccgcgccgcgacggcgagccggagcgccgcgacgtgaaggcgatacggtctcgatgccacgaccgcaaagatcacgacacgttctaccgccggtatcgagaagccgggttccggtacggcccctccttccgggtcgtccaggaggcctgggggaacgagcgcgagtccttgggagcgctcgtcctgccagaccacctgcgcgaggggttcccgcagttcggcctgcacccctgcctgctggacgcctccctgcaatccatcaccgggatgcagctcgacgccggccgcgacgcgccctccatgagcatccccttcgccatgggccagctggagatcttcggcccgctgcctcccgtgtgctacgcgcacgcgaccctgggctcgcgccgcggcgaaggggcgcgcgagatcgtcaagtacaacgtcgcggtcctcgacgaggacggcctcgtgctggcgcgcatcacggacttcagcgcgcgcgccttcacgaacgaccagccgcgcagcccagccgagcaggccgctgcgccgctcggctattaccaatcgacctggaccagaagcgcgctttgaacgtcggggtaacctcatgtccagcactctccgccacacagacaccatcctcgtcctgctgcccgcgagcgcggcgttcagcgggctcgacgagcgcctggtcgcgcaggtatccgatccgcaacgcctcgtcttcgtgcaggccggcgagcgcttcgcctcgatcgatccgcgacattaccgcgtcgatccggcgcgcccggaggattacgtccggctgttctcggagctcgagcgcagcggcgcgctgcccacgcatatcctccacgcgggcaactgcgtcggcccgagcgcgctgggggctggcgagcgcgacgcgttcgcgagcatccgcgagcggctaggccaggagctggagcgcggcctgtacgcgatcctctcgctggtccaagccaagctggccgtcaaccccgctggccccacccgctgcgtgttcgcgttcacgaccgacgaggcccacccgcgcccgcaccacgaggcggtgggcggcctggcaaaggccctcacgacggtcgatcatcgcttccagctcgtcaccgtgcagatggacgcgtgcgacgcggacaccgcggcgcgccgcctcatcgaggagctgacctcgcctcaccaccagaatggcggcgaggtgcgctacaggggcggggagcggttcgtacacgaggtgcagcggctggagcccgcgcccgagcggggagagccgccggccgcgctcccgctgcgcgccggcggcgtgtacctcgtgaccggcggcggcggcggcctggggatgctgttcgcccggcacctggccgtgaagtacggcgcccgcctggtcctcagcggccgcgctccgctcgacgacgacaagcgcgcgaagctccgcgagctcgaggcgctcggcggccgcgcggcgtacgtgcccgcggacgtgggcgacgaggccgagacgcggcgcctgctctccgccgtctccgcggagttcggcgagctccacggcatcttccactgcgctggagtggccgatcgcacgccgctcgcgagggccacgatcgcagatttcgagagggtattgcgccccaaggtgcacggcacgctccacctcgacctggagacccgcgaccgcgatctcgacgtcttcgtcctgttctcgtcgatctcggcgctggtcggcgacttcggcgcgggcagctactccgcggcgaactgcttcctcgatcgcttcgccgacgcccgcgagcagctgcgacgcagcggcctgcgccgcggccagaccctgtcggtcaactggcccctctggcaggacgggggcatgaggatgcaagagcaggacaaggccatgtacttccagttctccggcatgggggccctggaagcggccgagggcatcgaggccttcgagggcgccctccgggccgggcggccccagctgctcgtggtcaccggcgaccgcaagaagatcgaccgcatcctgcaggttcgcgagccgcgctcggcggccgctccacgcgaagagccgcagcggcccgccgccggaggcgccgcgccgccggccgcgagccatccggggtcgagcgagggccgaggcgcctccgggggagagcggtccagctcagcgccgcagggctcgccgcgcgcagcgacgcgaggcccgctgccacgagagcagctcctcgcgcagtgcagagactacctgcgcaatctgatcgcccaagccacaaagctccccgtcgacaagatccacgcggacagggatctggaggactacggcatcaactccctcatgatcatggagctcaactccatgctcgacagggatttcgacgcgctgccgcgcaccctcttcttcgagtacaagaacgtcgccgagctcgccgccttcttcgccgacgagcacgggtcgcggctgcagcagatcctcgcggggggcacggactcgagcccggacgcgacgccgccccctgaggagcagccgccggcgccggagcccgacgcggcggccgccctcgcggcagcgccggcgcccgctccgcgcccgccgcccgcagcgctccgtcaggacgacgggcacatcgccgtgatcgggtacggcggccgcttccctaaggcggacgatcccgaggcgttctggaggatcctcaaggaggggatcgactgcatcacggagatcccccgcgagcggtgggactggcgcgcgtaccacgacgacgtcccggggacgccggggaagatctattgcaagtggggcggcttcatcaacgacttcgaccgcttcgatccgctcttcttccgcctctctccgcgcgcggcgcacagcatggatccgcaggagcggctgttcctgacggtcgcctgggagaccctggagcacgctggctacaccctcgatcgcctgaaccgcgggtccgacgggccccccggcggcgcgggccgccgcaaccgggtcggcgtcttcgcgggcgtcatgtggagcgactacggcaagcacgggcaggacgagctccacaagggaaaccccgtgatcgcgagcgccgattactcgtcgatcgccaaccgggtgtcctacgcgctcaacctgcacggccccagcatcgcctccgacacggcctgctcgtcgtcgctcgtcgccatccacctggcctgcgagagcctccggcgaggcgagtgccactacgccatcgccggcggggtgagcctctcgttgcaccccgccaagtacctccagatgagcaacctgaaggccctgtccgccgagggcaagtgccgcagcttcggcgccgggggcgccgggtacgtgcccggcgagggcgcgggcgcgctcctcctcaagccgctgcgtcaggccatcgccgacggcgactacatccacgccgtcatcaggggcaccgcggtcaaccacgacggcaagaccaacgggtacacggtcccgaacccgaacgcgcaagccgacgtcatctctcaggcgctgcggcaggccggcgtcgacgcgcgcacgatcagctacgtggaggcccacgggacaggcaccgagcttggcgatccgatcgaggtgaccggcctgtccaagagctaccggaccgacaccaaggacaggcagttctgcgcgctgggatctgcgaagtccaacgtcggccacctggaaggcgcggccggggtcgccggcgtgatcaaggtgctcttgcagatgaagcacaagcagatcgctccgtcgctgcattcgcgggagctgaaccccagcatcgatttcgcgagctcgcccttcaaggtccctcaggagctcagcgcctgggagcgaccgcgcctcgcgcggccggacggcgcaggagagatcccgcgacgggcgggcgtcagctccttcggcgccggcgggacgaacgcgcacgtcatcctggaggagttcgagaacgcgccgcgcgcgacatcgggtcgggaggacgtcctcgtggtgctctcggccaggagcgaggagcgcctgcgcgcctacgcgggcaagctcgccgcgtccttgcagctgcggctcgccggcgaggacgccgccgagcacctcgacctcgagcgcatcgcctacacgctgcagaccgggcgtgaggcgatggattcgcggctcgccatcatcgcctccgatcctcgacagctcatcgccgacctggaggcctacagcgaaggccgcctggacgacaagggccctcgctgcttctccggcacggtcaagccctatgagctgccggagctcgaggcgacgcaccaggccgccatcgacgaggccgcggcgagctacgacctgcgcgcgctcgcgcgacagtggatcgccggatacgcgatcgactggccgaggctctatccgtctccgccgccctacccgctggccctccccacgtatcccttcgcgcgagaccgctactggatccccgtcgccgcgcaggcgccggcggtcgccgcggcggcggcgaagggcctccaccccttcctggacgccaacgtatccaccctggaggagctggcgttcgagaagaccttcgcccgcggcgacctcgtgctgcgagaccacgtgatcgccggtcgtccggtgctccccgcggcggtgtacctggagatagcccgcgccgccggtcaccacgcagggccggggccggtctccggcgtccaagacgccacgtgggcgaggcccatcgtggccacgggcgactcggtcaccttgcgcgtcagcctcgcccgggagcgccagtctgtcatttaccgtgtcacctcgcagcccgaagggcagccggtggtgcacgggtccgggcacctcaccttcgcggcgcccgccgccgcccccccgccggcgtcgctccgcgacatcatcgcccgctgcccgcggcagatctcggccgacgacctttatcgctccttcgaggcgctggggatccactatggccccgcgttccgccccgttcaggcgctccactgcggggagcgagaggccgtcgccgtcctgaggatgcccgatgccgcgggcagcggcgactacgccctgaacccctcgctgctggacggcgccctgcaggcgatcgtccatatcgggctcgacaacgagctcgatccgtcgctcctgcgcctgcccttcgccctcggccggctcgtgatccggcggcccctcgacgcgacgagctgccacgcgcacgcgatcctcacgcacgagtcgcgcgcaggcgaagaccgggtgctgaaataccgcatcgacgtctatgacggcgacggcgctctccttgtcgagatcgtggactacagcgtacgcgtcgtggcgcgcgacgcgctcggccccgccggcgcccgggcttcgcaacccgcgcacacgctctggtacgagccgcgctgggaggcgacgcccgccgctccggggcgcgcgtccgcggcgtgggatcggctgcccgagcggctcctggtcttcggccgagacgacgagctcacgtcgcgccttgtcgaggcgctgagccggctccggcccacgcggcggatcgtcccgggcgcggcgttcggcgcgctcgaccggcaaggctaccggatcgatccggcggatccgagccactaccgccgcctctcggaggagctggatcgcgacgacccgtggtcgacgaggaccgtaggcgtcattcacctctggcgctatccggccggcgccgagggcgctcacgcagggctccactccctgctctacctcgtccagagcctcaccgcccgcaacgccgcccagcgcgtccggtgcctcgtcgccgtcggatccacggacggcgccgccgatccgcgcgacgaggcgctggccggcttcggcgccgccctgtcccctgtcaaccctcacctcgagctcatcaccctgcaagccgacgcgacgcggctcgacgcgcagcagatggcgggcgtcctgctccacgagctggccgcgtccgacaccgcccatggcagcgagatccgctataccgacgctgctgcccggtggacacgcgcgttacggcccctggaggacgggccgacgcggacagcggacgcgccgccgctgcggacgggcggtgtgtacgtgatcaccggcgggagcggctacctgggctcgaccttcgcgcgccacctcgccgggcggtacggggcgcggctcgtcctctgcggtcgatcctcgaacgacgagcgcaaggaagccctggtgcgcgagctccgcggcctcggtggagaggcggtctatgttcaagcggacgtcagcgacgcaggcgccgcgcagagggtggtgcaggccgcgcagcagcgcttcggggcgctccacggcatcctccacgccgccgggaccgacgaggcgccgccgctcgcgcgcgccgacgccgcctccttcgccaaggtcctggaccccaaggtgcgcgggacgctgaacctggacgccgcgagccgccaggtggtcaccctggacttcttcgcgctcttctcgtcgatcgccgcggtgatgggcgacctcggcgccggctgctacgcgtacgccaacgcgttcatggaccgcttcgccgccgctcgagagcggcagcgcgcgcaaggtcgacgacacggcaagacgctggcgatcaactggcccctgtgggccggcgagggcatgagcctgcccgaagggcagcaggagctgtacgccggcatcgcaggcatgcgcgcgctcgatccggcgctgggcctcgagctcttcgcgcgggccctctcagccccggcgccgcagctgctcgtggtccacggggatcccgagcggatgcggcgggtcatcgagcggaggaacccgcgcccggcggcggcttcatcgcatcccgccgagcccgccgccagcgccgcccccggtgacgagcgcctcgcccaggccgtcgaggattatctcaagggccacttcgccgcggtcttcaagatggacgcggcgcagatcgacccgcaaaccagctttgacgactacggcatcgactcgctcgtgatcgtggagctccacgcgcgcctcgacaaggacatgacgccgctgccgcgcacgacgttcttcgagctccggaccgtccgcgcggtcgccgaccacctgctcgcgtctcgccgcgccgagctgcgccgggtcgtgggcctcgaccgggaggccacggcgccccccgcgccggaggccggcgagcccgcccggcgtggaggcgcggaggcccccgcccacgcggtggccccgggcccggcggccagcgcctcatcgaacgagcacgcgggcgccggagcgggccgcgacgccggcagccgagcgcccgcccggcccggagcggccctcgcggacgaaggcatcgcgatcatcggcatgagcggccggtaccccatggcgcccgacctggacgcgttctgggccaacctcaaggccgggcgcgactgcgtcgaggagatccccgcggagcgatgggaccaccggcggtacttcgaccccgagcccgggaaggagggcaagagctattgcgcgtggggtgggttcatcgaggacgtcgacaagttcgatccgctcttcttccagatctcgcccaagcaggtggcgacgatggacccgcaggagcggctcttcctggagaccgcgtgggccacgctcgagcacggcgggtacgggcgcgtgcaggaagacgcggcccggataggggtgttcgcgggcgtgatgtgggacgattacggcctgctcgggctcgagcaggcggcgctcgggaaccacgtgcccgccggctccgatcacgcctcgatcgcgaaccggatctcgttcgtgatgaacctgagaggcccgagcctcacggtctccacggcgtgctcctcgtcgctcctggcggtgcacctggcggtggagagcctgaggcgaggcgagtgcgccatggccatcgcgggaggcgtcaacctgtccatccacccgagcaagtacacccgtctgtgccagctccagatgctcgcgccggacgggcgctgccgcagcttcggcgccggcggaaaggggtacgtgcccggagagggcgtgggcgccgtgctgctgaagcccctgagcagggccgaggccgacggcgacaccatctacgccgtgatcaagggcagcgccgtcaaccacgggggcaagacccacggatacacggtcccgagccccaaggctcaggccgacgtcatcgggcgcgccctcgagcgcgccggcgtccacgcgcgcacgatcagctacgtggaggcccacggcacgggcaccgcgctgggagatcccatcgaggtcggcgggctggaggagagcttcaggcgcgacaccggcgacaggcagtactgcgcgctgggctcggtgaaatccaacatcggccacctcgagagcgccgcagggatcgcggccctcacgaaggtcgcgctgcagctgcaccaccggcagatcgtgccgtctctgcacgccgaggtgctcaatccgaacatccatttcgagagcacgcccttctacgtccagcgaacgctcgacgcgtggcgccagcccgaggtgcgcgagggcggggtgaccgaggtccacccgcgccgcgcgggcatcagctccttcggcgccggtgggaccaacgtccacatggtcgtcgaggagtatcaggcttcgactcctgccctcgagatcgcggcggccgagcctgagcttgtcgtgctctccgcgcacaccgaagagcggctccgcgctcacgccgagcggctgctgcgcttcttgcaaggctcgcggcctggagggctcccctcgcccagcgcgccgggccggcgcctgccggaggccgcgcagctccgcgccgagctgcgggacatcgtggcgcgacgcctggacgtcgcgccgcgcgacgtcgacgaggacgccgagatctgcgagctcgggctcggcgcgctcgacgtgcgccgcctgaccgaagacatcgagcgccgcttcggcctgcgggtgagccccgaggacgtgaccgagcggacgacggtcgcaggcctcgcagggcgcctgcgacacctggcagcgccggacgccgatcgggacgacagcgcggctcgtcccgccgtgcgcttgagcgatctcgcctataccctgcgcgccggtcgcgatccccgccagcaccgcctcgcgctgcacgtcgccgatctggacgagctcatcgagcagctccggcgctactgcgaggaaggcgcggccgacgggtcgcgctgcttcgccgggcaggcatccaggcgggccggaagcagcggatcgcgcaaggaggccatggcggacgaggcccgggtgcgcgccgccatcgcggagcgagacctggccacgctcggccggctctgggtcgccgggaccgacgtggactgggagccgctcgacgcgcgccgggcgcggcggcgcgtcccgctgcccacgtaccccttcgcccgcgagcgttactggttctccaggagcggagacgccttcaccctcggccaggcgggagagaggcgcttgcacccgctcgtgcaggcgaacacctccacgttccacgcgcacacgtactccagccggctccggggcgacgcgttctacctcgccgatcacctcgtgcacggccagaagctcctccccgcggcggcgttcctggagatggcccgcgccgccggggagatggcgtccgggcggccggtccgcgacatcctcgacgtcgtctggaccgcgcccgtcgtcgtgggcgccgagccgcgcgagatcgagataacgctccggccggccgccggcgccatcgacttcgccgtgtcctccgccgccgagcgcgcggtgatctcccacgcgcaggggcggatgcgcctcgacgagggggatcccgccgaagaggcggcgccgcccctcccgctcgatgacatcctctcacgttgctcgagggtcaccggcggagacgcgtgctaccgccgcctccagcagctcgggctgcaccacggcggcagcatgcgcgcgctccacgagctgcgccgaggcgagggcgaggccatcgcggagattcgcctcccggagcttcaccacgtggacttctccacctttgccctccatcccgccctgctcgacgctgccctgcaatgcacgctcgggctgctggacgatgaggcggcccgagccccctatcttcctttcgccgtcggccgggtcacgctgctccgcccgctgccggcgcggctcttcgcctatgccacgccgtcgtccgcgccgccgggcacgaacgccagggcctctcacgtcacgctggccgatcccgccggccgggtgctcctcgagatgcgtgatttcaccgtccgcctcgcgacggcggacgtcgcgcccacccccgcccagcggctctatttccggcctggcttgcgccctcagcgcgtcgaccgccccgccggcgcgcgcgccccgcaaggccccgtcctgctcctcgacaccgacgatgtcctctggacggccgccagggcgcgcctccaggcgccgatcggcctcgtccttccagggccggagttccaggcctcgagcgacgatcggtatgtcatcgatccgagccggccagagcaccatcgacgcctgctcgacgcgttcgtggcgcggcacggcgtgcctgcgtcggtcttgtatctccggtcgctgcatgacgaccgggaggccgccggcgacacccgccacctcgacgcggtgttgcacctctgccgcgcgctgcaggagcggcgaggcgagcgatccgttcgcgtgctctacgtccacccgaccgagggcggcgcggtcagcccgcgccacgcggcgctggctgccttcgcgcggagcgtgcgccgtgaggatcccaacctcctgtgcaggaccgtggccgtgccgctcgacgtcggcccaggccgcctcgccgacgcgttgctcgccgagtgcagcccggacgccgatcgcgcagatcccgccgccgaggtgcattaccacgagggtcagcggctcgtgcgctgcttcgagcccttccagcccgacgccagccggcccgtgccgctgcgggaggagggggtctatgtcatcaccggcggtgccggcgggctggggctcatcctctccgaccacctggcccggcggtaccgcgcgaagctcgtgctctgcggtcgctctccgctgtccgcgcagcaagcgtcgcgcgtccgcgccctcgaagcctcgggcgccgaggtcctggttctgcgcgccgacgtgagccagcgagaccaggcgtccgccgccctccacgaggcccggtctcggttcgggcgaatcgacggcgtcgtgcacgccgcaggcgccttgcgggacggcctgctgtccaagaaggacccggccgacgtcgacgccgtgatatccgccaaggtgacaggcacgctcctcctcgacgagctcacccgggaggatcatctcgacttcttcctgctgtgctcctcggtcgccgcgatcctcggcagcgccggtcaggccgactatgcctacggcaacgccttcatggatgccttcgccgccctccgcgaggagcaacggcacagcggccggcggcgcggggcgaccctctcggtcaactggccgctatggcaggaaggcacgatgaggcccgacgccgagtcgatcgcgtggatgacgcgggcgaccgggatggtgcccatggacaccgagcagggcctcgccgccctggaggactgcctgcgggccggagggccgcagatcgccgtgctcgccggcgatcccggcaagatccaggctctgttcagcggagagcgcgccgcgccggcggccggcggccccgccgcgctcccgcccgtcgagcccggcgcgtacgcgccccgcgcggtcggctttctcaagcgcgtgttctccgagcagtggcagctgccgatccaccgcatcgacgccgagcagtcgctcgaccagtacgggctcgactcgatcatggcgatgagcctcacccgccggctggagacgttcttcggcgagctcccgaagacgctgctcttcgagcaccagaccatcgccgcgctggctggctacctcgctcgccaccacgccgaggcgctccggcgcgtcgtcggcgacagcgccccggcggtcgctccgccgccccggcccgatgcggcccctcccggcgcggcgcccgcgcctcgcgagctctccgcctcgcgcctccctgcgccgcagcccgggggcctcgacatcgccatcgtcgggctcagcgggcgctaccccatggcgcctgacctcgacgcgttctgggagaacctcgcggccggccgcgactgcgtcgtggagatccccgccgaccgctgggaccacgggcgctacttcgatccgaacccgggcgcggcgggcaagagctacagcaaatggggcggcttcctcgacgacgtcgatcgcttcgatcccctcttcttcaacatcgcgcctcgggaggcggaggccatggacccacaggagcgcgtgttcctggaggtcgcgtggcacgcgctggaagacgcgggctacgcgcgatcgccgctggcgaaccgcgcgacaggcgtgttcgtgggcgtcatgtacggtcactatcagctcttcggcgccgaggcgctggcgctggatcggcccgtgtccgcgggctcgtccttcgcgtccatcgccaatcgggtgtcctatttcttcgacttccgcggccccagcgtcgcgctggacaccatgtgctcctcctcgctgaccgcgatccacctggcctgcgccgcccttcagcgaggcgagatcgagatggcgctcgccggcggcgtgaacctgtccctgcaccctcagaagtacatcctgctcagccgcggcaagttcatggccaccgacggccggtgccgcagcttcggcgagggcggcgacggctatgtccccggcgagggcgcgggggccgtcgtgctcaagcgcctggaccgcgcgatcgccgacggggatcgcatccatggagtcgtcaaggcgagcgccctcaaccacggcggcaagaccagcggctacaccgtcccgaaccccagcgctcaggccgacgtcgtcgccgccgcgctggcgcagtccggcgtcgatccgcgcacgatcacctatgtcgaggcgcacgggaccggcacctcgctgggcgatcccatcgagatcgccggactcacaagggccttcgaggcttccccgaaggagcgtcccacctgcgccatcgggtcggtcaagtcgaacgtggggcacctggagtcggccgcgggcgtcgctggcctcaccaaggtgctgctgcagatggcgcatgagcagctggtcccttcgatccacgcggatccccccaaccccaacatcaactttgccgagtcgccgttccgtgtacagcgggagctcggtccctggcgggctcctgtcgatgagcgcggccagcggctccccctgcgggcgggcctgagctccttcggcgccggcggcgccaacgcgcacctcgtgctggaggcctacgtgccgggcgacgaggcaggggccgcggccgccgtgacggccgggagcgagcgcccgcaggtgctcgtgctctcggcccgcacgcccgagcgcttgcgcgtctccgccgcgcggctgctcgatcacctccggacgcgcgcgcggggcacggcgctggccgatgtcgcgtacagcctgcaagtcgggcgcgaggccatggacgcgcggctggccctcgtggtcgacagcgcggagcaggccatcgcgctgctcgagcaccacctcggcgaccgcgcgcccgagggcggggcgccgcgcgcccaggagacgcaggggctggagcacatccacgaggggagcgccagggcgggccacgtccggcagctcgttcacggccgggcggccgcatctttcctgcaagccctcctcgatgaaggcgatctggacaggatcgccgcgctctgggtgagcgggtgcgacgtcgactgggcccgcctccacgagggagcgaggccgcgccgcgtcgctctgcccgcctatcccttcgcgcgcgagcgctgctggttcgccgtgcccgcagaggaccggcgcggcgggctcccgacctccgccgaggtcgcggcgacggcgcggctgcacccgctcctgagccgcaacacgtcgacgttcagagagcagcggttcgccacgaccttcaccggcgaggagatcctcctctcggaccaccggatccgaggccgcgccctgctgccgggcacggcttacctggagatggcgcgtgtggccggcgagctctccgccgagggccgcgtcggtcgtttcaccgaggtcacctggctgcagccgatccaggtcgatcgcggccccgtcgaggccaccctcgacctccggccgaccgagacgggctgccagtttcgcgtctgcacgcaggacggggccctcgtccacgtgcgcggccagctccacgtcgagccccagcccccgggaggcgagccgacggtggacctggcggccatcaaggcgcgctgccccgagcctctcctgcggcaggactgctatcgggccctgcgcgagcaagggttcgagtatggccctgcgttccaggtcatcgaggccttctacgacaacgacgaggaggccctggccctgctcagcgtcgccgagcctgatttccagggcttcgccggtgggctgcaccccatgatcctggacgcggccctccacgccgggatgctgcacaggcgagagggcgcgaccggcgacgtcacgccggtgcccttctacctggaagagctggtcgtccttcgcccgctggagcgccgctgctacgcgtatatgcaggtgcggcgcctcgccgcaggagaagagcggagcgaggtcgccgtcatggacgtgaccctcgtggacgaggcgggctcgccgctcgtgcgcgtcaaagggttcacggggcggaagctcgtcgacgccgacgaggagccggagcaaaacgccgtcctcttcttcggggacgcctggcagcccgccccgctcccctcgcgtccgcccgccggcgcgccgccggccagcgtcctcttgatcgccgaggacaccgcccgggcgcgggcgttcgagcgcctggtccgcgcgcggggcggtcacctgacgtgggtttgccctgtcgggtcgccccgggcgcaggccgagccgagcggcgcgccgagcgcggggtccggcgatcgcggggctccagggctcgcgatcgagccgcgccccgtcgacgactaccgcgggctgctcgcgacgttgaaggagcagggccgcctgcccggcgggatcatccgcctgtgggacgcgccgagcctcgacacggaagcgtcttcgcccgcggagggaccggagagcgtcgaggagctgagagagctcttccacctcgtcgtcgcgctcgcgagcgcggtccctcatccgaaggctcgcctgatcctcgccttccacggcgacccggcgcccctcgccgtcgaggccacgtccggcttctgcaggtccctcgggctgctgctgccgggcctgcggtcgagcacgatccactggacccaccgcgagcccgagcgccacgccgaggacctctgggccgagctcgccgatcctgcgacgagggggatcggcgggaggaacggggcggagatccgctatcgcggtccggaccggctcgcccgcaccgcggcgcccgccgcgctcgcgcccgacgccgcgccggccccgctccgccacggaggggtctacctcatcgcgggaggcgccggcgggctcgggtacctggtcgcccagcacctcgcccatcgctaccgcgcgagcctcgtgctcacgggccgctcgcccctcgacgccggcaaggagcggcagctcgccgggctccgggacgccggcggacaggggctctattgccaggcggacgtcgcggacgaggcggccatggcggccgcggtgcgcctggccaaggagcgattcggcgccttgcacggggtgatccacgcggccggcgtgctcgacgagcgccccgtcgtcgagaagacgtggggggagttccacgagaacctgcggcccaaggtcgccggcagcgcggtcctcgaccggatcaccgcggccgagccgctcgacttcttcgcggtgttctcctccacgtcggccgtgctcggagacttcggctcctgcgattacggaagcggcaaccggttccagatggcctatggcgcccaccgcgagcggctgcggcagcagggcctccggcgcgggatcaccgccgtcatgaactggccgctgtggcgcgagggcggcatgggcggtcgcgccgagtgggagcaaacctacctgaagacgagcggcctggattacctcgacacggccgccggtctggaggcgttcgagcgcatcctcggggcccgtcagtcgcccgtcacggtgttctacggcaagccgtcgcgtgtggcgagggccctcggcctcgacgcgccgccgcccccggcgggtcgcggcgcggcggccgcgccgctcccgccggcggaggcgccggccgccgccccggaggcggcggtccgcgagagcgcggcgcgcgcgccgctgcgcgaggtgatcctcgacgcgatcaccgaggtcctcaacgtccggcgcggcgcgatcgcgccggacgtcaacatcgccgagtacggcttcgactcggtgtcgcttgcgcagctcgccgatcagctcggcgcgcgcctcgggttgaagctggcgtcgctcgtgttcttcgagcacacgacggtggaagagatcgaggccttcctggagcggaagcacggcgccgagctccgcgcgcggatgaacggggcgcgggagctccacggccgcatgaacgaggcgcgagagctccatgaccgcatgaacggggcgcgagagctccacgaccgcatgaacgaggcgcgagagctccacgaccgcatgaacggggctcgaaaggaggctccgcgcgcgaaggagccggcgccggccgacccggctccgccgccggcgcctcgcgagaacggctcgcggctcgccggcgcgcctcgcgcgagcgcgccgcgcaggccgcaggaaggcgcctcgcgcggcgacatcgccatcatcggcgtcagcggccgctacccgcaggccgaggacctgcgcgcgctctgggcgcggctccaggccggcgagagctgcatcgaggagatccccgccgagcgctgggacaaggatcgctacttcgacccgcaaaagggccggagcgggaagagcgagagcaagtggggcggcttcctccgcgacgtcgatcagttcgatccgctgctcttcaacatccctcccgcgcgggctcggatcatggatcccatgcagcggctcttcctggagagcgtctatgagacgctcgaggacgccggctacacccgcgccatgctgtcgaaggacggcggcaaggtcggggtgtacgtgggcgccatctaccatcactacgccatgctcgccgcggacgagtcgacccgcagcctcctgctctcggccttcggcgcccacatcgccaaccacgtgtcgcacttcttcgatctccacgggccctgcatggcggtggacacgacctgcgcgtcgtcgctcaccgccatccacctcgcgtgcgagggcctgctcctcgggcgcacggatctcgccatcgccggcggcgtcaacctctccctcatcccggagaagtacctgggcctgagccagctccagttcatgagcggcggggcgctcagccgccccttcggcgacagcgacggcatgatccccggcgagggcgtcggcgccgtgctgctcaagccgctggatcgcgcggtccgcgatcgcgaccacatccacgcgatcatccggtccagcgccgtcagccacggcggcgccagcacgggcttcacggcgccgaacctcaaggcccagtcggacatgttcgtggaggcgatcgagagggcgggcatcgacccacgcacgatcagctacgtggaggcggccgccaacggcgctccgctcggcgaccccatcgaggtcaacgcgctgaccagggcgttccggcgcttcaccgcggacacgggcttctgcgcgctcggcaccgtcaagtcgaacatcggtcatctggaaggggcctccggcgtctcccagctcgccaaggtgctgctccagctccggcacggcgcgctggcgccgaccatcaacgccgagccgaggaatccgaacctgcacctcgacgacaccccgttctacctccaggagcgcctcgacgactggcgtcgaccgatcatctccggccgcgaggtcccgcgccgcgccatgatcaactccttcggggccggcgggggatatgccaccctcgtggtggaggagcaccgcccgccgccgcgcgacgccgcgccgggccgctcgccctccgggccgcccgagctgttcgtgctctccgcgaggagccgcaagagcctgcgcgagctggtcgtcaggatgcgcggcttcctcgccgaggcgaccgacctgcgcctcgacgacgtggcctacacgctccaggtggggcgcgaggccctggagctgcggctcgccgtggtggcggacaccgtggaggcgctcctctcggcgctggacggctacctccgcgatcccgaggtccccgcgccgggcgtcttcaccggccaggcggatggcgacgcgtccagcggcgccgccgcgcctcccgcccaggcgctccgcacgcccgaggaggcggcgcgccggtgggtcgcgggcgccgcgatcgactgggaggccctctaccccctccgcgacgcgcggcgcatcccgctgccgacctacccgttcgaccgccggcggtgctggctggatccggcgccctccgacgaggcctcgccgagccccgctgcgcccccgcccgaggcgccccggcccgccgcggccccgccggcgccccccagcgcggaggcccgcgcgctggagggctacctgtgcgcgcggctggagtccacgctgggcctcgatcagggcgagatctctgcccgcgcgtcgctgcggcgcctcggactggactcgatcctggccgccaagctcaaggtcacgctggagggagagctcgccatgaccatccccatggaggtcctgagcggcgacaagagcgtggcggagctcggcgattatctctctcgacggggagcccgcgcgccggagagccgggcgaaggcgcgcagcggcgcggccggggccgacctgtccacctccctcaaggccctctcgggcgcggtgctgcgggaacagttcctggcgttcgggcacgacctggccggcgtaccgggcgaggagctgactcggctctacgccatcctgcaagaggaatgatgacgatggaaagcgcgatgaccatccaggagtttgccaacttgtctgcggaggagaaggtgcaggtcctcctgcgcttgcgggaccggcgcgcttcgtggcaggcggcccccgagggccccgcggccagcgctcagccctcgctccggcccgtgatcacggcccgcccgggcgatcgcttcctccccttcccgctgacgccgatccaggagtccttcctggtggccaagcaggtcgacagggcgggcgatcacgtcggatgccacatctacctggagatcgacgaggcgcgcctcgacgtggcgcggctcgagcgcgccttccaccggctcgtcgtccaccacgacatgctccggaccgtcgttcgcgccgacggcacccagcaggtccaggagcccgggcagccgcgcagctttccggtggacgacctccgcgggcgcccgggcgcggcgctggacgcgcacctggagagcgtgcgcgcgagcatgtcccaccgggtctacgcgccaggggcctggccgctccacgagatccggatcacccgctgcagcgacgagcgcagcgtcatccacgtcagcatcgacgagtggatcctggacgccgccggcctcaacctcctgctcacccagtggtaccggctctacagcgaccctgacgcgaccctgcccgtctgcgacctcagcttccgcgattacgtcctggcctcgagggaattcgagcgctcgccggcctaccagggggatctcgcctactggtgcgagaagctggcccagatgcccgggggcccggcgctgcctcgcgccgagcagcccgggaggcccgcgggccgcgcctgctacccccgtcgccgcgtccacgggcgcctggccgaggcgccgtggcgcgcgctcaaggacaaagcgcgggagctggacgtctccccgacggccctgctcctcaccctcttcgccgaggccctcgcctcccacagcgcgcccgggccgttctccctcacgctcacgtacttcaaccgcccgccgatccacccgcacatcgagcgcctgctcggcccgctcatctccacccaccgcttcctcgtcgagggagccaccgatctcacgctgcaggaggaggtccagcgcagccagcgacagctctggcgcgacatggaccacgaccgcgccgacagcatcctcgcgctccgcgccctcagggcgaggcgcgcggcgccccccgcgagcacggtcgtcttcacaagcgtcctccacaacgtgagcagagaagcccggcagcaggggcggagcttcctcgatcaaatcacctattcggtcacccagaccccgcaggtctacctggaccaccaggtctacgagaaggacggcggccttcatttcacgtgggatgtcgtggacgccgtcttcgcgcccgggtgcgtcgacgccctcttcgacacgtattcgcggctcctcggggcgctcgcggcagagccctcgcgctggacgtcgccggggtggcgcgaggagctcctgggcccgcgccccccgcgcggcggcgggcccgaccggacctccgcggcgccggccggcgagggtctcgagatcatcgctcggccggaggagcgtcaccagagattccccctgtccgatctgcagcaggcctacttcgtcggccgcaccgggttcgccgccaacgggggcgtgagctgccagatgtaccaggagatcgagctccgcgatccggacatcgtccgcctcgatcgggcgtggcagcgcgtcatcgacgcccacgagatgctgcgcgcggtcatccacgccgacggcacccagagcatccgcgccgaggtcccgcgctacgtcatcgaggtctcggacctccgcgcggcgtcgcccgaggcccgcgcggaggccctcgctcggacgcgggagaccatggtcgccagggtattccccctggatcagtggcccttcttcgagctgcggctctcgctcaccgagccgtcgagggccgtcctccacctgagcatggatctgctcctcgccgacgcgacgagcatccacctcgtcctgaagcagctcttcgccctgtacgagcggcccgacgggccgtgcgccgcgccgcggctctccttccgcgactaccagctcgcgctcaaggaccacgagcgcgccgcgggccacgccgtcggcgtcgcgtactggcgccggaggctcgcggacctccccggcggccccgagctcggcatgcgcctgcccgacggccggggcggccgcctgcggcgccggcagttcgacggcgtcctggagcggtggtcgcgcctccaggagggcgccgcggccctcggggtctcggccgaggccgtgctgctgggcgtctatttcgaggtcctggacggccgctccagccggcgccccttcaccgtggtcgtggcgcgctgggaccggccgccggtgcacccggagatcggcgccgtggtcggcgatttcaccgcggtgagctggatcgtctcgccgccgggcgagaccttcgccgagcgcgtccggcacctggagcgcacgctctccgaggatcgcgagcaccgcctggtcagcggctcccgggtgctgcagcagatggccatcaagtcccggaacaggcagttcctcacgttcccggtggtcttcaccggcctcgggcccagcctcaagggcgacctccccgacaccgtctctctcggataccgcatcacccagaccccccaggtctacctggacaacatcagcatggaggccgacgacgccctgcggctccactgggactcggtcgagggcgtcttccccgaggggctcatcgagtcgatgttcggcgcttactgccgcatcctcgaccggctggcccgcgatcacgccgcctggcacgagggccggctcgacgcgccgcgcgcccccgagggccccgcgcccctgcccgcgccggagggccgcgaccgcgcgcccggcgccgcccggcaccggacgaccctgcaccggctgatcgaggagcgcgcgagcctgtgccccgaccatgtcgccctgatcgccgagcgcgagcagctcacgtaccgggagctcaaccgccgggccaaccaggcggcgcgccgcctgaggcggctcggcgtcgggcccgacgtcctcgtcggcgtgctcgccgaccgatccatcgagatggtcgtcgccctcctggccatcctcaaggcgggcggggcgtacgtgccgatcgaccccacgtacccccgcgagcggatcgacttcatcgccgaggacgccggcctctcggtcctcctcctcgcggaggagcgccgccggctcccgtcgttccgcggcacccagctgtgcctctccaccgagcggcacctcctggacggcgaggcggagcacgacctcggccccaccgccgggccggatcacctcgcttacgtcatctacacctccgggtccaccggcaagcccaaggggtgcatgatccctcatgacgcgatctgcaaccggctgctctggatgcaggacgagtaccggctggcgccggacgatcgcgtcctgcagaagaccccttatacgttcgacgtctccgtgtgggagttcttcctgcccctcatcgccggcgcgaccctggtgatggccaggccggaggggcacaaggacgtcgcctacctggtccgggtcatggaggagcagcggatcaccacgtgccacttcgtgccctccatgctgaacttcttcctcaaggagccggcgctcccaacgcacctccgccaggtgttcacgagcggcgaggccctgtcctacgacgtcatggacacgttcctgcgccgctccccggccaggctccacaacctctacggcccgacggaggccgcggtggacgtcacctactggccgtgcgagcgccggcccgatcgcaaggtgccgatcggccgcgcgatctcgaacgtcgagatccacatcctcgacagcgcgctcaggcccgtgcccgcgggcgccgagggcgatctctacatcggcggcgtctgcctcgcccgcggctacctcaaccggcccgagctctcgcgcgagcggttcgtcccgagccccttcgaccccggcgcccgcctctacaacaccggggatcgcgcgcgcaccctggacgacgggaacatcgagtacctgggccggctcgacgcccaggtcaagctgcgcgggttccgcatcgagctcggggagatcgaggcggcgctgagcgcccacgaggccgtgcaggacgccgtggtcgccgtgcaggacgcgcacacggaggaccccaagctcgtcgcctacctggtcacgggcggccggcccttcccggcgcccggcgccctcaaggcctatctcaaggagcgcttgcccgactacatggttccgaaccgcttcgcgcccatcgcccagatcccggtgacggcccacggcaagctcgatcgcaaggccctgccctggccggtgccggctccctcggcccagccggagcccccgcccgccggcgcggccgcggcgcccccgggcgccgcccaggcccggcggccagcgggcgtctccagggaggccgccgaggaagagctccagcgcatcctcggcaaggcgctgcacctcacccgcctcgatcccggcgctgacctcttcgagctgggcgccacctcgctcaccatcgtgcaggcgtcacagcacatcgaggagcgcttcggcgtcgggctgccggtcgaggtcgtcctggccgagccgaccctcgacgccatcgcgcggcacgtcgccgagcggacggcggctggcgcgcccgagcccccggcccccgggcccgcgctggacgcgcctcccgcggcgcccgagcccccggccgccgccgcccccggcccgatcgatttcttctccagggaagatcgggagcgcttcaagcagcagcagctccacctgcggcacggcgtcgagggcctcccgaccgtggatctggccgacgctcccgcggccccgcgcctctaccgcgaccgcgggagccgccgcgactaccggcccgagcccgtctcgttcgacgacctctcgcgcctcctcgccgtcctccggcggtacccgagcggccagcagacccagctctgctatccctcggccggcggcacctacgccgtgcagacctatcttcacgtgaaggagggcgcggtcgagcgcctcccggccgggatctactactaccacccggatcgcaaccagctggtgctcatcaacgatcggcccgccatccgccgggtgcaccacttctattacaaccgcgagcacttcgaccgcgccgggttcgggctgttcttcatcgcccagaccgacgccatccagcccatctacggcgatcagagcctcaccttcgccgcgatcgaggcgggggcgatgatccaggcgctcatgagccatcaggcggaggcggacctgggcctgtgcgccatgggagggctcgacttcgacgccatccgcgccgacttcaagctcgggagcgggcaccggtacatcgtctgcatgctggggggccgcgtcgatcgcgaaggcggcgggcggcagggccgcgcgaggctcctcgagagcgcgggggcggacggctcgtacggggcggccgcggcggaggccgccgccccgcgccgcgagcgcgaggctcccgccggcgcgcgcgagatcgcggtcatcggcctcgccggccgctaccccggcgcggacacgccacgccagctgtggcgggcgctccggagcggccagagcgccgtgacccggccgcccgccgggcgcttcggcgcgagcgccccgcagggcgacgagccccgaggcggcggagcctccccggggtggggcggctacctggagcggctcgaccgcttcgacagcctcttcttcggcatctcgcccgccgaggcgaagctcatggatccccaggagcgcctgttcatcgaggtggcctgggagtgcctggaggacgccgggtacacccccgaggagctccgtcgcgccgccccccgggtgggcgtcttcgtcggcgccatgtggagcgactaccagagcgtggggctggaggcgtggcagcgggaccggcgcgcgaaggccgtggcgttccactcctccatcgccaaccggatctcgtatctcttcgatctccacgggccgagcgtggccatcgacacctcctgctcctcgggcctgacagcgctgcacctggcgagccggagcctccggctcggcgagtgcgacgtggcccttgtcggcggggtcaacctccttggtcacccgttccaccccgacctgctcgagggcctcaacctcacgtcccgcgacgacaagacgcgcgccttcggcgccgggggcagcggctgggtgcccggcgagggcgtcggcgccgtgctgctgcggcgcctgcccgaggccgaggagcgaggcgagcacatccgctgcgtcctcaagggcacggcgctcgcccacgccggcaaggcgccgcggtacggcatgccgagcacgcgcgcccaggcgggctcgatccgtgacgccctcgcggacggcggggtcgccgcgtcggagatcgattacgtcgagtgcgccgccaccggctccggcatcgcggacgcctccgaggtcgacgcgctcaagcaggcgttcgaggggcggagccctgacggcccgccgtgcctcctcgggtcggtcaagccgaacatcggccacctcgagtccgcctcggccttgtcccagctgaccaaggtcatcctccagctggagcacggcgagatcgccccgacgctgcacacggagccgcgcaacccgctgatccagctcgacggcacgcccttccggatcaaccgcgcgctgtccccctggccccgggccgccggggcggacgcgcccccgcggcgggcgctcatcaatgcgttcggcgccaccggatcgtccgcccacgccgtcgtggaagagtaccggcctcgccgccgggcctcgacccccgcggcggccgtccccggcctgttcgtcttcgtcctgtccgcggacaccgccgagcagctcgaggcctgcgcccgcgcgctggcggagcacctgcgcgagcgctcgaccgcgcgtccgcgcgacgtcgcgccgccggccgcggccgcagacgtcgcgtacaccctccaggtgggccgtcgcgcgatggacgagcgcctcgccatcctcgccggcgacctggacgagctcgaggcccgcctgcgaggcttcctcggcgggcgtggcgaggacgacggcgagcacctcttccggggtcgcgcctcgtcgccgcgcgatcgagcgcccctgtccccggaggcgccgctccccgcgctggcgcgggcctgggtgaacggagcatccatcgcctggcacgacctgtacaccgacggatcgcggcgccgggtgcctctccccacctatcccttcgcccgcccgtcccactggctcggtcggcccgccggagacgccgcggcgcctgccgtcgcgcgcggcgagaccgccgaggaggcgccctcgcgcggcgagaccgccgaggaggcgccctcgcgcggcgagaccgccgaggaggcgccctcgcgcgagaccgccgaggaggcgcccgccgccctggcgccggcgaccgcggatcccgcgctgcgcaaggccaccctcggcctgctgtcctcctgcttcgccgaggtcgccgagatcccgcgccgcagcctcgatcccgaggtccccctggaccgctatggcctcaactcgatgctgatcgcccagctctccgcgcgactcgaggcgctcctcggcgagctgccgaagaccctcctcttcgagcaccacaccctggcagccctcaccgactggctggtcgcccaccgcggcgacgcgctcctccgccgcctcgacctcccgcggcgggccgcggggcccgcggcgtcccccggcgcgctccccgcggcgcccgcagcccgccgcgggccggcgagagagcgctcggccgcggcctctccggccctcgcgccggccgcgcctctcgagagcgtcgacatcgccatcgtcggcctgagcggccgctatcccggggccgacaccatcgacgccttctggagcaacctgcgacaggggcgtgacagcgtcaccgaggtgccggccgatcgctgggacgccgccgcgatcttcgaccccgagggaggccccggcaagacccgccagcgctggggtggcttcctcgatcgcgtcgatcgcttcgacgcgctcctcttcaacatctcaccgcgcgaggcggcgggcatggatccccaggagaggctgttcctggagatcgcctggtgcgccttcgaggacgcggtctatacccgcgagcggctcgccgaagaacaggcgcgcgccggggtgggtgccggcgtgttcgtcggcagcatgtaccagcagtactccatgctcgcccggacgcccgacgccggggcctcgtcgtccttctggtcgatcgccaaccgggtctcctacttcttcgatctgcgcgggccgagcctcgccgtggacaccgcgtgcgcctcgtccctcaccgcgctccacctggcctgcgagagcctgcgccggggggagtgctgcctcgcgctggctggcggcgtcaacctccacctccacccgcacaagtacgtcgccctcgatcgcctgggcctgctcgggagcggcgccgccagcaagagcctcggcgacggggacggctacgtgcccggcgaggcggtcggcgccgtcgtcctcaagccgctcgatcgcgccgtcgcggacaacgaccgcatctatggcgtcatcaaggggagcttcgccaaccacgccggcaagaccgccgggtacggtgttcccagccccgccgcccaggccgacctgatcgcggcggccctgcgccggacgggcatcgatcccgagaccatcggttatatcgaggtcgccgccaacggctcctccctgggcgacgcgatcgagctcgcgggcctcacgcaggcgttccgccggttcaccgcccggaagcacttctgcgccgtgggctcggtcaagtccaacatcggccatccggaggccgcgtcgggtatcgctcagctcaccaaggtgctcggccagctccatcaccggacgctggtgcccacgctccacgcggagccgcacaacccgaacatcgacctgagggacagcccgttctatgtccagcgagagctcggcccgtggacggcgccgaccctcgccggcgagggggggaccgcggagctcccgcgccgcgccgcgatcagctcgttcggggcgggcggcgccaacacccatctcctcgtcgaggagtactcgccccgcccggacgaccggggggacgagggcgcggtccccggcgcggtcatcgtcccgctgtccgcccggaccgcggggcagctgcgcgcgtacgccgcgacgctggcggacgacctggagcgccgctcgcgcccgcgcggccacggcgagcgggcgctcgccgatcgcgacctgaccgccgtggcatataccctccaggtcgggcgagaggccatgaacgagcgctcggccatcgtgaccgcgagcctcggcgatctcatcacgaagctgaggcagctcgccgcgggccagacggacgtcgacgatctccatgtgggcagcgccgcggcgtcgctctccgccctgatgctcgacggccgcgagggccaggcgttcctctcgatcctcgtggaggacggtcgccacgacaagctggcccggctctgggtgagcggcgcccggatcgactggcggacgctttacggcggctcgacgccgaggcccctgtcgctgccccactacccctttgccggcgaccgccactggctcgacgacgaggcgctgccgcatggcgccgcctggagcgcgaccgcggcgcctccggcccagaccgccgcctggagcgcgaccgcggcgcctccggcccgcgccgcggatcctgggggtgcggcgccgcccgaggggccaggcggcgcgcctccgggcggcgcggcccggcagcgcatcgcgcaggagctcacggcgatggtctgcgatgtcctcaagatgcaggccagggacgtcgacggggacgaggcgctccgcaactacggcatggattcccgcctctccgccgccttcatgcggtcggtgcagcagcggtacgggtcgagcgtgccgctcagcgccgcgcacacccatcccaccttgaaccagctcacggcccacattcatggcctcctgagcagcaacggcgcagcccggcacccgtccgccgcgcccctcgccgcgacctcgccgtcgatcgccacggccccggcggcctccgcagccccggcggcctccgcggccccggcggcctccgcagcccccgcggcctccgcagccccggcggcctccgcggcccccgcggcctccgcggcctccgcagtcccggcggcgctccacgaggctccggcgcctgatccgcgcgcgggggacgcacggcccggggcggacagcatcgccccgcagcccgagccggggcccaaccccgacgagctcgtcgtcatcaacccgcgcggctcacgcgggagctcgttctgggtgcacggcgcgcctgggctcgcgcagccgctctatcccctgtctgccgcgctcggcacggattacccgttcttcgccttccaggcccggggcgtcgacgggctcgccatgcccttcacgagcatcgaggagatcgcggcccattacgtcgcctgcctgcggcagcgtagtccgagagggccttacgtcgtgggtgggctgtcctccggcggcatcatcgccttcgagatggcccggcagctcctctcgcaaggggagcgcgtctcccggctggtcatgctcgacacctatcccgcggtcgcgggcctcgcgcaggagacgccgggcgacatcgacccgatcctgccgctcctgctcatggccaactccttcatcagcttcgatcgcgacggagacacggcgatcaagcccgacgacctcgccgggctcccccccccgatgcagctcccgcgggcggtgcagctgatcaaggagcggagccgcaccgcgctcagccgtgatcagatctacaggatgctgaacgggaacatcgctgtctacaagcacctcgacctcgcgtgcaggaagtaccagcccggggtcctcgacgccgtggacgtcctgttcttcaaagcggagaaaggcttcttcggcggagcgaacccgctggggctgcccatcctggacgtgttttcctcctatgactatgtgaccccgtggcgccagtggatacgcggaggcctgcaggtcgtggagctgccttgcgcgcacgtcgacctcctggagcccccggcgctccaccaggtggtcgcgcacgtccgcgaggcgctttcatgacaggtgagcggcgcgcgggcgccgagcccgcgggcgccgagcccgcgggcgccgagcccgcgcgccgcattgcgtttgatatcgagcgatccgcatgatagacgaccccgcgctgaaccctacgtggtctcgaccgctgagccagcgattccggggatcaagcgctctcccggtggcagctcgcgcgtgtcgttgctggagcgccgagccagaccggaccgagccaggcagccagggagagcgcagcgctgcgcgacgaggtgccctccttgcacagggcgacgaggagcgacgacgcgatgcgcccgccctcggctgcgcggcgacgggaggtcttgagagaggccctctcgggcccgatgacagacaatcagccgacaaggctctcaacggacggaaatttacatgacatcgatggcgcgacacctggacatccacgaggagctcccccagaccgctccgctgccgccacgcgcgatccagtggcgcaaggcgtttcggctggccaaggagcttacggagaagcccttcaccgccgagctctcctacgagctcatcttgtcgctcgacggcggggcgaccgagcgcatgtttcaagacttcctcgccgagccgggggcgcgcgcgctgatccagaagcggcccgacctggccgcgacgctgtccgacctggatctcctcggatccatgccagagggcagcttgggccgcacctacaaggagatgacggagcgggacgggtacgctgtcaacgggatcatccatgtgatgaaggcggtcccgaccttccaggaggtggcgccggatccccttcgccagtggttcagcttccgcggcgcggtgctccacgacgtcgcccatgcgctcacggggtacgggcgtgacctcgcgggcgaggtcgcgctcggcctctacctcgcggcggtttacccgccgtaccggagcggggtcgtgtattcgttcatcaccgcgctcgcgtcggtcacggcgccgcaggaccagaagctccgcaacctatcctacctgcgcgacgtgtggatccgcggccgccgctcgcgcatccccctcagcgcgccctgggaggacctgctcccgctccaggtggaggaggtgtgccgtatgtaccaggtcccgctcgtgcgcgagacgcacgcggagggcatcctccgcgatgcgttcgagaaaggtccctggataccgtcgttcaaggcgcagagctgggcatagccggcgcgcgcgccgaggcgagcccctggcgggcacgtcgtggcggcgcgcctcctccccgcggcgcgacgggctccctcgcgccgcggggaggaggcgcgcccgctcttctgcatgacccctgtgcaagaaccctgaggcggcctgggggccgaggaagaaccgatgaaagcatacatgtttcccgggcaagggtctcaggcgaaggggatgggacgggcgctgttcgacgccttccccgcgctcacggccagagcggatggggtccttggctactccatccgggcgctgtgccaggacgatcctgatcagcgcttgagccagacccagttcacccagccggccctctacgtggtcaacgccttgtcgtacctgaagaggcgcgaggaggaggctccccccgatttcctggccggccacagcctgggcgagttcagcgccctgttcgccgcgggggtgttcgacttcgagaccggcctcgcgctggtgaagaagcgaggagagctgatgggcgatgcccgcggcggcgggatggccgcggtcatcggtctggacgaggagcgggttcgcgagctcctcgaccagaacggcgccacggcggtcgacatcgccaacctcaacagcccatctcaggtggtgatctcgggggcgaaggacgagatcgcccgcctgcaggtccccttcgaggcggcaggggcgaagaagtacacagtcctgcgcgtgagcgccgctttccattcccgcttcatgcgaccggcgatggtcgagttcgggcggttcctggagggctatgatttcgcgcctccgaagatcccggtgatctccaacgtgaccgcccggccctgcaaggccgatggcatccgcgcggccttgagcgagcagatcgccagtccggtccggtggtgcgagtcgatacgttacctgatgggcaggggcgtcgaggagttcgtggagtgcggccacggcatcgtcctgaccggcctgtacgcccagatccgtcgagacgcccagcccctcgtcgtcgacgagggcgcggccgggctcgaccggcggggtccgccggcggagggccggtcgccggctgccttcggctcatcgaggctggcggcgcccgcgcagaacggggcggcggcgcccgcgcagaacggggcggcggcgcccgcgccggcggcgcatgcggccgcggcgcatgcgcccgcgcagaacggggcggcggcgcccgcgcagaacggggcagcggcgcccgcgccggcggcgcgtgcggccgcggcgcatgcggcggcgccgaacggggcggcgtcgccggagccggcggcgcccgcgccgaggggggccaggcggatctcgctcgaggtgctgggcagcgccgcgttccgggaggactaccgcttgcgctacgcgtatgtcgcgggctcgctggtcgatgggatctcctccaaggagatgatcgtgcgcatgggcaaggcgggcctgatcggctatctcgggaccaagggggtggcgctggacgccgtcgaggcgtcgatcctccacatccagcgcgagctccgcggtggtgagagctacggggtgagcctgtggtgcgacatggacgactcgcacctcgaatggcagagcgtcgcgctctacctcaagcacgatattcggtacgtcgaggcggtcgcctacatgcagataacgccggcccttgtctgctatcgtctcaagggcgctcaccgcgatcaccgcggcagggcagccacgcctcggcgcgtgctcgccagggtctcgaacctcgaggtcgcccgggcgttcatgagccccgctgcggatcacgtcctcgatcagctcgtgaaggacgggcggctcacgcgcgaggagggcgcgctcggccgggagctccccatcagcgacgacctgtgcgcgcacgccgactccggcggccccacggacatggggacggcagcggtgctgatgccggccatggcgcggctgcgcgacgacatgatgacgcggtacgggtacgaaaagcggatccgcgtcggcatggccggcggcctcggcgccccggaggcggtcgcgtccgcgttcatgctgggggccgacttcatcgtcaccaactccgtgaaccagtgctcgccggaggcgagcaccagcgaccgggtcaaggacatgctgcaggccgcgagcgtccacgacaccgcgtatgcgcccgccggcgacctgttcgagatgggagcccgggtccaggtcctcaagcgtggcgtgctcttccccgcgcgggccaaccgcctgtacgagctctaccggcactacccgtccctggacgcgctcgacgcgaggaccagggatcagctcgagaagcactatttcaggcgcgatctcgacgatgtctggcgggatgcgctgtctcgccggccggggacgcgcccggcggacgcggccaggacggagcgcgaccccaagcacaggatgtccctcgtcttccggtggtatttcgcccactgctcggagctggcgcggcgaggggacgaggagaatcgggtgaactaccaagtccactgcgggccggccatgggcgccttcaaccagtgggcgaagggcacggatctggaggactggcgcaaccgccatgtcgatgtgatcgccgagcgcctgatgcgggcgtccgccgatctcctcgatcaccgcatgcgcgcgctctcgcggtag

SEQ ID NO: 8 - protein, 6011 aa, Sorangium cellulosum
VEQDAIAIIGVACRFPGSPDYGRYWQLLERGEHAILEIPPGRWDPRAHYSPDFNKPGKSISKWCGLIDDIASFDHRFFNVSEREAKSMDPQQRLLLEEAWRCIEDSGVPLEQLRARKTSIYVGFMATDYHQESAAPGRPVDSYAALGSYGSILANRVSYTLGLRGASIAIDAACASSLVALHEARRALQRGESEFALAAGVSLNFHPWKYVSFSKSRMLSPDGLCKTFDADANGYVPGDGVGVLLLHPLAKAIAAGCHVYGVVAGSAVNHTGTARSITAPRVAAQRDVILEAYEDAGWNPETVTYVEAHGTGTSLGDPIEVEALTQAFRRYTTARQRCAIGSVKSNIGHLEAAAGVAGVIKVLMMLKHRVIPRTLHVQTLNPLIRFEETPFVVATRAMEWRAEGGEPLRAGVSSFGFGGANAHVLISEHGGARREPRPRGELRGPRGAAPRGETAGAPAEDGPLARAEELPSQQEDAAADEREGTVFLLSARSASSLSRAVRRWEAFVDDPLVKAGLATSLRDICATLAAGRQSFEHRHGFYIDDERDLRRLLKEPPARLEKTRPPRWVTRFGALALGQGRPAVRLLGARRLLDPHLDRIRRCLEELGIEHQDLRTYRQDGDPGRQELPYAFLFAHAYVSALADLGFTPYATSGEGHGIWLALAQSGVLPLNEIVSVLSGAGELQRLSPRRPRLPLFDPIHSTYLMPYLLDAGYVRALVEGLAVPAATLRDLLARARLLLRAQFTFKKFLSEWSPALQALGTTPERLLEEELPASDARASLIALIAQSCVRKLNRRWQLTDAPSSGDPRFDELVDLVVDGLLPREALVQLALGDRADLHEIAGTLHRRQDLLDLSQPYGILRRRSERLDPSEIDDFSGWIRQIAGLEAPGLPPEEGVAFLELGRVARRAQRAPGPDLSVPALDSPLQLVALRLWLEGTDIRWGELFPEGSFAKIPLPGYAFDQAQFWLPAAREGTSPPEDARDDADARHAAVAPHGAADRAERPSIPVDRLIADHVIQGRAIVPGALMVEMALEASQRAHGRPAAVLRDIVFQRAVPLDAHANLTIDVDPDGGRFVGRDGAQGACRGAYGSAPPSPLEPLDAPARDGDRRRDDSLYRDLSRVGYRYGESLQVIAATGRVGSRHVFELRSSVARTTPVAGFDPALFDGLLQAALVVGQRLGLFGGGGAIYVPQAIALVERLAPVDGGCLVCIDERDLSIKEYGLTVDLRAYDPSGAGLLRVEGIFFRKVLPGFVESSPARVTGGAAEAPRRAGAAGDPESAAPRAACYQPVWERRPLPDRGGAPPRGRAVAIIRSEADSAAWLSPLRARYSQVTVARLGSPPGEAGEDRLVLGDDREEGFSELVRRAERAAAGEAVDIYLLDALTPDARVPSRAPAALEPALGPREEAAARSAFLLAKALVKSAAPWRLVIGTRRCQAVVPGDRGEGFRHGVLAGMARTLTQENPRVQVHLVDFDAAPPLACAGHLVEECGVLGPGDWVAYRDGARYVRAFAPVEEPGATATPPFQDGRVYLLVGGAGGLGLGLAGHIASRAHARLVLLGRSPLGHEAERRLARLRGDGGETLYISADVSDPQQCEQALAAVRQRFGAIHGVVQMAGVVEDKLIAGKTWESVRREMAPKVQGTWLLHELTRRDPLDFFVTFSSVVSLLGNHGQVGYAAANGFLDGFIHHRARTGAAGRSLGVNWTLWEDGGMGAAPGIVRRFSARGLPPIRQHDAFGALERLMTGGRSPQALVLAEPAEHLFARASTRPAPHAVAPDPERGDREQARDKEQVRGDASMTRTTANPHGTAPAGAGQDGRRIARIEEDLRRLVSARIEAPSQAVDAEESFFSLGVDSVALQEITETLERTYGSLPPTLLFENPNIRQLARYLAERVPARSAAPAEVEPAQAPASGPAEAPPAARAAVPLPAPEPPGEAASRGARVAAVAAGQEHDTPGAPSTRAARRESPSDGPAIAIIGMSARFPKSPDLDAFWQNLLSGRDCVDEIPAERWDHRRYFAEAAQPHKTYGRWGGFIEDVDRFDPMFFNISPREAEQMDPQQRLFLECAWATMEHAGYGDPRAYGDRAVGLFVGVMWNEYSRIGSQLTLQTARYAGPGSLYWAIANRVSYWMNLTGPSLAIDTACSSSLVAVHQACMSIRNGECDMAMAGGINLSIHPDKYLYLAQSKFLSLDGRCRSFGQGGTGYVPGEGVGAVLLKPLEQALRDGDHVYGIVRGSAINHGGRATGFTVPDPEAQARLVFDALRRARVSPDQLSYIECHGTGTALGDPVEIAGLSKAFRMAGATRTSIPIGSVKSNLGHLEAAAGIAALIKVLLCMQHQAIPKSLHSDVKNPNIRFEEVPFEVVNETRSWQGDGGAPRFAGVSSFGAGGSNAHVILESYEPHVRLSAGDDAAEGGALIVLSAKDRERLDALAGRLRDFLRERAGRAPSLSDIAYTLQLGRQHMDHRLAIVAASREDLLAKLDAVLAGRGEVPGAFRGDVHGDKAASLAMDGDDHDREYLERLARDRRLDRLARLWLLGLRVPWEELHRDRGRKRVALPTYPFARERYWLPDVESSITAAAPVEAPASEQAPAPRGEKGLPEDFFFHEQWSVAPLDPATGSDGAAVRSALVIYTPEGEALADALIARHPGARVARILLGAGQGAKGRPGPEARAARLPPAQEVQADDPAALERALRELAAAGVAGLDAIYFLGGLAAQEPAAGDLEAVERAQQRGLLSLFRLAKALGALGLSSSPCQLKIITNDACSVRTGDPERPLAAGLYGLARSIAKEYPRLNVSCIDIQTRALSHPADEGLISAVIAEPGHLRGREVALRDGKRFQRTMAALPLQPPARDPYRPGGVYLVLGGAGGLGHLFSQHLAGTYRARLVWIGRRPLEADIRSRIADVEARGGEVLYLQADAGDPSSLRAAVSRAKARFGAIHGVIHSAVILGSHPIATTDEATFAAGVRAKIAGSVALHQAVADEPLDFLLYFGSIASYLNNGGASPYAAGCTFQDRYAAFQRSRVPYPVKLINWGYWGDVGAVAGNTETHDQQFNAIGVGAIAPEDGMEAARRVLAQRLPQVIAAQLTRPPQSLFGYDLSHEATVHPERFEPLLERSVPRIQPGLSAVRELLTHQPAFDALERFSEDLLLCIFQDMGAFQRAGSAESAATLRERLGVAGRFGRLYDSLLAILEGAGYLRIEGDRLFTSERVTPKKHEVERRMQQLADLPAIAPYVRLLWACYRRYPELLRGQVAATDVLFPQGSMDLMGPLYKGNATADHFNELVIKSLLVFLDARVPHLREGEKITILEVGAGTGGTTASVLEALSSHARHLEYFYTDISHAFTRYGKRQYGPRYPFVTFQPLDLEGDVVAQGFSAERFDVVLGANVVHATKNLRSTLQSIKRLLKANGWLVLNEMTRVVHFLTLSAGLLDGWWLFEDAAERMKWSPLLSSPMWKGLLEEEGFRRVAPLQHSDGTSSWSIQNVILAESDGVSRSRRTESAAPRPAPSATSAAAASEALPPAPSTPAAEPVAAFRPMSLQAVEDKIIDSLASTLQIDRSKLSSDVPFTTFGVDSIFAVEVAGVIGRELSIDLRTTALFNYPTARALAEHIAATFAPSEAAPARAPEPAAQPREQLPSSPPQPAPGAPPRPAQATSQVQAPAPERPPAPQPAGAQQRVRQLALGALAEVMAIDVRELDPSATLAECGIDAQQAVVVVSRMNQALGTSATAMDLLRCGTLADFVDHLLASSPAPRPDAETRPGTAAALPAPAPPAAIEPRSARSTDIAVVGMSCRLPGAETVADFWRNLCEGHNAIREIPPDRWSLDGFYDPDPSVAARSYSKWGGFLDNIGDFDPLFFGISPLEAELTDPQQRLFLQEAWKAFEDAGYSAEALSGQRCCVFVGCKDGDYVYKLGPSADASYRLIGNTLSILAARISYFLNLKGPSVPVDTACSSSLMAIHLACQSLISGSSDLAVAGGVALMTTPVSHIMLSKTGMLSPTGSCRTFDDSADGLVPAEGVAAVILKPLDAALRDRNHIYGVIRGSEANQDGKSNGITAPSTPSQAALEVEVYRKFGVHPETIGYVETHGTGTKLGDPIEIHALTDAFAAFTDKKGFCPVGSVKTGIGHTLAASGAASLIKVLCCLQHRTLVPSLHYDRPNRHIHFENSPFYVNTARRHWAHAGDLPRRAAISSFGMSGTNVHLIVEEAPPEADATAPTVAPYTLIPISAKAPAPLHRRVADLAAWLDAGGRDRELGDIGYTLGVGRSHFPLRLAFVARDTRDLRRQLAAWLARHPTADDVPAPAARPEPALGQTAGRLASELRDAPPLTADAYREKLEALAHAYVAKHDPEWQSLFAGQDRRLISLPTYPFNNRRFWVDEPSRYGLDHAAPAASAAPAPRPEPAPAARLAAPAEQPGHGDRRADSLLYFRSAWETAEHEAAAGQLRAPILLFDDGGAVRERLLDSDRPVIAVTPGPGFRELGGGRYELNPGDAADYGRLVAACKQRGALPREVVYLWPLARAQAQAEPTAPFFQATSLCRALADHRPAHGEAVRILYVYWQDGDRLDASHAAMSGLARSLQLDLPHLHWKTLGLEPRTADGALCDLVLAELLAPPQGAVRYQRGHRQIQRLQPWRPEGEASAPFRSKGVYLITGGAGGLGGLFAEHLARRHQARLVLCGRSPLTPAGDDLLRRLAQLGAEAVYVRADVADREDVFALLGRVEARFGALHGVLHSAGVTADASLRNKSRDQMVAVLAPKVLGTLHLDDATRHRELDFFALFSSVTAVMGNMGQTDYGYANSFMDHFAAWREAERQSGRRSGRTVSINWPLWRDGGMSVSQEMQTLLTSTLGMSALSSDAGIQAFERAVASAHPQVVVLAGDEAKIQESLGIAAPTPPAGALPGSHGAPPAARAKAPPARSALAKQVEELLLQAVSGVLKVAREELNYDAPLRDYGLESINVIALTNHLNRTYALDLKPVRFFEHETLAALGGWLCEERGEHLARRLGPSRAPEAGLPAAPAAPPEPAQAAPAQPAKEPPARSARAAERVPPEAPSARAERGMAAHEPIAIIGIGGALPKSSDLSAFWQHLVDGRSLVSELPADRWDWRAYDNGDANRKGLRWGSFYEDMDKFDPMFFGLSPREAELMDPQHRVFLETVWKAIEDAGYRPSDLARSNTGVFVGASSLDYLELMNGHRTEAYALTGTPHSILANRISFLLNLHGPSEPINTACSSALIAVHRAAETLRSGACDLAIAGGVNAILSPATALAIAKAGMLSPDGKCKTFDRSANGYVRGEGAGALLLKPLRRALADGDHVYAILRGSAENHGGRANSLTAPNPRAQADLIIAAFRAAGVDPATVGYIETHGTGTALGDPIEINGLKTAFEQIYKDHGRPPPQAPHCGLGSVKTNVGHLEAAAGIPSLFKVLLAMKHRKLPGTLHLHDLNPYIELEGSPFYIVTRTEDWKPALDADGRPLPLRAGISSFGVGGSNAHLVLEEHHDERAEEPSAAEVRRGPHLIVLSAKSEERLHAYVDALIAYLRDTAPERRPSLGHIAYTLLTGRDVMDARLACVATDTDDLVTRLSRYRAGESAVDGLFTGRSDGSSSAAAVLIEGEEGQQFVEALLRNRKWAQIARLWVAGRTGIDWSSLFDGERVRRVPLPTYPFARERYWVPDEIGKEHAGNGAPPAVNGKAHNGAAEGGARPPASAGSTLRPTLDAARSSPERPVFQKELEADAFYLRDHVIAGNIILPGVGHLELARAAGELAGGRPVRVIRDVLWAKPILLDGPRLDVQVAVSHDRQGAEYQIRHEGEGREVLYSRGRLAYEPAPRRDGEPERRDVKAIRSRCHDRKDHDTFYRRYREAGFRYGPSFRVVQEAWGNERESLGALVLPDHLREGFPQFGLHPCLLDASLQSITGMQLDAGRDAPSMSIPFAMGQLEIFGPLPPVCYAHATLGSRRGEGAREIVKYNVAVLDEDGLVLARITDFSARAFTNDQPRSPAEQAAAPLGYYQSTWTRSAL

SEQ ID NO: 9 - protein, 6256 aa, Sorangium cellulosum
MSSTLRHTDTILVLLPASAAFSGLDERLVAQVSDPQRLVFVQAGERFASIDPRHYRVDPARPEDYVRLFSELERSGALPTHILHAGNCVGPSALGAGERDAFASIRERLGQELERGLYAILSLVQAKLAVNPAGPTRCVFAFTTDEAHPRPHHEAVGGLAKALTTVDHRFQLVTVQMDACDADTAARRLIEELTSPHHQNGGEVRYRGGERFVHEVQRLEPAPERGEPPAALPLRAGGVYLVTGGGGGLGMLFARHLAVKYGARLVLSGRAPLDDDKRAKLRELEALGGRAAYVPADVGDEAETRRLLSAVSAEFGELHGIFHCAGVADRTPLARATIADFERVLRPKVHGTLHLDLETRDRDLDVFVLFSSISALVGDFGAGSYSAANCFLDRFADAREQLRRSGLRRGQTLSVNWPLWQDGGMRMQEQDKAMYFQFSGMGALEAAEGIEAFEGALRAGRPQLLVVTGDRKKIDRILQVREPRSAAAPREEPQRPAAGGAAPPAASHPGSSEGRGASGGERSSSAPQGSPRAATRGPLPREQLLAQCRDYLRNLIAQATKLPVDKIHADRDLEDYGINSLMIMELNSMLDRDFDALPRTLFFEYKNVAELAAFFADEHGSRLQQILAGGTDSSPDATPPPEEQPPAPEPDAAAALAAAPAPAPRPPPAALRQDDGHIAVIGYGGRFPKADDPEAFWRILKEGIDCITEIPRERWDWRAYHDDVPGTPGKIYCKWGGFINDFDRFDPLFFRLSPRAAHSMDPQERLFLTVAWETLEHAGYTLDRLNRGSDGPPGGAGRRNRVGVFAGVMWSDYGKHGQDELHKGNPVIASADYSSIANRVSYALNLHGPSIASDTACSSSLVAIHLACESLRRGECHYAIAGGVSLSLHPAKYLQMSNLKALSAEGKCRSFGAGGAGYVPGEGAGALLLKPLRQAIADGDYIHAVIRGTAVNHDGKTNGYTVPNPNAQADVISQALRQAGVDARTISYVEAHGTGTELGDPIEVTGLSKSYRTDTKDRQFCALGSAKSNVGHLEGAAGVAGVIKVLLQMKHKQIAPSLHSRELNPSIDFASSPFKVPQELSAWERPRLARPDGAGEIPRRAGVSSFGAGGTNAHVILEEFENAPRATSGREDVLVVLSARSEERLRAYAGKLAASLQLRLAGEDAAEHLDLERIAYTLQTGREAMDSRLAIIASDPRQLIADLEAYSEGRLDDKGPRCFSGTVKPYELPELEATHQAAIDEAAASYDLRALARQWIAGYAIDWPRLYPSPPPYPLALPTYPFARDRYWIPVAAQAPAVAAAAAKGLHPFLDANVSTLEELAFEKTFARGDLVLRDHVIAGRPVLPAAVYLEIARAAGHHAGPGPVSGVQDATWARPIVATGDSVTLRVSLARERQSVIYRVTSQPEGQPVVHGSGHLTFAAPAAAPPPASLRDIIARCPRQISADDLYRSFEALGIHYGPAFRPVQALHCGEREAVAVLRMPDAAGSGDYALNPSLLDGALQAIVHIGLDNELDPSLLRLPFALGRLVIRRPLDATSCHAHAILTHESRAGEDRVLKYRIDVYDGDGALLVEIVDYSVRVVARDALGPAGARASQPAHTLWYEPRWEATPAAPGRASAAWDRLPERLLVFGRDDELTSRLVEALSRLRPTRRIVPGAAFGALDRQGYRIDPADPSHYRRLSEELDRDDPWSTRTVGVIHLWRYPAGAEGAHAGLHSLLYLVQSLTARNAAQRVRCLVAVGSTDGAADPRDEALAGFGAALSPVNPHLELITLQADATRLDAQQMAGVLLHELAASDTAHGSEIRYTDAAARWTRALRPLEDGPTRTADAPPLRTGGVYVITGGSGYLGSTFARHLAGRYGARLVLCGRSSNDERKEALVRELRGLGGEAVYVQADVSDAGAAQRVVQAAQQRFGALHGILHAAGTDEAPPLARADAASFAKVLDPKVRGTLNLDAASRQVVTLDFFALFSSIAAVMGDLGAGCYAYANAFMDRFAAARERQRAQGRRHGKTLAINWPLWAGEGMSLPEGQQELYAGIAGMRALDPALGLELFARALSAPAPQLLVVHGDPERMRRVIERRNPRPAAASSHPAEPAASAAPGDERLAQAVEDYLKGHFAAVFKMDAAQIDPQTSFDDYGIDSLVIVELHARLDKDMTPLPRTTFFELRTVRAVADHLLASRRAELRRVVGLDREATAPPAPEAGEPARRGGAEAPAHAVAPGPAASASSNEHAGAGAGRDAGSRAPARPGAALADEGIAIIGMSGRYPMAPDLDAFWANLKAGRDCVEEIPAERWDHRRYFDPEPGKEGKSYCAWGGFIEDVDKFDPLFFQISPKQVATMDPQERLFLETAWATLEHGGYGRVQEDAARIGVFAGVMWDDYGLLGLEQAALGNHVPAGSDHASIANRISFVMNLRGPSLTVSTACSSSLLAVHLAVESLRRGECAMAIAGGVNLSIHPSKYTRLCQLQMLAPDGRCRSFGAGGKGYVPGEGVGAVLLKPLSRAEADGDTIYAVIKGSAVNHGGKTHGYTVPSPKAQADVIGRALERAGVHARTISYVEAHGTGTALGDPIEVGGLEESFRRDTGDRQYCALGSVKSNIGHLESAAGIAALTKVALQLHHRQIVPSLHAEVLNPNIHFESTPFYVQRTLDAWRQPEVREGGVTEVHPRRAGISSFGAGGTNVHMVVEEYQASTPALEIAAAEPELVVLSAHTEERLRAHAERLLRFLQGSRPGGLPSPSAPGRRLPEAAQLRAELRDIVARRLDVAPRDVDEDAEICELGLGALDVRRLTEDIERRFGLRVSPEDVTERTTVAGLAGRLRHLAAPDADRDDSAARPAVRLSDLAYTLRAGRDPRQHRLALHVADLDELIEQLRRYCEEGAADGSRCFAGQASRRAGSSGSRKEAMADEARVRAAIAERDLATLGRLWVAGTDVDWEPLDARRARRRVPLPTYPFARERYWFSRSGDAFTLGQAGERRLHPLVQANTSTFHAHTYSSRLRGDAFYLADHLVHGQKLLPAAAFLEMARAAGEMASGRPVRDILDVVWTAPVVVGAEPREIEITLRPAAGAIDFAVSSAAERAVISHAQGRMRLDEGDPAEEAAPPLPLDDILSRCSRVTGGDACYRRLQQLGLHHGGSMRALHELRRGEGEAIAEIRLPELHHVDFSTFALHPALLDAALQCTLGLLDDEAARAPYLPFAVGRVTLLRPLPARLFAYATPSSAPPGTNARASHVTLADPAGRVLLEMRDFTVRLATADVAPTPAQRLYFRPGLRPQRVDRPAGARAPQGPVLLLDTDDVLWTAARARLQAPIGLVLPGPEFQASSDDRYVIDPSRPEHHRRLLDAFVARHGVPASVLYLRSLHDDREAAGDTRHLDAVLHLCRALQERRGERSVRVLYVHPTEGGAVSPRHAALAAFARSVRREDPNLLCRTVAVPLDVGPGRLADALLAECSPDADRADPAAEVHYHEGQRLVRCFEPFQPDASRPVPLREEGVYVITGGAGGLGLILSDHLARRYRAKLVLCGRSPLSAQQASRVRALEASGAEVLVLRADVSQRDQASAALHEARSRFGRIDGVVHAAGALRDGLLSKKDPADVDAVISAKVTGTLLLDELTREDHLDFFLLCSSVAAILGSAGQADYAYGNAFMDAFAALREEQRHSGRRRGATLSVNWPLWQEGTMRPDAESIAWMTRATGMVPMDTEQGLAALEDCLRAGGPQIAVLAGDPGKIQALFSGERAAPAAGGPAALPPVEPGAYAPRAVGFLKRVFSEQWQLPIHRIDAEQSLDQYGLDSIMAMSLTRRLETFFGELPKTLLFEHQTIAALAGYLARHHAEALRRVVGDSAPAVAPPPRPDAAPPGAAPAPRELSASRLPAPQPGGLDIAIVGLSGRYPMAPDLDAFWENLAAGRDCVVEIPADRWDHGRYFDPNPGAAGKSYSKWGGFLDDVDRFDPLFFNIAPREAEAMDPQERVFLEVAWHALEDAGYARSPLANRATGVFVGVMYGHYQLFGAEALALDRPVSAGSSFASIANRVSYFFDFRGPSVALDTMCSSSLTAIHLACAALQRGEIEMALAGGVNLSLHPQKYILLSRGKFMATDGRCRSFGEGGDGYVPGEGAGAVVLKRLDRAIADGDRIHGVVKASALNHGGKTSGYTVPNPSAQADVVAAALAQSGVDPRTITYVEAHGTGTSLGDPIEIAGLTRAFEASPKERPTCAIGSVKSNVGHLESAAGVAGLTKVLLQMAHEQLVPSIHADPPNPNINFAESPFRVQRELGPWRAPVDERGQRLPLRAGLSSFGAGGANAHLVLEAYVPGDEAGAAAAVTAGSERPQVLVLSARTPERLRVSAARLLDHLRTRARGTALADVAYSLQVGREAMDARLALVVDSAEQAIALLEHHLGDRAPEGGAPRAQETQGLEHIHEGSARAGHVRQLVHGRAAASFLQALLDEGDLDRIAALWVSGCDVDWARLHEGARPRRVALPAYPFARERCWFAVPAEDRRGGLPTSAEVAATARLHPLLSRNTSTFREQRFATTFTGEEILLSDHRIRGRALLPGTAYLEMARVAGELSAEGRVGRFTEVTWLQPIQVDRGPVEATLDLRPTETGCQFRVCTQDGALVHVRGQLHVEPQPPGGEPTVDLAAIKARCPEPLLRQDCYRALREQGFEYGPAFQVIEAFYDNDEEALALLSVAEPDFQGFAGGLHPMILDAALHAGMLHRREGATGDVTPVPFYLEELVVLRPLERRCYAYMQVRRLAAGEERSEVAVMDVTLVDEAGSPLVRVKGFTGRKLVDADEEPEQNAVLFFGDAWQPAPLPSRPPAGAPPASVLLIAEDTARARAFERLVRARGGHLTWVCPVGSPRAQAEPSGAPSAGSGDRGAPGLAIEPRPVDDYRGLLATLKEQGRLPGGIIRLWDAPSLDTEASSPAEGPESVEELRELFHLVVALASAVPHPKARLILAFHGDPAPLAVEATSGFCRSLGLLLPGLRSSTIHWTHREPERHAEDLWAELADPATRGIGGRNGAEIRYRGPDRLARTAAPAALAPDAAPAPLRHGGVYLIAGGAGGLGYLVAQHLAHRYRASLVLTGRSPLDAGKERQLAGLRDAGGQGLYCQADVADEAAMAAAVRLAKERFGALHGVIHAAGVLDERPVVEKTWGEFHENLRPKVAGSAVLDRITAAEPLDFFAVFSSTSAVLGDFGSCDYGSGNRFQMAYGAHRERLRQQGLRRGITAVMNWPLWREGGMGGRAEWEQTYLKTSGLDYLDTAAGLEAFERILGARQSPVTVFYGKPSRVARALGLDAPPPPAGRGAAAAPLPPAEAPAAAPEAAVRESAARAPLREVILDAITEVLNVRRGAIAPDVNIAEYGFDSVSLAQLADQLGARLGLKLASLVFFEHTTVEEIEAFLERKHGAELRARMNGARELHGRMNEARELHDRMNGARELHDRMNEARELHDRMNGARKEAPRAKEPAPADPAPPPAPRENGSRLAGAPRASAPRRPQEGASRGDIAIIGVSGRYPQAEDLRALWARLQAGESCIEEIPAERWDKDRYFDPQKGRSGKSESKWGGFLRDVDQFDPLLFNIPPARARIMDPMQRLFLESVYETLEDAGYTRAMLSKDGGKVGVYVGAIYHHYAMLAADESTRSLLLSAFGAHIANHVSHFFDLHGPCMAVDTTCASSLTAIHLACEGLLLGRTDLAIAGGVNLSLIPEKYLGLSQLQFMSGGALSRPFGDSDGMIPGEGVGAVLLKPLDRAVRDRDHIHAIIRSSAVSHGGASTGFTAPNLKAQSDMFVEAIERAGIDPRTISYVEAAANGAPLGDPIEVNALTRAFRRFTADTGFCALGTVKSNIGHLEGASGVSQLAKVLLQLRHGALAPTINAEPRNPNLHLDDTPFYLQERLDDWRRPIISGREVPRRAMINSFGAGGGYATLVVEEHRPPPRDAAPGRSPSGPPELFVLSARSRKSLRELVVRMRGFLAEATDLRLDDVAYTLQVGREALELRLAVVADTVEALLSALDGYLRDPEVPAPGVFTGQADGDASSGAAAPPAQALRTPEEAARRWVAGAAIDWEALYPLRDARRIPLPTYPFDRRRCWLDPAPSDEASPSPAAPPPEAPRPAAAPPAPPSAEARALEGYLCARLESTLGLDQGEISARASLRRLGLDSILAAKLKVTLEGELAMTIPMEVLSGDKSVAELGDYLSRRGARAPESRAKARSGAAGADLSTSLKALSGAVLREQFLAFGHDLAGVPGEELTRLYAILQEE

SEQ ID NO: 10 - protein, 3792 aa, Sorangium cellulosum
MESAMTIQEFANLSAEEKVQVLLRLRDRRASWQAAPEGPAASAQPSLRPVITARPGDRFLPFPLTPIQESFLVAKQVDRAGDHVGCHIYLEIDEARLDVARLERAFHRLVVHHDMLRTVVRADGTQQVQEPGQPRSFPVDDLRGRPGAALDAHLESVRASMSHRVYAPGAWPLHEIRITRCSDERSVIHVSIDEWILDAAGLNLLLTQWYRLYSDPDATLPVCDLSFRDYVLASREFERSPAYQGDLAYWCEKLAQMPGGPALPRAEQPGRPAGRACYPRRRVHGRLAEAPWRALKDKARELDVSPTALLLTLFAEALASHSAPGPFSLTLTYFNRPPIHPHIERLLGPLISTHRFLVEGATDLTLQEEVQRSQRQLWRDMDHDRADSILALRALRARRAAPPASTVVFTSVLHNVSREARQQGRSFLDQITYSVTQTPQVYLDHQVYEKDGGLHFTWDVVDAVFAPGCVDALFDTYSRLLGALAAEPSRWTSPGWREELLGPRPPRGGGPDRTSAAPAGEGLEIIARPEERHQRFPLSDLQQAYFVGRTGFAANGGVSCQMYQEIELRDPDIVRLDRAWQRVIDAHEMLRAVIHADGTQSIRAEVPRYVIEVSDLRAASPEARAEALARTRETMVARVFPLDQWPFFELRLSLTEPSRAVLHLSMDLLLADATSIHLVLKQLFALYERPDGPCAAPRLSFRDYQLALKDHERAAGHAVGVAYWRRRLADLPGGPELGMRLPDGRGGRLRRRQFDGVLERWSRLQEGAAALGVSAEAVLLGVYFEVLDGRSSRRPFTVVVARWDRPPVHPEIGAVVGDFTAVSWIVSPPGETFAERVRHLERTLSEDREHRLVSGSRVLQQMAIKSRNRQFLTFPVVFTGLGPSLKGDLPDTVSLGYRITQTPQVYLDNISMEADDALRLHWDSVEGVFPEGLIESMFGAYCRILDRLARDHAAWHEGRLDAPRAPEGPAPLPAPEGRDRAPGAARHRTTLHRLIEERASLCPDHVALIAEREQLTYRELNRRANQAARRLRRLGVGPDVLVGVLADRSIEMVVALLAILKAGGAYVPIDPTYPRERIDFIAEDAGLSVLLLAEERRRLPSFRGTQLCLSTERHLLDGEAEHDLGPTAGPDHLAYVIYTSGSTGKPKGCMIPHDAICNRLLWMQDEYRLAPDDRVLQKTPYTFDVSVWEFFLPLIAGATLVMARPEGHKDVAYLVRVMEEQRITTCHFVPSMLNFFLKEPALPTHLRQVFTSGEALSYDVMDTFLRRSPARLHNLYGPTEAAVDVTYWPCERRPDRKVPIGRAISNVEIHILDSALRPVPAGAEGDLYIGGVCLARGYLNRPELSRERFVPSPFDPGARLYNTGDRARTLDDGNIEYLGRLDAQVKLRGFRIELGEIEAALSAHEAVQDAVVAVQDAHTEDPKLVAYLVTGGRPFPAPGALKAYLKERLPDYMVPNRFAPIAQIPVTAHGKLDRKALPWPVPAPSAQPEPPPAGAAAAPPGAAQARRPAGVSREAAEEELQRILGKALHLTRLDPGADLFELGATSLTIVQASQHIEERFGVGLPVEVVLAEPTLDAIARHVAERTAAGAPEPPAPGPALDAPPAAPEPPAAAAPGPIDFFSREDRERFKQQQLHLRHGVEGLPTVDLADAPAAPRLYRDRGSRRDYRPEPVSFDDLSRLLAVLRRYPSGQQTQLCYPSAGGTYAVQTYLHVKEGAVERLPAGIYYYHPDRNQLVLINDRPAIRRVHHFYYNREHFDRAGFGLFFIAQTDAIQPIYGDQSLTFAAIEAGAMIQALMSHQAEADLGLCAMGGLDFDAIRADFKLGSGHRYIVCMLGGRVDREGGGRQGRARLLESAGADGSYGAAAAEAAAPRREREAPAGAREIAVIGLAGRYPGADTPRQLWRALRSGQSAVTRPPAGRFGASAPQGDEPRGGGASPGWGGYLERLDRFDSLFFGISPAEAKLMDPQERLFIEVAWECLEDAGYTPEELRRAAPRVGVFVGAMWSDYQSVGLEAWQRDRRAKAVAFHSSIANRISYLFDLHGPSVAIDTSCSSGLTALHLASRSLRLGECDVALVGGVNLLGHPFHPDLLEGLNLTSRDDKTRAFGAGGSGWVPGEGVGAVLLRRLPEAEERGEHIRCVLKGTALAHAGKAPRYGMPSTRAQAGSIRDALADGGVAASEIDYVECAATGSGIADASEVDALKQAFEGRSPDGPPCLLGSVKPNIGHLESASALSQLTKVILQLEHGEIAPTLHTEPRNPLIQLDGTPFRINRALSPWPRAAGADAPPRRALINAFGATGSSAHAVVEEYRPRRRASTPAAAVPGLFVFVLSADTAEQLEACARALAEHLRERSTARPRDVAPPAAAADVAYTLQVGRRAMDERLAILAGDLDELEARLRGFLGGRGEDDGEHLFRGRASSPRDRAPLSPEAPLPALARAWVNGASIAWHDLYTDGSRRRVPLPTYPFARPSHWLGRPAGDAAAPAVARGETAEEAPSRGETAEEAPSRGETAEEAPSRETAEEAPAALAPATADPALRKATLGLLSSCFAEVAEIPRRSLDPEVPLDRYGLNSMLIAQLSARLEALLGELPKTLLFEHHTLAALTDWLVAHRGDALLRRLDLPRRAAGPAASPGALPAAPAARRGPARERSAAASPALAPAAPLESVDIAIVGLSGRYPGADTIDAFWSNLRQGRDSVTEVPADRWDAAAIFDPEGGPGKTRQRWGGFLDRVDRFDALLFNISPREAAGMDPQERLFLEIAWCAFEDAVYTRERLAEEQARAGVGAGVFVGSMYQQYSMLARTPDAGASSSFWSIANRVSYFFDLRGPSLAVDTACASSLTALHLACESLRRGECCLALAGGVNLHLHPHKYVALDRLGLLGSGAASKSLGDGDGYVPGEAVGAVVLKPLDRAVADNDRIYGVIKGSFANHAGKTAGYGVPSPAAQADLIAAALRRTGIDPETIGYIEVAANGSSLGDAIELAGLTQAFRRFTARKHFCAVGSVKSNIGHPEAASGIAQLTKVLGQLHHRTLVPTLHAEPHNPNIDLRDSPFYVQRELGPWTAPTLAGEGGTAELPRRAAISSFGAGGANTHLLVEEYSPRPDDRGDEGAVPGAVIVPLSARTAGQLRAYAATLADDLERRSRPRGHGERALADRDLTAVAYTLQVGREAMNERSAIVTASLGDLITKLRQLAAGQTDVDDLHVGSAAASLSALMLDGREGQAFLSILVEDGRHDKLARLWVSGARIDWRTLYGGSTPRPLSLPHYPFAGDRHWLDDEALPHGAAWSATAAPPAQTAAWSATAAPPARAADPGGAAPPEGPGGAPPGGAARQRIAQELTAMVCDVLKMQARDVDGDEALRNYGMDSRLSAAFMRSVQQRYGSSVPLSAAHTHPTLNQLTAHIHGLLSSNGAARHPSAAPLAATSPSIATAPAASAAPAASAAPAASAAPAASAAPAASAAPAASAASAVPAALHEAPAPDPRAGDARPGADSIAPQPEPGPNPDELVVINPRGSRGSSFWVHGAPGLAQPLYPLSAALGTDYPFFAFQARGVDGLAMPFTSIEEIAAHYVACLRQRSPRGPYVVGGLSSGGIIAFEMARQLLSQGERVSRLVMLDTYPAVAGLAQETPGDIDPILPLLLMANSFISFDRDGDTAIKPDDLAGLPPPMQLPRAVQLIKERSRTALSRDQIYRMLNGNIAVYKHLDLACRKYQPGVLDAVDVLFFKAEKGFFGGANPLGLPILDVFSSYDYVTPWRQWIRGGLQVVELPCAHVDLLEPPALHQVVAHVREALS

SEQ ID NO: 11 - protein, 273 aa, Sorangium cellulosum
MTSMARHLDIHEELPQTAPLPPRAIQWRKAFRLAKELTEKPFTAELSYELILSLDGGATERMFQDFLAEPGARALIQKRPDLAATLSDLDLLGSMPEGSLGRTYKEMTERDGYAVNGIIHVMKAVPTFQEVAPDPLRQWFSFRGAVLHDVAHALTGYGRDLAGEVALGLYLAAVYPPYRSGVVYSFITALASVTAPQDQKLRNLSYLRDVWIRGRRSRIPLSAPWEDLLPLQVEEVCRMYQVPLVRETHAEGILRDAFEKGPWIPSFKAQSWA

SEQ ID NO: 12 - protein, 841 aa, Sorangium cellulosum
MKAYMFPGQGSQAKGMGRALFDAFPALTARADGVLGYSIRALCQDDPDQRLSQTQFTQPALYVVNALSYLKRREEEAPPDFLAGHSLGEFSALFAAGVFDFETGLALVKKRGELMGDARGGGMAAVIGLDEERVRELLDQNGATAVDIANLNSPSQVVISGAKDEIARLQVPFEAAGAKKYTVLRVSAAFHSRFMRPAMVEFGRFLEGYDFAPPKIPVISNVTARPCKADGIRAALSEQIASPVRWCESIRYLMGRGVEEFVECGHGIVLTGLYAQIRRDAQPLVVDEGAAGLDRRGPPAEGRSPAAFGSSRLAAPAQNGAAAPAQNGAAAPAPAAHAAAAHAPAQNGAAAPAQNGAAAPAPAARAAAAHAAAPNGAASPEPAAPAPRGARRISLEVLGSAAFREDYRLRYAYVAGSLVDGISSKEMIVRMGKAGLIGYLGTKGVALDAVEASILHIQRELRGGESYGVSLWCDMDDSHLEWQSVALYLKHDIRYVEAVAYMQITPALVCYRLKGAHRDHRGRAATPRRVLARVSNLEVARAFMSPAADHVLDQLVKDGRLTREEGALGRELPISDDLCAHADSGGPTDMGTAAVLMPAMARLRDDMMTRYGYEKRIRVGMAGGLGAPEAVASAFMLGADFIVTNSVNQCSPEASTSDRVKDMLQAASVHDTAYAPAGDLFEMGARVQVLKRGVLFPARANRLYELYRHYPSLDALDARTRDQLEKHYFRRDLDDVWRDALSRRPGTRPADAARTERDPKHRMSLVFRWYFAHCSELARRGDEENRVNYQVHCGPAMGAFNQWAKGTDLEDWRNRHVDVIAERLMRASADLLDHRMRALSR